(19) United States
(12) Patent Application Publication
(10) Pub. No.: US 2026/0297086 A1
(43) Pub. Date: Oct. 1, 2026

(54) CRYSTALLINE PROLYL HYDROXYLASE DOMAIN-CONTAINING PROTEIN (PHD) INHIBITOR AND USES THEREOF

(71) Applicant: Insilico Medicine IP Limited, Hong Kong (CN)

(72) Inventors: Yushu YIN, Shanghai (CN); Jianyu XU, Shanghai (CN); Xing LIANG, Shanghai (CN); Xiao DING, Shanghai (CN)

(21) Appl. No.: 19/477,735

(22) PCT Filed: Apr. 26, 2024
(86) PCT No.: PCT/CN2024/090097

§ 371 Date: Oct. 22, 2025

(30) Foreign Application Priority Data
Apr. 28, 2023 (WO) PCT/CN2023/091755

(51) Int. Cl. C07D 471/04; A61K 31/496

(52) U.S. Cl. CPC C07D 471/04; A61K 31/496

(57) ABSTRACT

Described herein are crystalline forms Compound (I), of a small molecule prolyl hydroxylase domain-containing protein (PHD) inhibitor, as well as pharmaceutical compositions thereof, and methods of use thereof in the treatment of diseases or conditions that would benefit from treatment with a prolyl hydroxylase domain-containing protein (PHD) inhibitor.

### CROSS-REFERENCE

This patent application claims the benefit of International Application No. PCT/CN2023/091755, filed Apr. 28, 2023; which is incorporated herein by reference in its entirety.

### BACKGROUND

Hypoxia-inducible factor (HIF) mediates gene expression in response to changes in cellular oxygen concentration. HIF is a heterodimer having an oxygen-regulated subunit (HIF-α) and a constitutively expressed subunit (HIF-β). HIF prolyl hydroxylase, which is also known as prolyl hydroxylase domain-containing protein (PHD), exists as three isoforms in humans (PHD1, PHD2, and PHD3). PHDs act as oxygen sensors modulating the hypoxia-inducible factor (“HIF”) degradation pathway. Briefly, PHDs are responsible for hydroxylation of HIFα, a subunit of HIF, which initiates the pathway that eventually results in the degradation of HIFα by the proteasome. There are three subtypes of PHDs, including PHD1, PHD2 and PHD3. Inhibition of PHDs has been indicated as a promising therapy for the HIFα related disease, such as inflammatory bowel disease (IBD).

Inhibitors of PHDs coordinate erythropoiesis by inducing both renal and hepatic erythropoietin (“EPO”) synthesis, which stimulates the production of red blood cells in the bone marrow, and by regulating the metabolism of iron, an indispensable component of functional red blood cells. Inhibitors of PHDs could also suppress the production of hepatic hepcidin, which has negative effects on iron mobilization. It is also speculated that inhibitors of PHDs might upregulate the expression several iron metabolism gene, such as DMT1 and DCYTB. Because of the central role HIF prolyl hydrolase plays in cellular oxygen sensing, inhibitors of PHD may be useful in treating cardiovascular disorders, metabolic disorders, hematological disorders, pulmonary disorders, kidney disorders, liver disorders, wound healing disorders, and cancer, among others.

### SUMMARY

Disclosed herein is a solid state form of tert-butyl 4-(6-(((6-cyanopyridin-3-yl)methyl)carbamoyl)-5-hydroxy-1,7-naphthyridin-2-yl)piperazine-1-carboxylate:

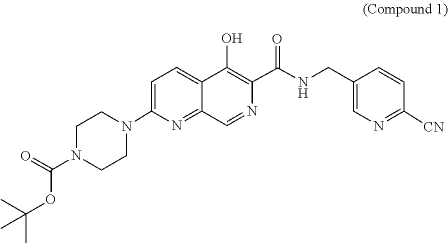 [SMILES: CC(C)(C)OC(=O)N1CCN(c2ccc3c(O)c(C(=O)NCc4ccc(C#N)nc4)ncc3n2)CC1]

or a pharmaceutically acceptable salt thereof.

In some embodiments, the solid state form is a crystalline form.

In some embodiments, the solid state form is crystalline Compound 1 as a freebase.

In some embodiments, the solid state form is crystalline Compound 1 freebase Type C.

In some embodiments, the solid state form is crystalline Compound 1 freebase Type A, crystalline Compound 1 freebase Type B, crystalline Compound 1 freebase Type C, crystalline Compound 1 freebase Type D, crystalline Compound 1 freebase Type E, crystalline Compound 1 freebase Type F, or crystalline Compound 1 freebase Type 1.

Also disclosed herein is a pharmaceutical composition comprising a therapeutically effective amount of a crystalline form disclosed herein and a pharmaceutically acceptable excipient.

Also disclosed herein is a method of treating a disease or disorder in a subject, the method comprising administering to the subject a crystalline form disclosed herein or a pharmaceutical composition disclosed herein, wherein the disease or disorder is inflammatory bowel disease (IBD). In some embodiments, the disease or disorder is ulcerative colitis (“UC”) or Crohn's disease (“CD”).

### BRIEF DESCRIPTION OF THE DRAWINGS

The features of the invention are set forth with particularity in the appended claims. A better understanding of the features of the present invention will be obtained by reference to the following detailed description that sets forth illustrative embodiments, in which the principles of the invention are utilized, and the accompanying drawings of which:

FIG. 1 shows the X-Ray Powder Diffraction (XRPD) pattern of Compound 1 freebase Type A.

FIG. 2 shows the Differential Scanning Calorimetry (DSC) thermogram of Compound 1 freebase Type A.

FIG. 3 shows the Thermogravimetric Analysis (TGA) thermogram of Compound 1 freebase Type A.

FIG. 4 shows the X-Ray Powder Diffraction (XRPD) pattern of Compound 1 freebase Type 1.

FIG. 5 shows the Differential Scanning Calorimetry (DSC) thermogram of Compound 1 freebase Type 1.

FIG. 6 shows the Thermogravimetric Analysis (TGA) thermogram of Compound 1 freebase Type 1.

FIG. 7 shows the X-Ray Powder Diffraction (XRPD) pattern of Compound 1 freebase Type B.

FIG. 8 shows the Differential Scanning Calorimetry (DSC) thermogram of Compound 1 freebase Type B.

FIG. 9 shows the Thermogravimetric Analysis (TGA) thermogram of Compound 1 freebase Type B.

FIG. 10 shows the X-Ray Powder Diffraction (XRPD) pattern of Compound 1 freebase Type C.

FIG. 11 shows the Differential Scanning Calorimetry (DSC) thermogram of Compound 1 freebase Type C.

FIG. 12 shows the Thermogravimetric Analysis (TGA) thermogram of Compound 1 freebase Type C.

FIG. 13 shows the X-Ray Powder Diffraction (XRPD) pattern of Compound 1 freebase Type D.

FIG. 14 shows the Differential Scanning Calorimetry (DSC) thermogram of Compound 1 freebase Type D with a heating rate of 2 [symbol] /min.

FIG. 152 shows the Thermogravimetric Analysis (TGA) thermogram of Compound 1 freebase Type D.

FIG. 16 shows the X-Ray Powder Diffraction (XRPD) pattern of Compound 1 freebase Type E.

FIG. 173 shows the Differential Scanning Calorimetry (DSC) thermogram of Compound 1 freebase Type E.

FIG. 184 shows the Thermogravimetric Analysis (TGA) thermogram of Compound 1 freebase Type E.

FIG. 19 shows the X-Ray Powder Diffraction (XRPD) pattern of Compound 1 freebase Type F.

FIG. 20 shows the Differential Scanning Calorimetry (DSC) thermogram of Compound 1 freebase Type F.

FIG. 21 shows the Thermogravimetric Analysis (TGA) thermogram of Compound 1 freebase Type F.

FIG. 22 shows the X-Ray Powder Diffraction (XRPD) pattern of Compound 1 HCl salt Pattern A.

FIG. 23 shows the Differential Scanning Calorimetry (DSC) thermogram of Compound 1 HCl salt Pattern A.

FIG. 24 shows the Thermogravimetric Analysis (TGA) thermogram of Compound 1 HCl salt Pattern A.

FIG. 25 shows the X-Ray Powder Diffraction (XRPD) pattern of Compound 1 Sulfate salt Pattern A.

FIG. 26 shows the Differential Scanning Calorimetry (DSC) thermogram of Compound 1 Sulfate salt Pattern A.

FIG. 27 shows the Thermogravimetric Analysis (TGA) thermogram of Compound 1 Sulfate salt Pattern A.

FIG. 28 shows the X-Ray Powder Diffraction (XRPD) pattern of Compound 1 Sulfate salt Pattern B.

FIG. 29 shows the Differential Scanning Calorimetry (DSC) thermogram of Compound 1 Sulfate salt Pattern B.

FIG. 30 shows the Thermogravimetric Analysis (TGA) thermogram of Compound 1 Sulfate salt Pattern B.

FIG. 31 shows the X-Ray Powder Diffraction (XRPD) pattern of Compound 1 methanesulfonate salt Pattern A.

FIG. 32 shows the Differential Scanning Calorimetry (DSC) thermogram of Compound 1 methanesulfonate salt Pattern A.

FIG. 33 shows the Thermogravimetric Analysis (TGA) thermogram of Compound 1 methanesulfonate salt Pattern A.

FIG. 34 shows the X-Ray Powder Diffraction (XRPD) pattern of Compound 1 benzenesulfonate salt Pattern A.

FIG. 35 shows the Differential Scanning Calorimetry (DSC) thermogram of Compound 1 benzenesulfonate salt Pattern A.

FIG. 36 shows the Thermogravimetric Analysis (TGA) thermogram of Compound 1 benzenesulfonate salt Pattern A.

FIG. 37 shows the X-Ray Powder Diffraction (XRPD) pattern of Compound 1 naphtalene-1,5-disulfonate salt Pattern A.

FIG. 38 shows the Differential Scanning Calorimetry (DSC) thermogram of Compound 1 naphtalene-1,5-disulfonate salt Pattern A.

FIG. 39 shows the Thermogravimetric Analysis (TGA) thermogram of Compound 1 naphtalene-1,5-disulfonate salt Pattern A.

FIG. 40 shows the X-Ray Powder Diffraction (XRPD) pattern of Compound 1 naphtalene-1,5-disulfonate salt Pattern B.

FIG. 41 shows the Differential Scanning Calorimetry (DSC) thermogram of Compound 1 naphtalene-1,5-disulfonate salt Pattern B.

FIG. 42 shows the Thermogravimetric Analysis (TGA) thermogram of Compound 1 naphtalene-1,5-disulfonate salt Pattern B.

### DETAILED DESCRIPTION

While small molecule inhibitors are often initially evaluated for their activity when dissolved in solution, solid state characteristics such as polymorphism are also important. Polymorphic forms of a drug substance can have different physical properties, including melting point, apparent solubility, dissolution rate, optical and mechanical properties, vapor pressure, and density. These properties can have a direct effect on the ability to process or manufacture a drug substance and the drug product. Moreover, differences in these properties can and often lead to different pharmacokinetics profiles for different polymorphic forms of a drug. Therefore, polymorphism is often an important factor under regulatory review of the ‘sameness’ of drug products from various manufacturers.

### Compound 1

Compound 1 is tert-butyl 4-(6-(((6-cyanopyridin-3-yl)methyl)carbamoyl)-5-hydroxy-1,7-naphthyridin-2-yl)piperazine-1-carboxylate:

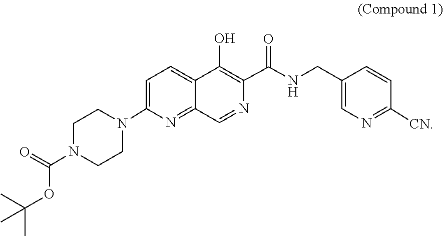 [SMILES: CC(C)(C)OC(=O)N1CCN(c2ccc3c(O)c(C(=O)NCc4ccc(C#N)nc4)ncc3n2)CC1]

In some embodiments, Compound 1 is in the form of a freebase. In some embodiments, Compound 1 is in the form of a pharmaceutically acceptable salt. In some embodiments, Compound 1 is in the form of an HCl salt. In some embodiments, Compound 1 is in the form of an sulfate salt. In some embodiments, Compound 1 is in the form of an methanesulfonate salt. In some embodiments, Compound 1 is in the form of an benzenesulfonate salt. In some embodiments, Compound 1 is in the form of an naphthalene-1,5-disulfonate salt.

### Solid State Form of Compound 1

In one aspect, provided herein is a solid state form of tert-butyl 4-(6-(((6-cyanopyridin-3-yl)methyl)carbamoyl)-5-hydroxy-1,7-naphthyridin-2-yl)piperazine-1-carboxylate:

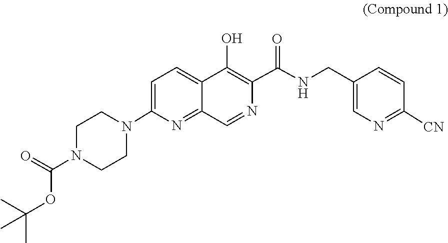 [SMILES: CC(C)(C)OC(=O)N1CCN(c2ccc3c(O)c(C(=O)NCc4ccc(C#N)nc4)ncc3n2)CC1]

or a pharmaceutically acceptable salt thereof.

In some embodiments, the solid state form is a crystalline form.

In some embodiments, the solid state form is crystalline Compound 1 as a freebase. In some embodiments, the solid state form is crystalline Compound 1 freebase Type A. In some embodiments, the solid state form is crystalline Compound 1 freebase Type B. In some embodiments, the solid state form is crystalline Compound 1 freebase Type C. In some embodiments, the solid state form is crystalline Compound 1 freebase Type D. In some embodiments, the solid state form is crystalline Compound 1 freebase Type E. In some embodiments, the solid state form is crystalline Compound 1 freebase Type F. In some embodiments, the solid state form is crystalline Compound 1 freebase Type 1.

In some embodiments, the solid state form is Compound 1 HCl salt. In some embodiments, the solid state form is crystalline Compound 1 HCl salt. In some embodiments, the solid state form is crystalline Compound 1 HCl salt Type A.

In some embodiments, the solid state form is Compound 1 sulfate salt. In some embodiments, the solid state form is crystalline Compound 1 sulfate salt. In some embodiments, the solid state form is crystalline Compound 1 sulfate salt Type A. In some embodiments, the solid state form is crystalline Compound 1 sulfate salt Type B.

In some embodiments, the solid state form is Compound 1 methanesulfonate salt. In some embodiments, the solid state form is crystalline Compound 1 methanesulfonate salt. In some embodiments, the solid state form is crystalline Compound 1 methanesulfonate salt Type A.

In some embodiments, the solid state form is Compound 1 benzenesulfonate salt. In some embodiments, the solid state form is crystalline Compound 1 benzenesulfonate salt. In some embodiments, the solid state form is crystalline Compound 1 benzenesulfonate salt Type A.

In some embodiments, the solid state form is Compound 1 naphthalene-1,5-disulfonate salt. In some embodiments, the solid state form is crystalline Compound 1 naphthalene-1,5-disulfonate salt. In some embodiments, the solid state form is crystalline Compound 1 naphthalene-1,5-disulfonate salt Type A. In some embodiments, the solid state form is crystalline Compound 1 naphthalene-1,5-disulfonate salt Type B.

### Compound 1 Freebase Type A

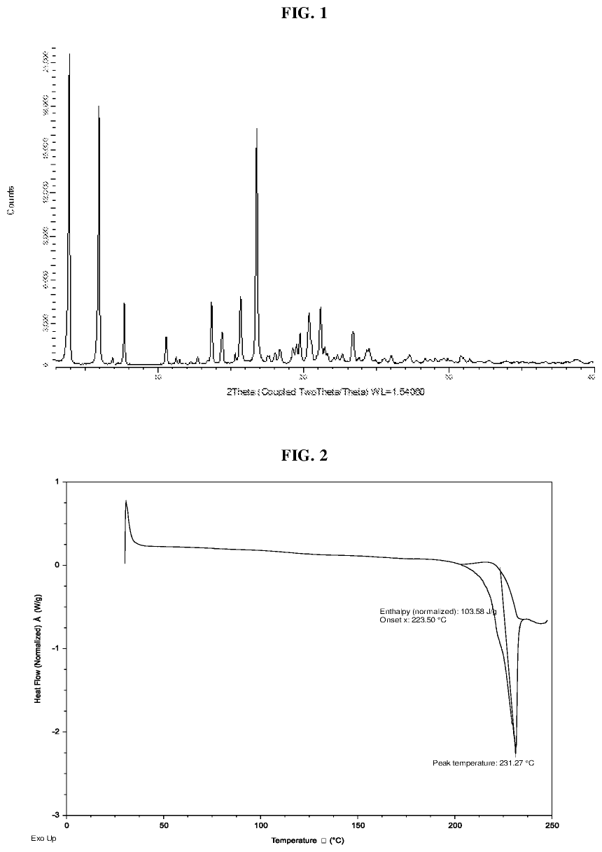

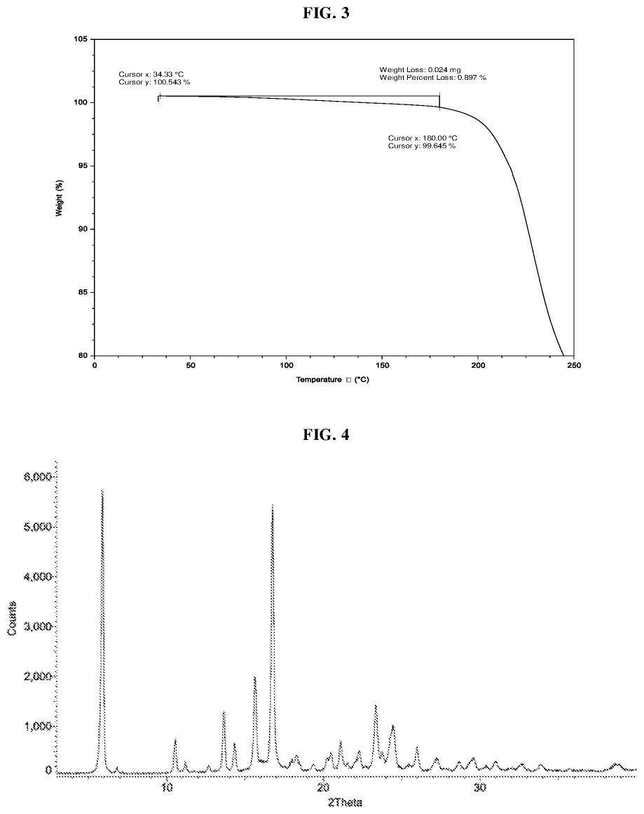

Disclosed herein is Compound 1 freebase Type A. In some embodiments, the crystalline form is Compound 1 freebase characterized as having at least one of the following properties:
(a) an X-Ray powder diffraction (XRPD) pattern substantially the same as shown in FIG. 1 as measured using Cu Kα.radiation;
(b) an X-Ray powder diffraction (XRPD) pattern with peaks at 3.8±0.2° 2θ, 5.9±0.2° 2θ, and 16.7±0.2° 2θ as measured using Cu Kα.radiation;
(c) a Differential Scanning Calorimetry (DSC) thermogram substantially the same as shown in FIG. 2;
(d) a Differential Scanning Calorimetry (DSC) thermogram with an endothermic peak having a peak temperature at about 223.5 [symbol] ;
(e) a Thermogravimetric Thermal Analysis (TGA) thermogram substantially the same as shown in FIG. 3; or
(f) combinations thereof.

In some embodiments, the crystalline form is Compound 1 freebase characterized as having at least one of the following properties:
(a) an X-Ray powder diffraction (XRPD) pattern substantially the same as shown in FIG. 1 as measured using Cu Kα.radiation;
(b) an X-Ray powder diffraction (XRPD) pattern with peaks at 3.8±0.2° 2θ, 5.9±0.2° 2θ, and 16.7±0.2° 2θ as measured using Cu Kα.radiation;
(c) a Differential Scanning Calorimetry (DSC) thermogram substantially the same as shown in FIG. 2;
(d) a Thermogravimetric Thermal Analysis (TGA) thermogram substantially the same as shown in FIG. 3; or
(e) combinations thereof.

In some embodiments, the crystalline form is Compound 1 freebase characterized as having at least one of the following properties:
(a) an X-Ray powder diffraction (XRPD) pattern with peaks at 3.8±0.2° 2θ, 5.9±0.2° 2θ, and 16.7±0.2° 2θ as measured using Cu Kα.radiation;
(b) a Differential Scanning Calorimetry (DSC) thermogram with an endothermic peak having a peak temperature at about 223.5 [symbol] ; or
(c) combinations thereof.

In some embodiments of Compound 1 freebase, the crystalline form has an X-ray powder diffraction (XRPD) pattern substantially the same as shown in FIG. 1 as measured using Cu Kα.radiation.

In some embodiments of Compound 1 freebase, the crystalline form has an X-ray powder diffraction (XRPD) pattern with peaks found in Table 1 as measured using Cu Kα.radiation.

In some embodiments of Compound 1 freebase, the crystalline form has an X-ray powder diffraction (XRPD) pattern with peaks at 3.8±0.2° 2θ, 5.9±0.2° 2θ, and 16.7±0.2° 2θ as measured using Cu Kα.radiation.

In some embodiments of Compound 1 freebase, the X-ray powder diffraction (XRPD) pattern further comprises peaks at 7.6±0.2° 2θ, 10.5±0.2° 2θ, 13.6±0.2° 2θ, and 15.6±0.2° 2θ as measured using Cu Kα.radiation.

In some embodiments of Compound 1 freebase, the crystalline form has an X-ray powder diffraction (XRPD) pattern with peaks at 3.8±0.2° 2θ, 5.9±0.2° 2θ, 7.6±0.2° 2θ, 10.5±0.2° 2θ, 13.6±0.2° 2θ, 15.6±0.2° 2θ, and 16.7±0.2° 2θ as measured using Cu Kα.radiation.

In some embodiments of Compound 1 freebase, the crystalline form has an X-ray powder diffraction (XRPD) pattern with at least two peaks selected from 3.8±0.2° 2θ, 5.9±0.2° 2θ, 7.6±0.2° 2θ, 10.5±0.2° 2θ, 13.6±0.2° 2θ, 15.6±0.2° 2θ, and 16.7±0.2° 2θ as measured using Cu Kα.radiation.

In some embodiments of Compound 1 freebase, the crystalline form has an X-ray powder diffraction (XRPD) pattern with at least three peaks selected from 3.8±0.2° 2θ, 5.9±0.2° 2θ, 7.6±0.2° 2θ, 10.5±0.2° 2θ, 13.6±0.2° 2θ, 15.6±0.2° 2θ, and 16.7±0.2° 2θ as measured using Cu Kα.radiation.

In some embodiments of Compound 1 freebase, the crystalline form has an X-ray powder diffraction (XRPD) pattern with at least four peaks selected from 3.8±0.2° 2θ, 5.9±0.2° 2θ, 7.6±0.2° 2θ, 10.5±0.2° 2θ, 13.6±0.2° 2θ, 15.6±0.2° 2θ, and 16.7±0.2° 2θ as measured using Cu Kα.radiation.

In some embodiments of Compound 1 freebase, the crystalline form has an X-ray powder diffraction (XRPD) pattern with at least five peaks selected from 3.8±0.2° 2θ, 5.9±0.2° 2θ, 7.6±0.2° 2θ, 10.5±0.2° 2θ, 13.6±0.2° 2θ, 15.6±0.2° 2θ, and 16.7±0.2° 2θ as measured using Cu Kα.radiation.

In some embodiments of Compound 1 freebase, the crystalline form has an X-ray powder diffraction (XRPD) pattern with at least six peaks selected from 3.8±0.2° 2θ, 5.9±0.2° 2θ, 7.6±0.2° 2θ, 10.5±0.2° 2θ, 13.6±0.2° 2θ, 15.6±0.2° 2θ, and 16.7±0.2° 2θ as measured using Cu Kα.radiation.

In some embodiments of Compound 1 freebase, the Differential Scanning Calorimetry (DSC) thermogram is substantially the same as shown in FIG. 2.

In some embodiments of Compound 1 freebase, the Differential Scanning Calorimetry (DSC) thermogram has an endothermic peak having a peak temperature at about 223.5 [symbol] .

In some embodiments of Compound 1 freebase, the Thermogravimetric Thermal Analysis (TGA) thermogram is substantially the same as shown in FIG. 3.

TABLE 1
XRPD peaks table of Compound 1 FreebasType A

| Peak# | Diffraction angle (°, 2 θ) | Rel. Int. % |
|---|---|---|
| 1 | 3.8 | 38.00% |
| 2 | 5.9 | 72.10% |
| 3 | 6.8 | 1.60% |
| 4 | 7.6 | 13.20% |
| 5 | 10.5 | 10.30% |
| 6 | 11.2 | 2.50% |
| 7 | 11.4 | 1.80% |
| 8 | 12.2 | 0.70% |
| 9 | 12.7 | 2.10% |
| 10 | 13.6 | 24.50% |
| 11 | 14.3 | 11.60% |
| 12 | 15.2 | 3.30% |
| 13 | 15.6 | 30.40% |
| 14 | 16.0 | 2.30% |
| 15 | 16.7 | 100.00% |
| 16 | 17.1 | 3.10% |
| 17 | 17.5 | 2.90% |
| 18 | 18.0 | 4.60% |
| 19 | 18.3 | 6.30% |
| 20 | 19.3 | 4.00% |
| 21 | 19.4 | 7.30% |
| 22 | 19.7 | 7.80% |
| 23 | 20.3 | 13.30% |
| 24 | 21.1 | 17.80% |
| 25 | 21.6 | 3.10% |
| 26 | 22.0 | 1.60% |
| 27 | 22.2 | 4.30% |
| 28 | 22.6 | 2.80% |
| 29 | 23.3 | 18.10% |
| 30 | 23.7 | 2.50% |
| 31 | 24.3 | 8.70% |
| 32 | 24.5 | 10.80% |
| 33 | 25.5 | 2.30% |
| 34 | 26.0 | 6.20% |
| 35 | 27.2 | 3.70% |
| 36 | 27.7 | 1.20% |
| 37 | 28.6 | 2.40% |
| 38 | 29.4 | 2.30% |
| 39 | 29.6 | 2.80% |
| 40 | 30.1 | 0.90% |
| 41 | 30.8 | 2.30% |
| 42 | 31.4 | 1.60% |
| 43 | 32.7 | 1.90% |
| 44 | 33.9 | 1.70% |
| 45 | 38.7 | 2.50% |
| 46 | 39.7 | 1.20% |

### Compound 1 Freebase Type 1

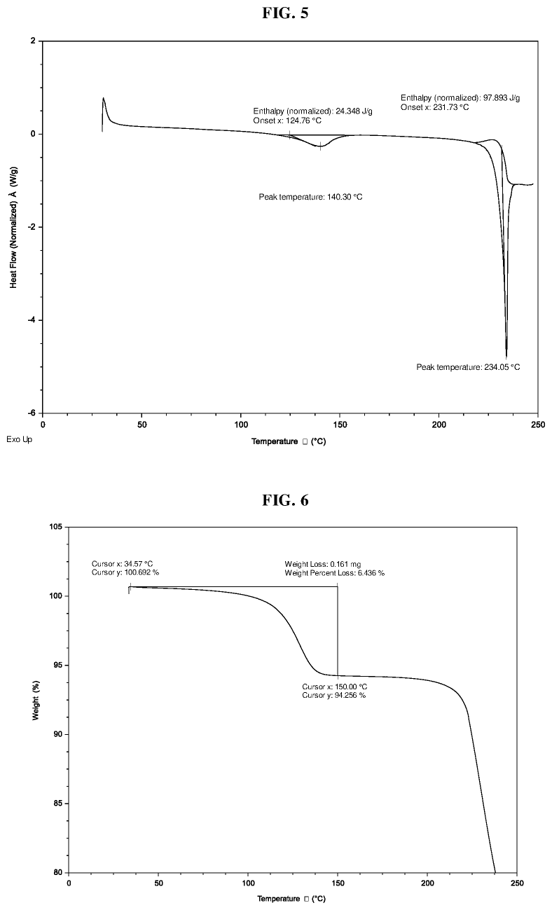

Disclosed herein is Compound 1 freebase Type 1. In some embodiments, the crystalline form is Compound 1 freebase characterized as having at least one of the following properties:
(a) an X-Ray powder diffraction (XRPD) pattern substantially the same as shown in FIG. 4 as measured using Cu Kα.radiation;
(b) an X-Ray powder diffraction (XRPD) pattern with peaks at 5.9±0.2° 2θ, 15.6±0.2° 2θ, and 16.8±0.2° 2θ as measured using Cu Kα.radiation;
(c) a Differential Scanning Calorimetry (DSC) thermogram substantially the same as shown in FIG. 5;
(d) a Differential Scanning Calorimetry (DSC) thermogram with an endothermic peak having a peak temperature at about 124.8 [symbol] ;
(e) a Thermogravimetric Thermal Analysis (TGA) thermogram substantially the same as shown in FIG. 6; or
(f) combinations thereof.

In some embodiments, the crystalline form is Compound 1 freebase characterized as having at least one of the following properties:
(a) an X-Ray powder diffraction (XRPD) pattern substantially the same as shown in FIG. 4 as measured using Cu Kα.radiation;
(b) an X-Ray powder diffraction (XRPD) pattern with peaks at 5.9±0.2° 2θ, 15.6±0.2° 2θ, and 16.8±0.2° 2θ as measured using Cu Kα.radiation;
(c) a Differential Scanning Calorimetry (DSC) thermogram substantially the same as shown in FIG. 5;
(d) a Thermogravimetric Thermal Analysis (TGA) thermogram substantially the same as shown in FIG. 6; or
(e) combinations thereof.

In some embodiments, the crystalline form is Compound 1 freebase characterized as having at least one of the following properties:
(a) an X-Ray powder diffraction (XRPD) pattern with peaks at 5.9±0.2° 2θ, 15.6±0.2° 2θ, and 16.8±0.2° 2θ as measured using Cu Kα.radiation;
(b) a Differential Scanning Calorimetry (DSC) thermogram with an endothermic peak having a peak temperature at about 124.8 [symbol] ;
(c) combinations thereof.

In some embodiments of Compound 1 freebase, the crystalline form has an X-ray powder diffraction (XRPD) pattern substantially the same as shown in FIG. 4 as measured using Cu Kα.radiation.

In some embodiments of Compound 1 freebase, the crystalline form has an X-ray powder diffraction (XRPD) pattern with peaks found in Table 2 as measured using Cu Kα.radiation.

In some embodiments of Compound 1 freebase, the crystalline form has an X-ray powder diffraction (XRPD) pattern with peaks at 5.9±0.2° 2θ, 15.6±0.2° 2θ, and 16.8±0.2° 2θ as measured using Cu Kα.radiation.

In some embodiments of Compound 1 freebase, the X-ray powder diffraction (XRPD) pattern further comprises peaks at 13.6±0.2° 2θ, 14.3±0.2° 2θ, and 23.4±0.2° 2θ as measured using Cu Kα.radiation.

In some embodiments of Compound 1 freebase, the crystalline form has an X-ray powder diffraction (XRPD) pattern with peaks at 5.9±0.2° 2θ, 13.6±0.2° 2θ, 14.3±0.2° 2θ, 15.6±0.2° 2θ, 16.8±0.2° 2θ, and 23.4±0.2° 2θ as measured using Cu Kα.radiation.

In some embodiments of Compound 1 freebase, the crystalline form has an X-ray powder diffraction (XRPD) pattern with at least two peaks selected from 5.9±0.2° 2θ, 13.6±0.2° 2θ, 14.3±0.2° 2θ, 15.6±0.2° 2θ, 16.8±0.2° 2θ, and 23.4±0.2° 2θ as measured using Cu Kα.radiation.

In some embodiments of Compound 1 freebase, the crystalline form has an X-ray powder diffraction (XRPD) pattern with at least three peaks selected from 5.9±0.2° 2θ, 13.6±0.2° 2θ, 14.3±0.2° 2θ, 15.6±0.2° 2θ, 16.8±0.2° 2θ, and 23.4±0.2° 2θ as measured using Cu Kα.radiation.

In some embodiments of Compound 1 freebase, the crystalline form has an X-ray powder diffraction (XRPD) pattern with at least four peaks selected from 5.9±0.2° 2θ, 13.6±0.2° 2θ, 14.3±0.2° 2θ, 15.6±0.2° 2θ, 16.8±0.2° 2θ, and 23.4±0.2° 2θ as measured using Cu Kα.radiation.

In some embodiments of Compound 1 freebase, the crystalline form has an X-ray powder diffraction (XRPD) pattern with at least five peaks selected from 5.9±0.2° 2θ, 13.6±0.2° 2θ, 14.3±0.2° 2θ, 15.6±0.2° 2θ, 16.8±0.2° 2θ, and 23.4±0.2° 2θ as measured using Cu Kα.radiation.

In some embodiments of Compound 1 freebase, the Differential Scanning Calorimetry (DSC) thermogram is substantially the same as shown in FIG. 5.

In some embodiments of Compound 1 freebase, the Differential Scanning Calorimetry (DSC) thermogram has an endothermic peak having a peak temperature at about 124.8 [symbol] .

In some embodiments of Compound 1 freebase, the Thermogravimetric Thermal Analysis (TGA) thermogram is substantially the same as shown in FIG. 6.

In some embodiments of Compound 1 freebase, the crystalline form is a solvate.

In some embodiments of Compound 1 freebase, the solvate is an acetone solvate, a methylethylketone solvate, a tetrahydrofuran solvate, an acetonitrile solvate, a dimethylformamide solvate, a dimethylacetamide solvate, a 2-methyltetrahydrofuran solvate, or an ethyl acetate solvate.

TABLE 2
XRPD peaks table of Compound 1 Freebase Type 1

| Peak# | Diffraction angle (°, 2 θ) | Rel. Int. (%) |
|---|---|---|
| 1 | 3.2 | 0.30% |
| 2 | 5.9 | 100.00% |
| 3 | 6.8 | 2.00% |
| 4 | 7.2 | 0.50% |
| 5 | 10.5 | 11.40% |
| 6 | 11.2 | 3.90% |
| 7 | 11.8 | 0.40% |
| 8 | 12.7 | 2.10% |
| 9 | 13.6 | 21.00% |
| 10 | 14.3 | 9.70% |
| 11 | 15.6 | 33.10% |
| 12 | 16.8 | 93.40% |
| 13 | 17.6 | 1.10% |
| 14 | 18.0 | 3.70% |
| 15 | 18.3 | 5.30% |
| 16 | 19.4 | 2.10% |
| 17 | 20.3 | 4.20% |
| 18 | 20.5 | 6.10% |
| 19 | 21.1 | 9.80% |
| 20 | 21.5 | 1.90% |
| 21 | 22.0 | 2.40% |
| 22 | 22.7 | 6.10% |
| 23 | 23.4 | 21.80% |
| 24 | 23.7 | 5.70% |
| 25 | 24.4 | 14.10% |
| 26 | 25.5 | 1.70% |
| 27 | 26.0 | 7.80% |
| 28 | 27.2 | 4.20% |
| 29 | 28.7 | 3.10% |
| 30 | 29.5 | 3.70% |
| 31 | 30.4 | 1.50% |
| 32 | 31.0 | 3.10% |
| 33 | 32.0 | 0.80% |
| 34 | 32.7 | 2.10% |
| 35 | 33.9 | 2.10% |
| 36 | 35.7 | 0.80% |
| 37 | 36.6 | 0.60% |
| 38 | 38.6 | 2.20% |
| 39 | 38.9 | 2.20% |

### Compound 1 Freebase Type B

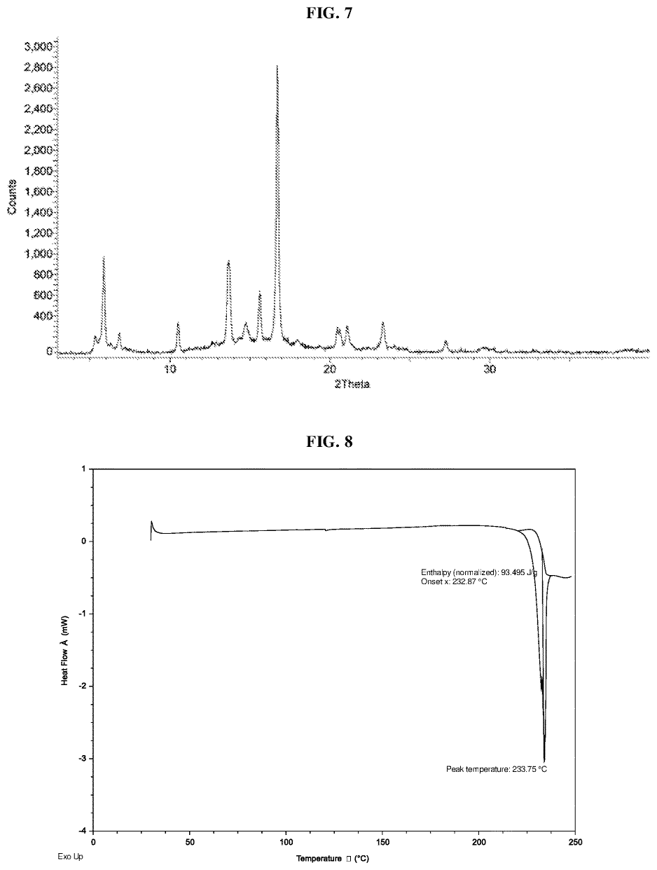

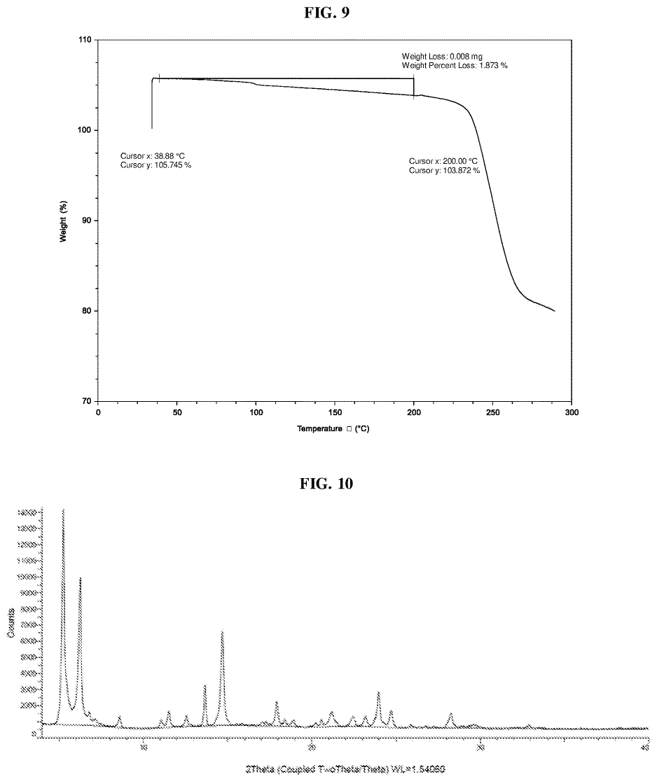

Disclosed herein is Compound 1 freebase Type B. In some embodiments, the crystalline form is Compound 1 freebase characterized as having at least one of the following properties:
(a) an X-Ray powder diffraction (XRPD) pattern substantially the same as shown in FIG. 7 as measured using Cu Kα.radiation;
(b) an X-Ray powder diffraction (XRPD) pattern with peaks at 5.9±0.2° 2θ, 13.7±0.2° 2θ, and 16.7±0.2° 2θ as measured using Cu Kα.radiation;
(c) a Differential Scanning Calorimetry (DSC) thermogram substantially the same as shown in FIG. 8;
(d) a Thermogravimetric Thermal Analysis (TGA) thermogram substantially the same as shown in FIG. 9; or
(e) combinations thereof.

In some embodiments of Compound 1 freebase, the crystalline form has an X-ray powder diffraction (XRPD) pattern substantially the same as shown in FIG. 7 as measured using Cu Kα.radiation.

In some embodiments of Compound 1 freebase, the crystalline form has an X-ray powder diffraction (XRPD) pattern with peaks found in Table 3 as measured using Cu Kα.radiation.

In some embodiments of Compound 1 freebase, the crystalline form has an X-ray powder diffraction (XRPD) pattern with peaks at 5.9±0.2° 2θ, 13.7±0.2° 2θ, and 16.7±0.2° 2θ as measured using Cu Kα.radiation.

In some embodiments of Compound 1 freebase, the X-ray powder diffraction (XRPD) pattern further comprises peaks at 10.5±0.2° 2θ, 15.6±0.2° 2θ, and 23.3±0.2° 2θ as measured using Cu Kα.radiation.

In some embodiments of Compound 1 freebase, the X-ray powder diffraction (XRPD) pattern further comprises peaks at 14.8±0.2° 2θ, 20.5±0.2° 2θ, and 21.1±0.2° 2θ as measured using Cu Kα.radiation.

In some embodiments of Compound 1 freebase, the crystalline form has an X-ray powder diffraction (XRPD) pattern with peaks at 5.9±0.2° 2θ, 10.5±0.2° 2θ, 13.7±0.2° 2θ, 14.8±0.2° 2θ, 15.6±0.2° 2θ, 16.7±0.2° 2θ, 20.5±0.2° 2θ, 21.1±0.2° 2θ, and 23.3±0.2° 2θ, as measured using Cu Kα.radiation.

In some embodiments of Compound 1 freebase, the crystalline form has an X-ray powder diffraction (XRPD) pattern with at least two peaks selected from 5.9±0.2° 2θ, 10.5±0.2° 2θ, 13.7±0.2° 2θ, 14.8±0.2° 2θ, 15.6±0.2° 2θ, 16.7±0.2° 2θ, 20.5±0.2° 2θ, 21.1±0.2° 2θ, and 23.3±0.2° 2θ as measured using Cu Kα.radiation.

In some embodiments of Compound 1 freebase, the crystalline form has an X-ray powder diffraction (XRPD) pattern with at least three peaks selected from 5.9±0.2° 2θ, 10.5±0.2° 2θ, 13.7±0.2° 2θ, 14.8±0.2° 2θ, 15.6±0.2° 2θ, 16.7±0.2° 2θ, 20.5±0.2° 2θ, 21.1±0.2° 2θ, and 23.3±0.2° 2θ as measured using Cu Kα.radiation.

In some embodiments of Compound 1 freebase, the crystalline form has an X-ray powder diffraction (XRPD) pattern with at least four peaks selected from 5.9±0.2° 2θ, 10.5±0.2° 2θ, 13.7±0.2° 2θ, 14.8±0.2° 2θ, 15.6±0.2° 2θ, 16.7±0.2° 2θ, 20.5±0.2° 2θ, 21.1±0.2° 2θ, and 23.3±0.2° 2θ as measured using Cu Kα.radiation.

In some embodiments of Compound 1 freebase, the crystalline form has an X-ray powder diffraction (XRPD) pattern with at least five peaks selected from 5.9±0.2° 2θ, 10.5±0.2° 2θ, 13.7±0.2° 2θ, 14.8±0.2° 2θ, 15.6±0.2° 2θ, 16.7±0.2° 2θ, 20.5±0.2° 2θ, 21.1±0.2° 2θ, and 23.3±0.2° 2θ as measured using Cu Kα.radiation.

In some embodiments of Compound 1 freebase, the crystalline form has an X-ray powder diffraction (XRPD) pattern with at least six peaks selected from 5.9±0.2° 2θ, 10.5±0.2° 2θ, 13.7±0.2° 2θ, 14.8±0.2° 2θ, 15.6±0.2° 2θ, 16.7±0.2° 2θ, 20.5±0.2° 2θ, 21.1±0.2° 2θ, and 23.3±0.2° 2θ as measured using Cu Kα.radiation.

In some embodiments of Compound 1 freebase, the crystalline form has an X-ray powder diffraction (XRPD) pattern with at least seven peaks selected from 5.9±0.2° 2θ, 10.5±0.2° 2θ, 13.7±0.2° 2θ, 14.8±0.2° 2θ, 15.6±0.2° 2θ, 16.7±0.2° 2θ, 20.5±0.2° 2θ, 21.1±0.2° 2θ, and 23.3±0.2° 2θ as measured using Cu Kα.radiation.

In some embodiments of Compound 1 freebase, the crystalline form has an X-ray powder diffraction (XRPD) pattern with at least eight peaks selected from 5.9±0.2° 2θ, 10.5±0.2° 2θ, 13.7±0.2° 2θ, 14.8±0.2° 2θ, 15.6±0.2° 2θ, 16.7±0.2° 2θ, 20.5±0.2° 2θ, 21.1±0.2° 2θ, and 23.3±0.2° 2θ as measured using Cu Kα.radiation.

In some embodiments of Compound 1 freebase, the Differential Scanning Calorimetry (DSC) thermogram is substantially the same as shown in FIG. 8.

In some embodiments of Compound 1 freebase, the Thermogravimetric Thermal Analysis (TGA) thermogram is substantially the same as shown in FIG. 9.

TABLE 3
XRPD pattern of Compound 1 Freebase Type B

| Peak# | Diffraction angle (°, 2 θ) | Rel. Int. (%) |
|---|---|---|
| 1 | 5.4 | 5.20% |
| 2 | 5.9 | 32.20% |
| 3 | 6.3 | 2.60% |
| 4 | 6.8 | 6.30% |
| 5 | 10.5 | 8.80% |
| 6 | 12.6 | 1.20% |
| 7 | 13.7 | 30.40% |
| 8 | 14.4 | 1.40% |
| 9 | 14.8 | 7.10% |
| 10 | 15.6 | 16.90% |
| 11 | 16.7 | 100.00% |
| 12 | 18.0 | 1.90% |
| 13 | 20.5 | 7.50% |
| 14 | 21.1 | 8.30% |
| 15 | 23.3 | 9.60% |
| 16 | 24.7 | 1.80% |
| 17 | 27.3 | 3.80% |
| 18 | 30.2 | 1.00% |
| 19 | 33.8 | 0.90% |

### Compound 1 Freebase Type C

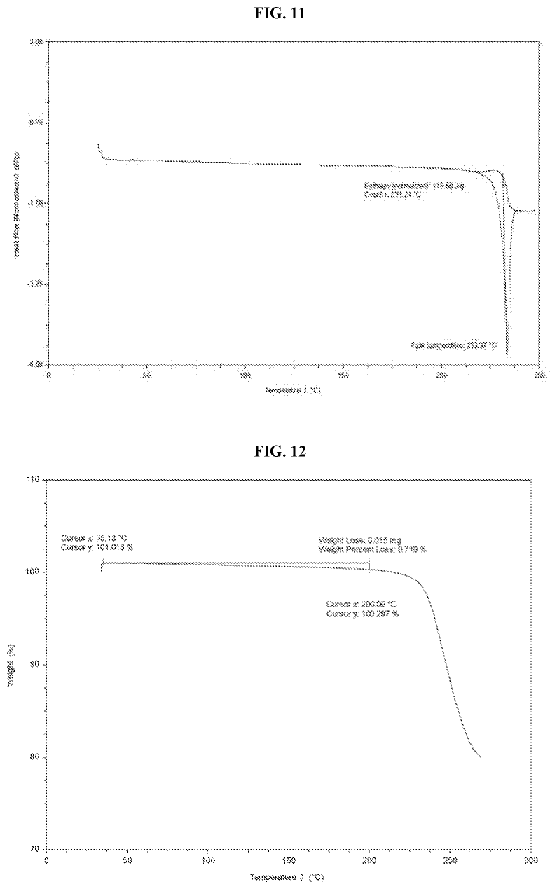

Disclosed herein is Compound 1 freebase Type C. In some embodiments, the crystalline form is Compound 1 freebase characterized as having at least one of the following properties:
(a) an X-Ray powder diffraction (XRPD) pattern substantially the same as shown in FIG. 10 as measured using Cu Kα.radiation;
(b) an X-Ray powder diffraction (XRPD) pattern with peaks at 5.2±0.2° 2θ, 6.2±0.2° 2θ, and 14.7±0.2° 2θ as measured using Cu Kα.radiation;
(c) a Differential Scanning Calorimetry (DSC) thermogram substantially the same as shown in FIG. 11;
(d) a Differential Scanning Calorimetry (DSC) thermogram with an endothermic peak having a peak temperature at about 231 [symbol] .
(e) a Thermogravimetric Thermal Analysis (TGA) thermogram substantially the same as shown in FIG. 12; or
(f) combinations thereof.

In some embodiments, the crystalline form is Compound 1 freebase characterized as having at least one of the following properties:
(a) an X-Ray powder diffraction (XRPD) pattern substantially the same as shown in FIG. 10 as measured using Cu Kα.radiation;
(b) an X-Ray powder diffraction (XRPD) pattern with peaks at 5.2±0.2° 2θ, 6.2±0.2° 2θ, and 14.7±0.2° 2θ as measured using Cu Kα.radiation;
(c) a Differential Scanning Calorimetry (DSC) thermogram substantially the same as shown in FIG. 11;
(d) a Thermogravimetric Thermal Analysis (TGA) thermogram substantially the same as shown in FIG. 12; or
(e) combinations thereof.

In some embodiments, the crystalline form is Compound 1 freebase characterized as having at least one of the following properties:
(a) an X-Ray powder diffraction (XRPD) pattern with peaks at 5.2±0.2° 2θ, 6.2±0.2° 2θ, and 14.7±0.2° 2θ as measured using Cu Kα.radiation;
(b) a Differential Scanning Calorimetry (DSC) thermogram with an endothermic peak having a peak temperature at about 231.2 [symbol] ; or
(c) combinations thereof.

In some embodiments of Compound 1 freebase, the crystalline form has an X-ray powder diffraction (XRPD) pattern substantially the same as shown in FIG. 10 as measured using Cu Kα.radiation.

In some embodiments of Compound 1 freebase, the crystalline form has an X-ray powder diffraction (XRPD) pattern with peaks found in Table 4 as measured using Cu Kα.radiation.

In some embodiments of Compound 1 freebase, the crystalline form has an X-ray powder diffraction (XRPD) pattern with peaks at 5.2±0.2° 2θ, 6.2±0.2° 2θ, and 14.7±0.2° 2θ as measured using Cu Kα.radiation.

In some embodiments of Compound 1 freebase, the X-ray powder diffraction (XRPD) pattern further comprises peaks at 13.7±0.2° 2θ, 17.9±0.2° 2θ, and 24.0±0.2° 2θ as measured using Cu Kα.radiation.

In some embodiments of Compound 1 freebase, the X-ray powder diffraction (XRPD) pattern further comprises peaks at 11.5±0.2° 2θ and 21.2±0.2° 2θ as measured using Cu Kα.radiation.

In some embodiments of Compound 1 freebase, the X-ray powder diffraction (XRPD) pattern further comprises peaks at 8.6±0.2° 2θ, 24.7±0.2° 2θ, and 28.3±0.2° 2θ as measured using Cu Kα.radiation.

In some embodiments of Compound 1 freebase, the crystalline form has an X-ray powder diffraction (XRPD) pattern with peaks at 5.2±0.2° 2θ, 6.2±0.2° 2θ, 8.6±0.2° 2θ, 11.5±0.2° 2θ, 13.7±0.2° 2θ, 14.7±0.2° 2θ, 17.9±0.2° 2θ, 21.2±0.2° 2θ, 24.0±0.2° 2θ, 24.7±0.2° 2θ, and 28.3±0.2° 2θ as measured using Cu Kα.radiation.

In some embodiments of Compound 1 freebase, the crystalline form has an X-ray powder diffraction (XRPD) pattern with at least two peaks selected from 5.2±0.2° 2θ, 6.2±0.2° 2θ, 8.6±0.2° 2θ, 11.5±0.2° 2θ, 13.7±0.2° 2θ, 14.7±0.2° 2θ, 17.9±0.2° 2θ, 21.2±0.2° 2θ, 24.0±0.2° 2θ, 24.7±0.2° 2θ, and 28.3±0.2° 2θ as measured using Cu Kα.radiation.

In some embodiments of Compound 1 freebase, the crystalline form has an X-ray powder diffraction (XRPD) pattern with at least three peaks selected from 5.2±0.2° 2θ, 6.2±0.2° 2θ, 8.6±0.2° 2θ, 11.5±0.2° 2θ, 13.7±0.2° 2θ, 14.7±0.2° 2θ, 17.9±0.2° 2θ, 21.2±0.2° 2θ, 24.0±0.2° 2θ, 24.7±0.2° 2θ, and 28.3±0.2° 2θ as measured using Cu Kα.radiation.

In some embodiments of Compound 1 freebase, the crystalline form has an X-ray powder diffraction (XRPD) pattern with at least four peaks selected from 5.2±0.2° 2θ, 6.2±0.2° 2θ, 8.6±0.2° 2θ, 11.5±0.2° 2θ, 13.7±0.2° 2θ, 14.7±0.2° 2θ, 17.9±0.2° 2θ, 21.2±0.2° 2θ, 24.0±0.2° 2θ, 24.7±0.2° 2θ, and 28.3±0.2° 2θ as measured using Cu Kα.radiation.

In some embodiments of Compound 1 freebase, the crystalline form has an X-ray powder diffraction (XRPD) pattern with at least five peaks selected from 5.2±0.2° 2θ, 6.2±0.2° 2θ, 8.6±0.2° 2θ, 11.5±0.2° 2θ, 13.7±0.2° 2θ, 14.7±0.2° 2θ, 17.9±0.2° 2θ, 21.2±0.2° 2θ, 24.0±0.2° 2θ, 24.7±0.2° 2θ, and 28.3±0.2° 2θ as measured using Cu Kα.radiation.

In some embodiments of Compound 1 freebase, the crystalline form has an X-ray powder diffraction (XRPD) pattern with at least six peaks selected from 5.2±0.2° 2θ, 6.2±0.2° 2θ, 8.6±0.2° 2θ, 11.5±0.2° 2θ, 13.7±0.2° 2θ, 14.7±0.2° 2θ, 17.9±0.2° 2θ, 21.2±0.2° 2θ, 24.0±0.2° 2θ, 24.7±0.2° 2θ, and 28.3±0.2° 2θ as measured using Cu Kα.radiation.

In some embodiments of Compound 1 freebase, the crystalline form has an X-ray powder diffraction (XRPD) pattern with at least seven peaks selected from 5.2±0.2° 2θ, 6.2±0.2° 2θ, 8.6±0.2° 2θ, 11.5±0.2° 2θ, 13.7±0.2° 2θ, 14.7±0.2° 2θ, 17.9±0.2° 2θ, 21.2±0.2° 2θ, 24.0±0.2° 2θ, 24.7±0.2° 2θ, and 28.3±0.2° 2θ as measured using Cu Kα.radiation.

In some embodiments of Compound 1 freebase, the crystalline form has an X-ray powder diffraction (XRPD) pattern with at least eight peaks selected from 5.2±0.2° 2θ, 6.2±0.2° 2θ, 8.6±0.2° 2θ, 11.5±0.2° 2θ, 13.7±0.2° 2θ, 14.7±0.2° 2θ, 17.9±0.2° 2θ, 21.2±0.2° 2θ, 24.0±0.2° 2θ, 24.7±0.2° 2θ, and 28.3±0.2° 2θ as measured using Cu Kα.radiation.

In some embodiments of Compound 1 freebase, the crystalline form has an X-ray powder diffraction (XRPD) pattern with at least nine peaks selected from 5.2±0.2° 2θ, 6.2±0.2° 2θ, 8.6±0.2° 2θ, 11.5±0.2° 2θ, 13.7±0.2° 2θ, 14.7±0.2° 2θ, 17.9±0.2° 2θ, 21.2±0.2° 2θ, 24.0±0.2° 2θ, 24.7±0.2° 2θ, and 28.3±0.2° 2θ as measured using Cu Kα.radiation.

In some embodiments of Compound 1 freebase, the crystalline form has an X-ray powder diffraction (XRPD) pattern with at least ten peaks selected from 5.2±0.2° 2θ, 6.2±0.2° 2θ, 8.6±0.2° 2θ, 11.5±0.2° 2θ, 13.7±0.2° 2θ, 14.7±0.2° 2θ, 17.9±0.2° 2θ, 21.2±0.2° 2θ, 24.0±0.2° 2θ, 24.7±0.2° 2θ, and 28.3±0.2° 2θ as measured using Cu Kα.radiation.

In some embodiments of Compound 1 freebase, the Differential Scanning Calorimetry (DSC) thermogram is substantially the same as shown in FIG. 11.

In some embodiments of Compound 1 freebase, the Differential Scanning Calorimetry (DSC) thermogram has an endothermic peak having a peak temperature at about 231.2 [symbol] .

In some embodiments of Compound 1 freebase, the Thermogravimetric Thermal Analysis (TGA) thermogram is substantially the same as shown in FIG. 12.

In some embodiments of Compound 1 freebase, the crystalline form is an anhydrate.

In some embodiments of Compound 1 freebase, the crystalline form is stable.

In some embodiments of Compound 1 freebase, the crystalline form is chemically stable.

In some embodiments of Compound 1 freebase, the crystalline form is thermodynamically stable.

In some embodiments of Compound 1 freebase, the crystalline form is more stable than Compound 1 freebase Type A, Compound 1 freebase Type B, Compound 1 freebase Type D, Compound 1 freebase Type E, or Compound 1 freebase Type F.

TABLE 4
XRPD pattern of Compound 1 Freebase Type C

| Peak# | Diffraction angle (°, 2 θ) | Rel. Int. ( %) |
|---|---|---|
| 1 | 5.2 | 100.00% |
| 2 | 6.2 | 64.10% |
| 3 | 6.8 | 5.00% |
| 4 | 8.6 | 4.80% |
| 5 | 11.1 | 3.60% |
| 6 | 11.5 | 7.40% |
| 7 | 12.6 | 5.00% |
| 8 | 13.7 | 18.80% |
| 9 | 14.7 | 44.00% |
| 10 | 17.1 | 1.60% |
| 11 | 17.3 | 1.70% |
| 12 | 17.9 | 11.40% |
| 13 | 18.4 | 3.00% |
| 14 | 18.9 | 2.80% |
| 15 | 20.2 | 1.80% |
| 16 | 20.6 | 3.50% |
| 17 | 21.2 | 6.80% |
| 18 | 22.4 | 4.40% |
| 19 | 23.2 | 4.90% |
| 20 | 24.0 | 16.00% |
| 21 | 24.7 | 7.60% |
| 22 | 25.9 | 1.20% |
| 23 | 28.3 | 6.60% |
| 24 | 29.6 | 1.60% |
| 25 | 33.0 | 1.60% |

### Compound 1 Freebase Type D

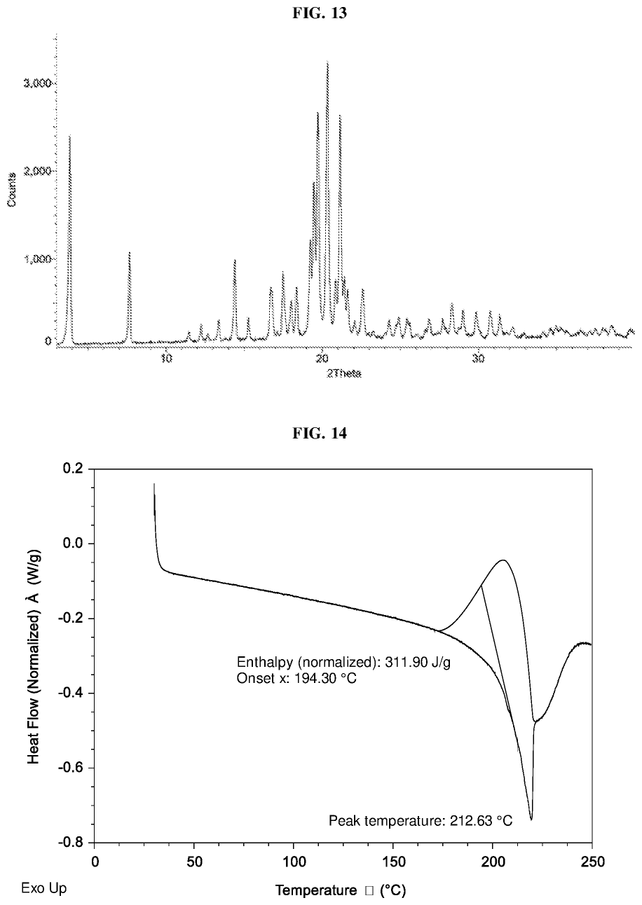

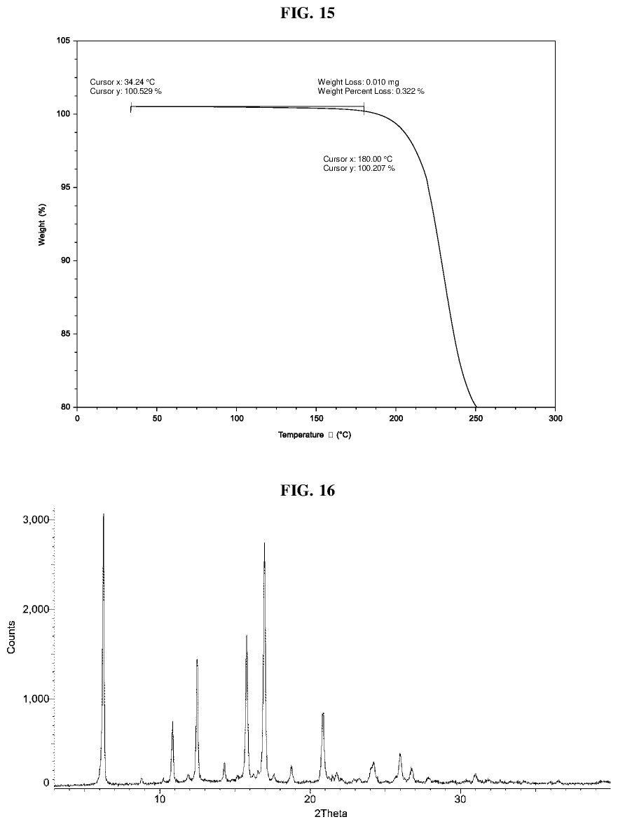

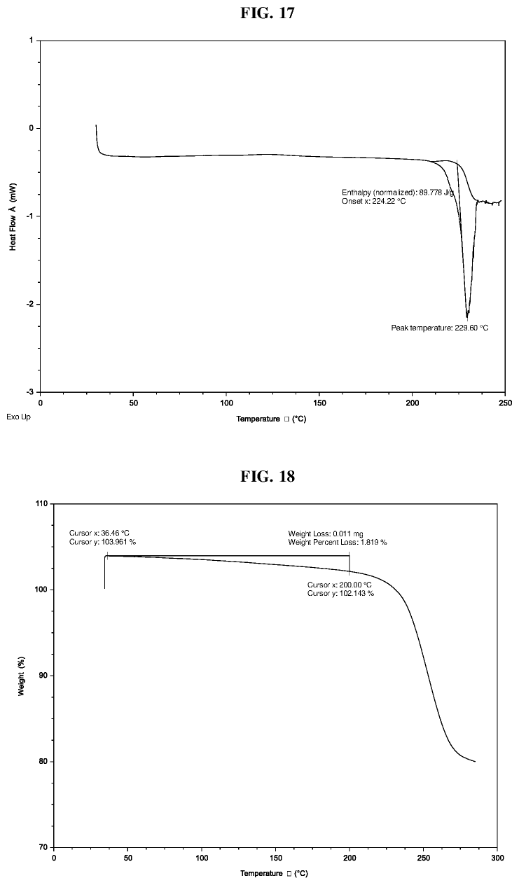

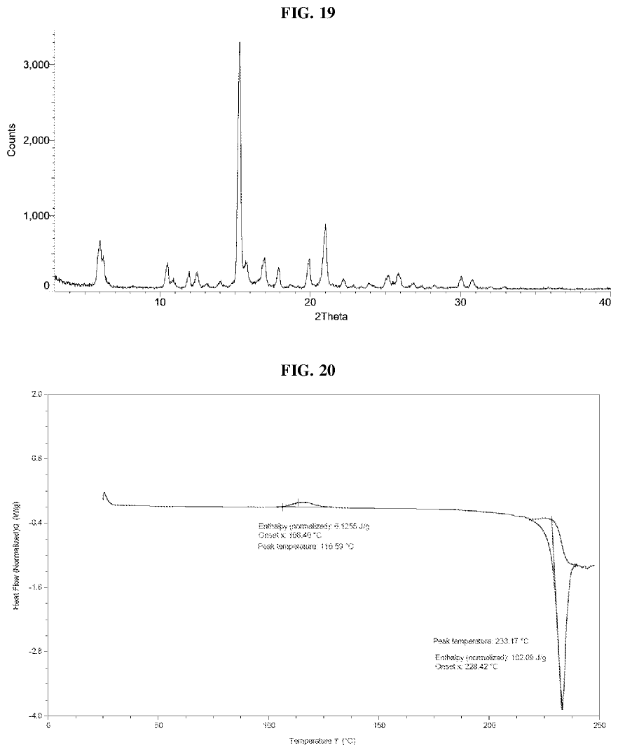

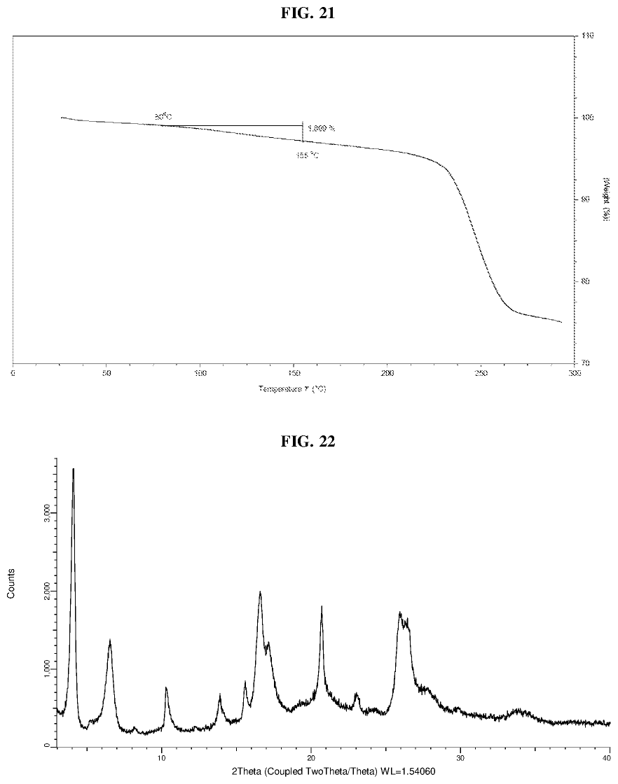

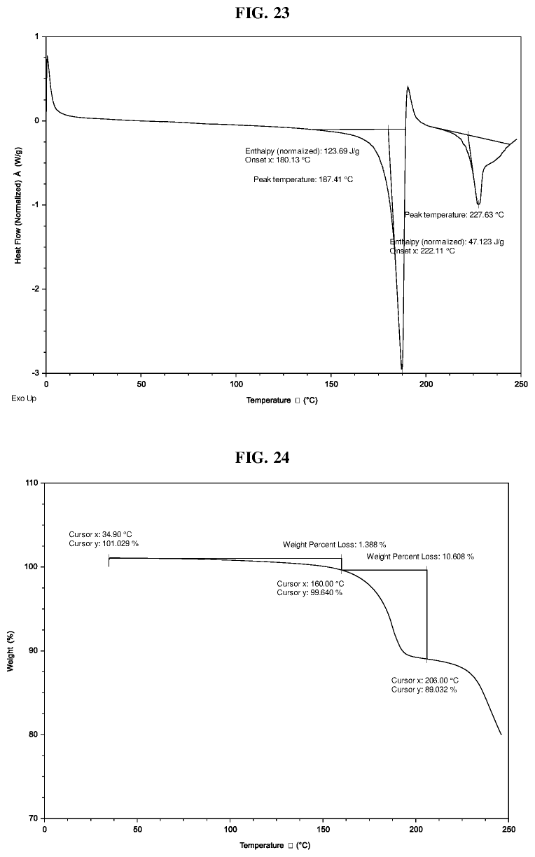

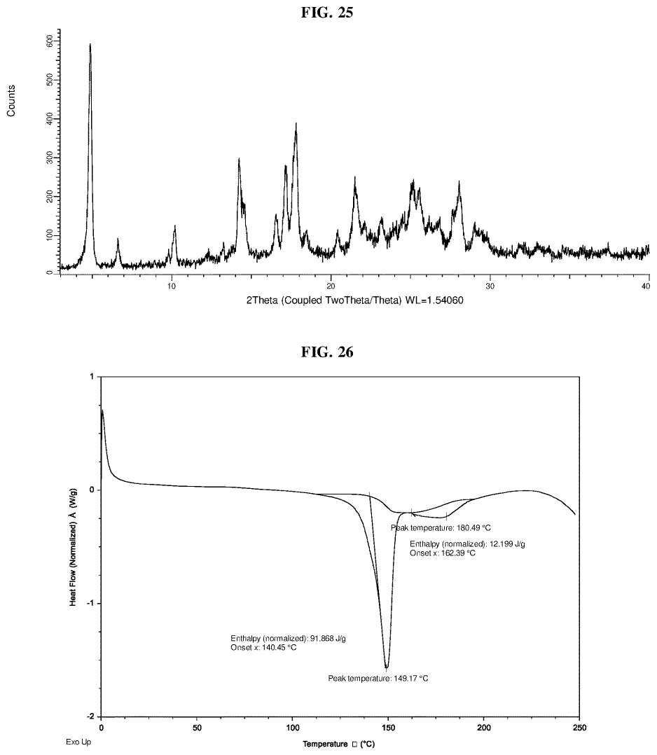

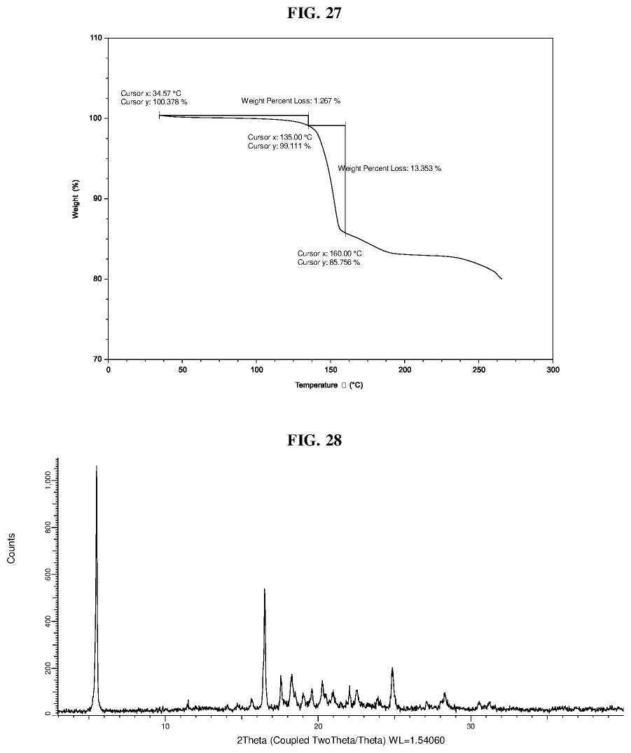

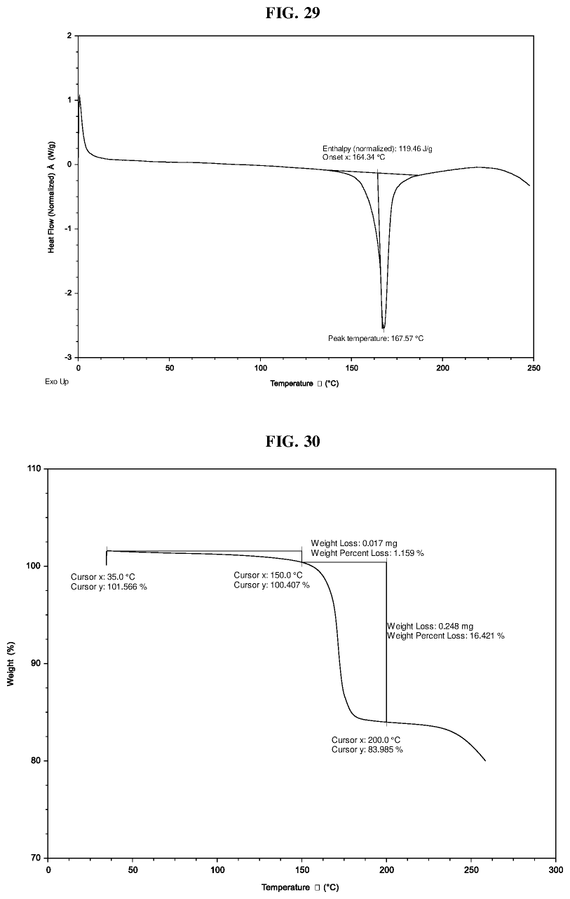

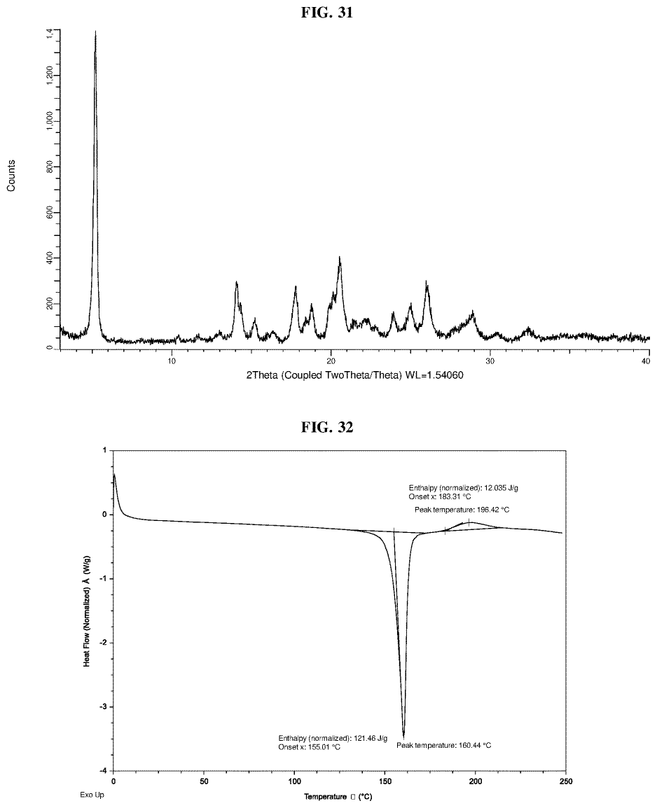

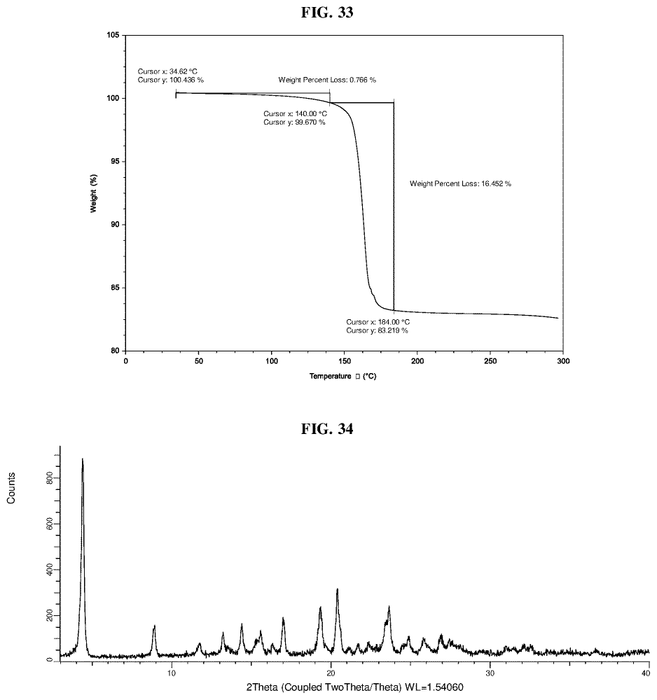

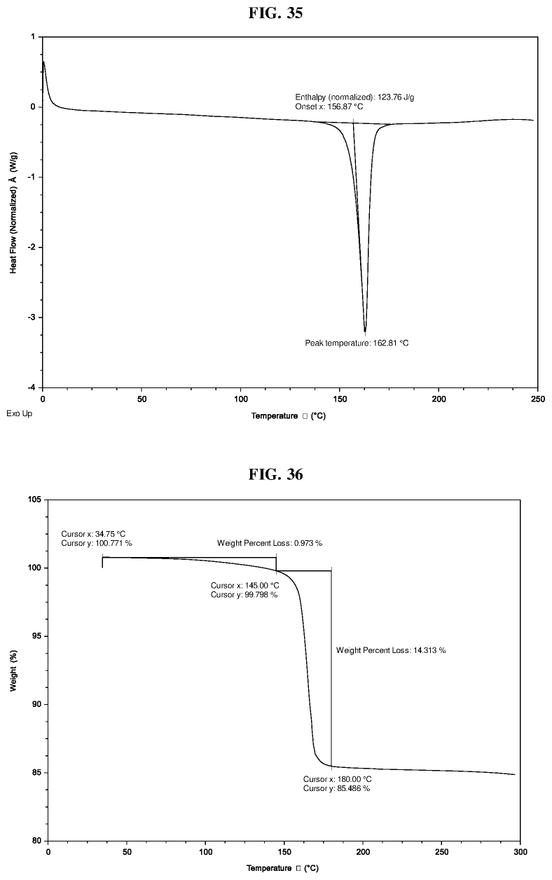

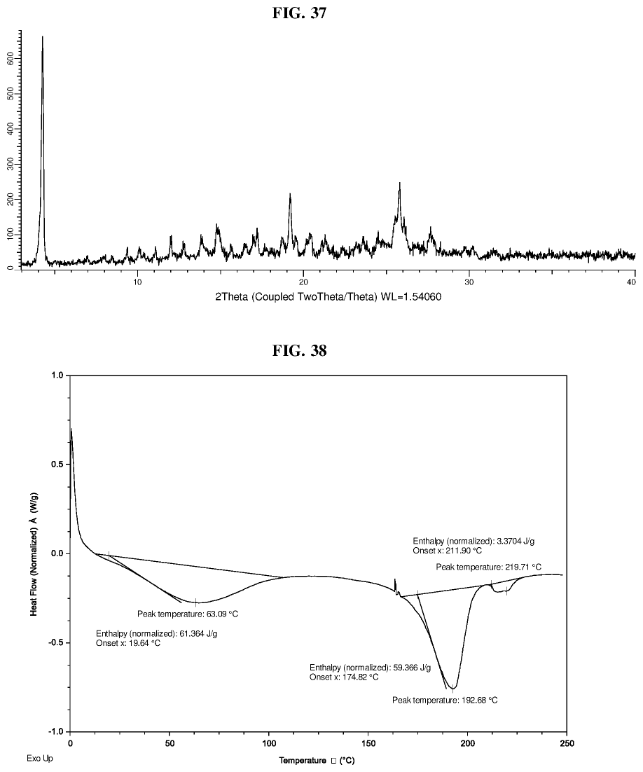

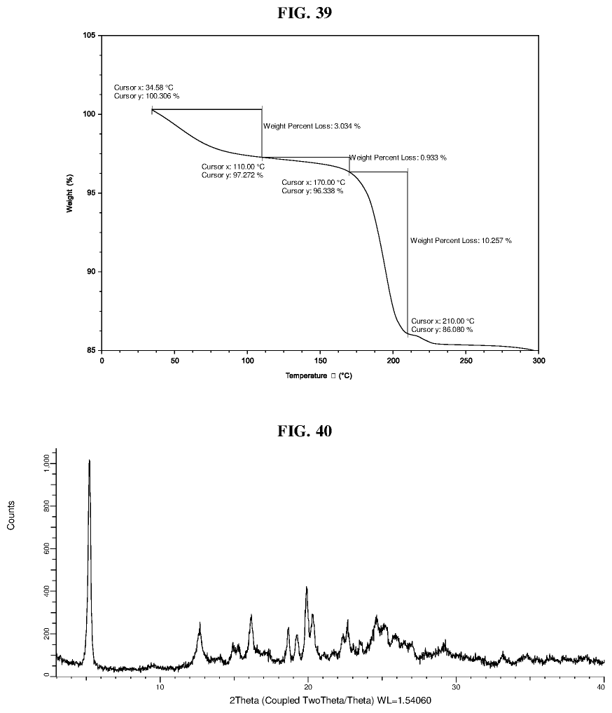

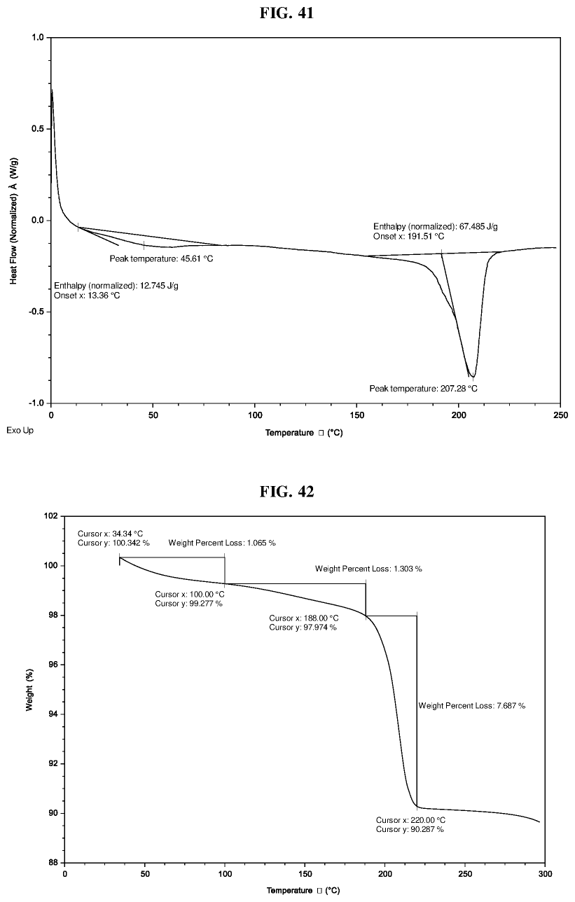

Disclosed herein is Compound 1 freebase Type D. In some embodiments, the crystalline form is Compound 1 freebase characterized as having at least one of the following properties:
(a) an X-Ray powder diffraction (XRPD) pattern substantially the same as shown in FIG. 13 as measured using Cu Kα.radiation;
(b) an X-Ray powder diffraction (XRPD) pattern with peaks at 3.8±0.2° 2θ, 20.3±0.2° 2θ, and 21.1±0.2° 2θ as measured using Cu Kα.radiation;
(c) a Differential Scanning Calorimetry (DSC) thermogram substantially the same as shown in FIG. 14;
(d) a Differential Scanning Calorimetry (DSC) thermogram with an endothermic peak having a peak temperature at about 194.3 [symbol] ;
(e) a Thermogravimetric Thermal Analysis (TGA) thermogram substantially the same as shown in FIG. 15; or
(f) combinations thereof.

In some embodiments, the crystalline form is Compound 1 freebase characterized as having at least one of the following properties:
(a) an X-Ray powder diffraction (XRPD) pattern with peaks at 3.8±0.2° 2θ, 20.3±0.2° 2θ, and 21.1±0.2° 2θ as measured using Cu Kα.radiation;
(b) a Differential Scanning Calorimetry (DSC) thermogram with an endothermic peak having a peak temperature at about 194.3 [symbol] ; or
(c) combinations thereof.

In some embodiments, the crystalline form is Compound 1 freebase characterized as having at least one of the following properties:
(a) an X-Ray powder diffraction (XRPD) pattern substantially the same as shown in FIG. 13 as measured using Cu Kα.radiation;
(b) an X-Ray powder diffraction (XRPD) pattern with peaks at 3.8±0.2° 2θ, 20.3±0.2° 2θ, and 21.1±0.2° 2θ as measured using Cu Kα.radiation;
(c) a Differential Scanning Calorimetry (DSC) thermogram substantially the same as shown in FIG. 14;
(d) a Thermogravimetric Thermal Analysis (TGA) thermogram substantially the same as shown in FIG. 15; or
(e) combinations thereof.

In some embodiments of Compound 1 freebase, the crystalline form has an X-ray powder diffraction (XRPD) pattern substantially the same as shown in FIG. 13 as measured using Cu Kα.radiation.

In some embodiments of Compound 1 freebase, the crystalline form has an X-ray powder diffraction (XRPD) pattern with peaks found in Table 5 as measured using Cu Kα.radiation.

In some embodiments of Compound 1 freebase, the crystalline form has an X-ray powder diffraction (XRPD) pattern with peaks at 3.8±0.2° 2θ, 20.3±0.2° 2θ, and 21.1±0.2° 2θ as measured using Cu Kα.radiation.

In some embodiments of Compound 1 freebase, the X-ray powder diffraction (XRPD) pattern further comprises peaks at 14.4±0.2° 2θ, 16.7±0.2° 2θ, and 19.7±0.2° 2θ as measured using Cu Kα.radiation.

In some embodiments of Compound 1 freebase, the X-ray powder diffraction (XRPD) pattern further comprises peaks at 7.7±0.2° 2θ and 15.3±0.2° 2θ as measured using Cu Kα.radiation.

In some embodiments of Compound 1 freebase, the crystalline form has an X-ray powder diffraction (XRPD) pattern with peaks at 3.8±0.2° 2θ, 7.7±0.2° 2θ, 14.4±0.2° 2θ, 15.3±0.2° 2θ, 16.7±0.2° 2θ, 19.7±0.2° 2θ, 20.3±0.2° 2θ, and 21.1±0.2° 2θ as measured using Cu Kα.radiation.

In some embodiments of Compound 1 freebase, the crystalline form has an X-ray powder diffraction (XRPD) pattern with at least two peaks selected from 3.8±0.2° 2θ, 7.7±0.2° 2θ, 14.4±0.2° 2θ, 15.3±0.2° 2θ, 16.7±0.2° 2θ, 19.7±0.2° 2θ, 20.3±0.2° 2θ, and 21.1±0.2° 2θ as measured using Cu Kα.radiation.

In some embodiments of Compound 1 freebase, the crystalline form has an X-ray powder diffraction (XRPD) pattern with at least three peaks selected from 3.8±0.2° 2θ, 7.7±0.2° 2θ, 14.4±0.2° 2θ, 15.3±0.2° 2θ, 16.7±0.2° 2θ, 19.7±0.2° 2θ, 20.3±0.2° 2θ, and 21.1±0.2° 2θ as measured using Cu Kα.radiation.

In some embodiments of Compound 1 freebase, the crystalline form has an X-ray powder diffraction (XRPD) pattern with at least four peaks selected from 3.8±0.2° 2θ, 7.7±0.2° 2θ, 14.4±0.2° 2θ, 15.3±0.2° 2θ, 16.7±0.2° 2θ, 19.7±0.2° 2θ, 20.3±0.2° 2θ, and 21.1±0.2° 2θ as measured using Cu Kα.radiation.

In some embodiments of Compound 1 freebase, the crystalline form has an X-ray powder diffraction (XRPD) pattern with at least five peaks selected from 3.8±0.2° 2θ, 7.7±0.2° 2θ, 14.4±0.2° 2θ, 15.3±0.2° 2θ, 16.7±0.2° 2θ, 19.7±0.2° 2θ, 20.3±0.2° 2θ, and 21.1±0.2° 2θ as measured using Cu Kα.radiation.

In some embodiments of Compound 1 freebase, the crystalline form has an X-ray powder diffraction (XRPD) pattern with at least six peaks selected from 3.8±0.2° 2θ, 7.7±0.2° 2θ, 14.4±0.2° 2θ, 15.3±0.2° 2θ, 16.7±0.2° 2θ, 19.7±0.2° 2θ, 20.3±0.2° 2θ, and 21.1±0.2° 2θ as measured using Cu Kα.radiation.

In some embodiments of Compound 1 freebase, the crystalline form has an X-ray powder diffraction (XRPD) pattern with at least seven peaks selected from 3.8±0.2° 2θ, 7.7±0.2° 2θ, 14.4±0.2° 2θ, 15.3±0.2° 2θ, 16.7±0.2° 2θ, 19.7±0.2° 2θ, 20.3±0.2° 2θ, and 21.1±0.2° 2θ as measured using Cu Kα.radiation.

In some embodiments of Compound 1 freebase, the Differential Scanning Calorimetry (DSC) thermogram is substantially the same as shown in FIG. 14.

In some embodiments of Compound 1 freebase, the Differential Scanning Calorimetry (DSC) thermogram has an endothermic peak having a peak temperature at about 194.3 [symbol] .

In some embodiments of Compound 1 freebase, the Thermogravimetric Thermal Analysis (TGA) thermogram is substantially the same as shown in FIG. 15.

In some embodiments of Compound 1 freebase, the crystalline form is an anhydrate.

TABLE 5
XRPD pattern of Compound 1 Freebase Type D

| Peak# | Diffraction angle (°, 2 θ) | Rel. Int. (%) |
|---|---|---|
| 1 | 3.8 | 66.90% |
| 2 | 7.7 | 33.50% |
| 3 | 11.5 | 3.60% |
| 4 | 12.2 | 5.70% |
| 5 | 12.7 | 2.10% |
| 6 | 13.4 | 7.00% |
| 7 | 14.4 | 29.40% |
| 8 | 15.3 | 9.00% |
| 9 | 16.7 | 18.30% |
| 10 | 17.5 | 24.90% |
| 11 | 18.0 | 13.20% |
| 12 | 18.4 | 18.00% |
| 13 | 19.2 | 33.80% |
| 14 | 19.5 | 57.00% |
| 15 | 19.7 | 81.60% |
| 16 | 20.3 | 100.00% |
| 17 | 20.9 | 20.70% |
| 18 | 21.1 | 83.50% |
| 19 | 21.4 | 18.60% |
| 20 | 21.6 | 16.80% |
| 21 | 22.1 | 5.10% |
| 22 | 22.6 | 17.90% |
| 23 | 23.3 | 2.30% |
| 24 | 24.0 | 1.50% |
| 25 | 24.3 | 7.50% |
| 26 | 25.0 | 7.60% |
| 27 | 25.4 | 7.10% |
| 28 | 26.1 | 1.30% |
| 29 | 26.6 | 2.70% |
| 30 | 26.8 | 7.20% |
| 31 | 27.7 | 4.30% |
| 32 | 28.3 | 12.40% |
| 33 | 29.0 | 10.00% |
| 34 | 29.9 | 9.70% |
| 35 | 30.7 | 11.80% |
| 36 | 31.4 | 9.50% |
| 37 | 32.2 | 3.90% |
| 38 | 32.9 | 1.90% |
| 39 | 34.1 | 2.60% |
| 40 | 34.6 | 2.20% |
| 41 | 35.0 | 3.80% |
| 42 | 35.3 | 3.50% |
| 43 | 35.6 | 2.30% |
| 44 | 36.5 | 2.90% |
| 45 | 37.1 | 1.40% |
| 46 | 37.5 | 2.30% |
| 47 | 38.0 | 1.50% |
| 48 | 38.5 | 3.60% |
| 49 | 39.7 | 2.80% |

### Compound 1 Freebase Type E

Disclosed herein is Compound 1 freebase Type E. In some embodiments, the crystalline form is Compound 1 freebase characterized as having at least one of the following properties:
(a) an X-Ray powder diffraction (XRPD) pattern substantially the same as shown in FIG. 16 as measured using Cu Kα.radiation;
(b) an X-Ray powder diffraction (XRPD) pattern with peaks at 6.3±0.2° 2θ, 12.5±0.2° 2θ, and 17.0±0.2° 2θ as measured using Cu Kα.radiation;
(c) a Differential Scanning Calorimetry (DSC) thermogram substantially the same as shown in FIG. 17;
(d) a Differential Scanning Calorimetry (DSC) thermogram with an endothermic peak having a peak temperature at about 224.2 [symbol] ;
(e) a Thermogravimetric Thermal Analysis (TGA) thermogram substantially the same as shown in FIG. 18; or
(f) combinations thereof.

In some embodiments, the crystalline form is Compound 1 freebase characterized as having at least one of the following properties:
(a) an X-Ray powder diffraction (XRPD) pattern substantially the same as shown in FIG. 16 as measured using Cu Kα.radiation;
(b) an X-Ray powder diffraction (XRPD) pattern with peaks at 6.3±0.2° 2θ, 12.5±0.2° 2θ, and 17.0±0.2° 2θ as measured using Cu Kα.radiation;
(c) a Differential Scanning Calorimetry (DSC) thermogram substantially the same as shown in FIG. 17;
(d) a Thermogravimetric Thermal Analysis (TGA) thermogram substantially the same as shown in FIG. 18; or
(e) combinations thereof.

In some embodiments, the crystalline form is Compound 1 freebase characterized as having at least one of the following properties:
(a) an X-Ray powder diffraction (XRPD) pattern with peaks at 6.3±0.2° 2θ, 12.5±0.2° 2θ, and 17.0±0.2° 2θ as measured using Cu Kα.radiation;
(b) a Differential Scanning Calorimetry (DSC) thermogram with an endothermic peak having a peak temperature at about 224.4 [symbol] ; or
(c) combinations thereof.

In some embodiments of Compound 1 freebase, the crystalline form has an X-ray powder diffraction (XRPD) pattern substantially the same as shown in FIG. 16 as measured using Cu Kα.radiation.

In some embodiments of Compound 1 freebase, the crystalline form has an X-ray powder diffraction (XRPD) pattern with peaks found in Table 6 as measured using Cu Kα.radiation.

In some embodiments of Compound 1 freebase, the crystalline form has an X-ray powder diffraction (XRPD) pattern with peaks at 6.3±0.2° 2θ, 12.5±0.2° 2θ, and 17.0±0.2° 2θ as measured using Cu Kα.radiation.

In some embodiments of Compound 1 freebase, the X-ray powder diffraction (XRPD) pattern further comprises peaks at 10.9±0.2° 2θ, 15.8±0.2° 2θ, and 20.9±0.2° 2θ as measured using Cu Kα.radiation.

In some embodiments of Compound 1 freebase, the X-ray powder diffraction (XRPD) pattern further comprises peaks at 14.3±0.2° 2θ and 26.0±0.2° 2θ as measured using Cu Kα.radiation.

In some embodiments of Compound 1 freebase, the crystalline form has an X-ray powder diffraction (XRPD) pattern with peaks at 6.3±0.2° 2θ, 10.9±0.2° 2θ, 12.5±0.2° 2θ, 14.3±0.2° 2θ, 15.8±0.2° 2θ, 17.0±0.2° 2θ, 20.9±0.2° 2θ, and 26.0±0.2° 2θ as measured using Cu Kα.radiation.

In some embodiments of Compound 1 freebase, the crystalline form has an X-ray powder diffraction (XRPD) pattern with at least two peaks selected from 6.3±0.2° 2θ, 10.9±0.2° 2θ, 12.5±0.2° 2θ, 14.3±0.2° 2θ, 15.8±0.2° 2θ, 17.0±0.2° 2θ, 20.9±0.2° 2θ, and 26.0±0.2° 2θ as measured using Cu Kα.radiation.

In some embodiments of Compound 1 freebase, the crystalline form has an X-ray powder diffraction (XRPD) pattern with at least three peaks selected from 6.3±0.2° 2θ, 10.9±0.2° 2θ, 12.5±0.2° 2θ, 14.3±0.2° 2θ, 15.8±0.2° 2θ, 17.0±0.2° 2θ, 20.9±0.2° 2θ, and 26.0±0.2° 2θ as measured using Cu Kα.radiation.

In some embodiments of Compound 1 freebase, the crystalline form has an X-ray powder diffraction (XRPD) pattern with at least four peaks selected from 6.3±0.2° 2θ, 10.9±0.2° 2θ, 12.5±0.2° 2θ, 14.3±0.2° 2θ, 15.8±0.2° 2θ, 17.0±0.2° 2θ, 20.9±0.2° 2θ, and 26.0±0.2° 2θ as measured using Cu Kα.radiation.

In some embodiments of Compound 1 freebase, the crystalline form has an X-ray powder diffraction (XRPD) pattern with at least five peaks selected from 6.3±0.2° 2θ, 10.9±0.2° 2θ, 12.5±0.2° 2θ, 14.3±0.2° 2θ, 15.8±0.2° 2θ, 17.0±0.2° 2θ, 20.9±0.2° 2θ, and 26.0±0.2° 2θ as measured using Cu Kα.radiation.

In some embodiments of Compound 1 freebase, the crystalline form has an X-ray powder diffraction (XRPD) pattern with at least six peaks selected from 6.3±0.2° 2θ, 10.9±0.2° 2θ, 12.5±0.2° 2θ, 14.3±0.2° 2θ, 15.8±0.2° 2θ, 17.0±0.2° 2θ, 20.9±0.2° 2θ, and 26.0±0.2° 2θ as measured using Cu Kα.radiation.

In some embodiments of Compound 1 freebase, the crystalline form has an X-ray powder diffraction (XRPD) pattern with at least seven peaks selected from 6.3±0.2° 2θ, 10.9±0.2° 2θ, 12.5±0.2° 2θ, 14.3±0.2° 2θ, 15.8±0.2° 2θ, 17.0±0.2° 2θ, 20.9±0.2° 2θ, and 26.0±0.2° 2θ as measured using Cu Kα.radiation.

In some embodiments of Compound 1 freebase, the Differential Scanning Calorimetry (DSC) thermogram is substantially the same as shown in FIG. 17.

In some embodiments of Compound 1 freebase, the Differential Scanning Calorimetry (DSC) thermogram has an endothermic peak having a peak temperature at about 224.2 [symbol] .

In some embodiments of Compound 1 freebase, the Thermogravimetric Thermal Analysis (TGA) thermogram is substantially the same as shown in FIG. 18.

In some embodiments of Compound 1 freebase, the crystalline form is an anhydrate.

TABLE 6
XRPD pattern of Compound 1 Freebase Type E

| Peak# | Diffraction angle (°, 2 θ) | Rel. Int. (%) |
|---|---|---|
| 1 | 6.3 | 100.00% |
| 2 | 8.8 | 1.90% |
| 3 | 10.3 | 1.70% |
| 4 | 10.9 | 23.80% |
| 5 | 11.9 | 2.50% |
| 6 | 12.5 | 48.10% |
| 7 | 14.3 | 7.40% |
| 8 | 15.2 | 1.20% |
| 9 | 15.8 | 57.50% |
| 10 | 16.2 | 2.40% |
| 11 | 16.6 | 3.50% |
| 12 | 17.0 | 90.30% |
| 13 | 17.6 | 3.20% |
| 14 | 18.8 | 6.70% |
| 15 | 20.9 | 25.70% |
| 16 | 21.5 | 1.90% |
| 17 | 21.8 | 4.20% |
| 18 | 22.9 | 1.70% |
| 19 | 23.3 | 1.30% |
| 20 | 24.1 | 6.80% |
| 21 | 25.8 | 2.40% |
| 22 | 26.0 | 10.30% |
| 23 | 26.7 | 5.00% |
| 24 | 27.9 | 2.40% |
| 25 | 31.0 | 3.70% |
| 26 | 32.6 | 1.70% |
| 27 | 33.3 | 1.10% |
| 28 | 34.2 | 0.80% |
| 29 | 36.0 | 1.10% |
| 30 | 36.5 | 1.00% |

### Compound 1 Freebase Type F

Disclosed herein is Compound 1 freebase Type F. In some embodiments, the crystalline form is Compound 1 freebase characterized as having at least one of the following properties:
(a) an X-Ray powder diffraction (XRPD) pattern substantially the same as shown in FIG. 19 as measured using Cu Kα.radiation;
(b) an X-Ray powder diffraction (XRPD) pattern with peaks at 6.0±0.2° 2θ, 15.3±0.2° 2θ, and 21.0±0.2° 2θ as measured using Cu Kα.radiation;
(c) a Differential Scanning Calorimetry (DSC) thermogram substantially the same as shown in FIG. 20;
(d) a Differential Scanning Calorimetry (DSC) thermogram with an endothermic peak having a peak temperature at about 228.4 [symbol] ;
(e) a Differential Scanning Calorimetry (DSC) thermogram with an exothermic peak having a peak temperature at about 106.4 [symbol] ;
(f) a Thermogravimetric Thermal Analysis (TGA) thermogram substantially the same as shown in FIG. 21; or
(g) combinations thereof.

In some embodiments, the crystalline form is Compound 1 freebase characterized as having at least one of the following properties:
(a) an X-Ray powder diffraction (XRPD) pattern substantially the same as shown in FIG. 19 as measured using Cu Kα.radiation;
(b) an X-Ray powder diffraction (XRPD) pattern with peaks at 6.0±0.2° 2θ, 15.3±0.2° 2θ, and 21.0±0.2° 2θ as measured using Cu Kα.radiation;
(c) a Differential Scanning Calorimetry (DSC) thermogram substantially the same as shown in FIG. 20;
(d) a Thermogravimetric Thermal Analysis (TGA) thermogram substantially the same as shown in FIG. 21; or
(e) combinations thereof.

In some embodiments, the crystalline form is Compound 1 freebase characterized as having at least one of the following properties:
(a) an X-Ray powder diffraction (XRPD) pattern with peaks at 6.0±0.2° 2θ, 15.3±0.2° 2θ, and 21.0±0.2° 2θ as measured using Cu Kα.radiation;
(b) a Differential Scanning Calorimetry (DSC) thermogram with an endothermic peak having a peak temperature at about 228.4 [symbol] ;
(c) a Differential Scanning Calorimetry (DSC) thermogram with an exothermic peak having a peak temperature at about 106.4 t; or
(d) combinations thereof.

In some embodiments of Compound 1 freebase, the crystalline form has an X-ray powder diffraction (XRPD) pattern substantially the same as shown in FIG. 19 as measured using Cu Kα.radiation.

In some embodiments of Compound 1 freebase, the crystalline form has an X-ray powder diffraction (XRPD) pattern with peaks found in Table 7 as measured using Cu Kα.radiation.

In some embodiments of Compound 1 freebase, the crystalline form has an X-ray powder diffraction (XRPD) pattern with peaks at 6.0±0.2° 2θ, 15.3±0.2° 2θ, and 21.0±0.2° 2θ as measured using Cu Kα.radiation.

In some embodiments of Compound 1 freebase, the X-ray powder diffraction (XRPD) pattern further comprises peaks at 10.5±0.2° 2θ, 17.9±0.2° 2θ, and 19.9±0.2° 2θ as measured using Cu Kα.radiation.

In some embodiments of Compound 1 freebase, the X-ray powder diffraction (XRPD) pattern further comprises peaks at 11.9±0.2° 2θ, 25.2±0.2° 2θ, and 25.9±0.2° 2θ as measured using Cu Kα.radiation.

In some embodiments of Compound 1 freebase, the crystalline form has an X-ray powder diffraction (XRPD) pattern with peaks at 6.0±0.2° 2θ, 10.5±0.2° 2θ, 11.9±0.2° 2θ, 15.3±0.2° 2θ, 17.9±0.2° 2θ, 19.9±0.2° 2θ, 21.0±0.2° 2θ, 25.2±0.2° 2θ, and 25.9±0.2° 2θ as measured using Cu Kα.radiation.

In some embodiments of Compound 1 freebase, the crystalline form has an X-ray powder diffraction (XRPD) pattern with at least two peaks selected from 6.0±0.2° 2θ, 10.5±0.2° 2θ, 11.9±0.2° 2θ, 15.3±0.2° 2θ, 17.9±0.2° 2θ, 19.9±0.2° 2θ, 21.0±0.2° 2θ, 25.2±0.2° 2θ, and 25.9±0.2° 2θ as measured using Cu Kα.radiation.

In some embodiments of Compound 1 freebase, the crystalline form has an X-ray powder diffraction (XRPD) pattern with at least three peaks selected from 6.0±0.2° 2θ, 10.5±0.2° 2θ, 11.9±0.2° 2θ, 15.3±0.2° 2θ, 17.9±0.2° 2θ, 19.9±0.2° 2θ, 21.0±0.2° 2θ, 25.2±0.2° 2θ, and 25.9±0.2° 2θ as measured using Cu Kα.radiation.

In some embodiments of Compound 1 freebase, the crystalline form has an X-ray powder diffraction (XRPD) pattern with at least four peaks selected from 6.0±0.2° 2θ, 10.5±0.2° 2θ, 11.9±0.2° 2θ, 15.3±0.2° 2θ, 17.9±0.2° 2θ, 19.9±0.2° 2θ, 21.0±0.2° 2θ, 25.2±0.2° 2θ, and 25.9±0.2° 2θ as measured using Cu Kα.radiation.

In some embodiments of Compound 1 freebase, the crystalline form has an X-ray powder diffraction (XRPD) pattern with at least five peaks selected from 6.0±0.2° 2θ, 10.5±0.2° 2θ, 11.9±0.2° 2θ, 15.3±0.2° 2θ, 17.9±0.2° 2θ, 19.9±0.2° 2θ, 21.0±0.2° 2θ, 25.2±0.2° 2θ, and 25.9±0.2° 2θ as measured using Cu Kα.radiation.

In some embodiments of Compound 1 freebase, the crystalline form has an X-ray powder diffraction (XRPD) pattern with at least six peaks selected from 6.0±0.2° 2θ, 10.5±0.2° 2θ, 11.9±0.2° 2θ, 15.3±0.2° 2θ, 17.9±0.2° 2θ, 19.9±0.2° 2θ, 21.0±0.2° 2θ, 25.2±0.2° 2θ, and 25.9±0.2° 2θ as measured using Cu Kα.radiation.

In some embodiments of Compound 1 freebase, the crystalline form has an X-ray powder diffraction (XRPD) pattern with at least seven peaks selected from 6.0±0.2° 2θ, 10.5±0.2° 2θ, 11.9±0.2° 2θ, 15.3±0.2° 2θ, 17.9±0.2° 2θ, 19.9±0.2° 2θ, 21.0±0.2° 2θ, 25.2±0.2° 2θ, and 25.9±0.2° 2θ as measured using Cu Kα.radiation.

In some embodiments of Compound 1 freebase, the crystalline form has an X-ray powder diffraction (XRPD) pattern with at least eight peaks selected from 6.0±0.2° 2θ, 10.5±0.2° 2θ, 11.9±0.2° 2θ, 15.3±0.2° 2θ, 17.9±0.2° 2θ, 19.9±0.2° 2θ, 21.0±0.2° 2θ, 252±0.2° 2θ, and 25.9±0.2° 2θ as measured using Cu Kα.radiation.

In some embodiments of Compound 1 freebase, the Differential Scanning Calorimetry (DSC) thermogram is substantially the same as shown in FIG. 20.

In some embodiments of Compound 1 freebase, the Differential Scanning Calorimetry (DSC) thermogram has an endothermic peak having a peak temperature at about 228.4 [symbol] .

In some embodiments of Compound 1 freebase, the Differential Scanning Calorimetry (DSC) thermogram has an exothermic peak having a peak temperature at about 106.4 [symbol] .

In some embodiments of Compound 1 freebase, the Thermogravimetric Thermal Analysis (TGA) thermogram is substantially the same as shown in FIG. 21.

In some embodiments of Compound 1 freebase, the crystalline form is an anhydrate.

TABLE 7
XRPD pattern of Compound 1 Freebase Type F

| | Diffraction angle | Rel. Int. |
|---|---|---|
| Peak# | ( [symbol] , 2 θ) | (%) |
| 1 | 6.0 | 18.60% |
| 2 | 6.3 | 9.80% |
| 3 | 10.5 | 8.70% |
| 4 | 10.8 | 3.00% |
| 5 | 11.9 | 6.30% |
| 6 | 12.5 | 6.30% |
| 7 | 13.1 | 1.20% |
| 8 | 14.0 | 2.20% |
| 9 | 15.3 | 100.00% |
| 10 | 15.7 | 9.60% |
| 11 | 17.0 | 11.70% |
| 12 | 17.9 | 8.00% |
| 13 | 18.7 | 1.60% |
| 14 | 19.9 | 11.70% |
| 15 | 21.0 | 25.30% |
| 16 | 22.2 | 3.40% |
| 17 | 22.8 | 1.00% |
| 18 | 23.4 | 0.70% |
| 19 | 23.9 | 1.60% |
| 20 | 25.2 | 4.90% |
| 21 | 25.9 | 5.80% |
| 22 | 26.9 | 1.70% |
| 23 | 27.4 | 0.60% |
| 24 | 28.3 | 1.10% |
| 25 | 30.1 | 4.50% |
| 26 | 30.8 | 3.40% |
| 27 | 32.0 | 0.60% |
| 28 | 32.9 | 1.00% |
| 29 | 38.0 | 0.40% |

### Compound 1 HC Salt Type A

Disclosed herein is Compound 1 HCl salt Type A. In some embodiments, the crystalline form is Compound 1 HCl salt characterized as having at least one of the following properties:
(a) an X-Ray powder diffraction (XRPD) pattern substantially the same as shown in FIG. 22 as measured using Cu Kα.radiation;
(b) an X-Ray powder diffraction (XRPD) pattern with peaks at 4.1±0.2° 2θ, 10.3±0.2° 2θ, and 16.6±0.2° 2θ as measured using Cu Kα.radiation;
(c) a Differential Scanning Calorimetry (DSC) thermogram substantially the same as shown in FIG. 23;
(d) a Thermogravimetric Thermal Analysis (TGA) thermogram substantially the same as shown in FIG. 24; or
(e) combinations thereof.

In some embodiments of Compound 1 HCl salt, the crystalline form has an X-ray powder diffraction (XRPD) pattern substantially the same as shown in FIG. 22 as measured using Cu Kα.radiation.

In some embodiments of Compound 1 HCl salt, the crystalline form has an X-ray powder diffraction (XRPD) pattern with peaks found in Table 8 as measured using Cu Kα.radiation.

In some embodiments of Compound 1 HCl salt, the crystalline form has an X-ray powder diffraction (XRPD) pattern with peaks at 4.1±0.2° 2θ, 10.3±0.2° 2θ, and 16.6±0.2° 2θ as measured using Cu Kα.radiation.

In some embodiments of Compound 1 HCl salt, the X-ray powder diffraction (XRPD) pattern further comprises peaks at 6.6±0.2° 2θ, 13.8±0.2° 2θ, and 20.8±0.2° 2θ as measured using Cu Kα.radiation.

In some embodiments of Compound 1 HCl salt, the X-ray powder diffraction (XRPD) pattern further comprises peaks at 26.0±0.2° 2θ and 26.4±0.2° 2θ as measured using Cu Kα.radiation.

In some embodiments of Compound 1 HCl salt, the crystalline form has an X-ray powder diffraction (XRPD) pattern with peaks at 4.1±0.2° 2θ, 6.6±0.2° 2θ, 10.3±0.2° 2θ, 13.8±0.2° 2θ, 16.6±0.2° 2θ, 20.8±0.2° 2θ, 26.0±0.2° 2θ, and 26.4±0.2° 2θ as measured using Cu Kα.radiation.

In some embodiments of Compound 1 HCl salt, the crystalline form has an X-ray powder diffraction (XRPD) pattern with at least two peaks selected from 4.1±0.2° 2θ, 6.6±0.2° 2θ, 10.3±0.2° 2θ, 13.8±0.2° 2θ, 16.6±0.2° 2θ, 20.8±0.2° 2θ, 26.0±0.2° 2θ, and 26.4±0.2° 2θ as measured using Cu Kα.radiation.

In some embodiments of Compound 1 HCl salt, the crystalline form has an X-ray powder diffraction (XRPD) pattern with at least three peaks selected from 4.1±0.2° 2θ, 6.6±0.2° 2θ, 10.3±0.2° 2θ, 13.8±0.2° 2θ, 16.6±0.2° 2θ, 20.8±0.2° 2θ, 26.0±0.2° 2θ, and 26.4±0.2° 2θ as measured using Cu Kα.radiation.

In some embodiments of Compound 1 HCl salt, the crystalline form has an X-ray powder diffraction (XRPD) pattern with at least four peaks selected from 4.1±0.2° 2θ, 6.6±0.2° 2θ, 10.3±0.2° 2θ, 13.8±0.2° 2θ, 16.6±0.2° 2θ, 20.8±0.2° 2θ, 26.0±0.2° 2θ, and 26.4±0.2° 2θ as measured using Cu Kα.radiation.

In some embodiments of Compound 1 HCl salt, the crystalline form has an X-ray powder diffraction (XRPD) pattern with at least five peaks selected from 4.1±0.2° 2θ, 6.6±0.2° 2θ, 10.3±0.2° 2θ, 13.8±0.2° 2θ, 16.6±0.2° 2θ, 20.8±0.2° 2θ, 26.0±0.2° 2θ, and 26.4±0.2° 2θ as measured using Cu Kα.radiation.

In some embodiments of Compound 1 HCl salt, the crystalline form has an X-ray powder diffraction (XRPD) pattern with at least six peaks selected from 4.1±0.2° 2θ, 6.6±0.2° 2θ, 10.3±0.2° 2θ, 13.8±0.2° 2θ, 16.6±0.2° 2θ, 20.8±0.2° 2θ, 26.0±0.2° 2θ, and 26.4±0.2° 2θ as measured using Cu Kα.radiation.

In some embodiments of Compound 1 HCl salt, the crystalline form has an X-ray powder diffraction (XRPD) pattern with at least seven peaks selected from 4.1±0.2° 2θ, 6.6±0.2° 2θ, 10.3±0.2° 2θ, 13.8±0.2° 2θ, 16.6±0.2° 2θ, 20.8±0.2° 2θ, 26.0±0.2° 2θ, and 26.4±0.2° 2θ as measured using Cu Kα.radiation.

In some embodiments of Compound 1 HCl salt, the crystalline form has an X-ray powder diffraction (XRPD) pattern with at least eight peaks selected from 4.1±0.2° 2θ, 6.6±0.2° 2θ, 10.3±0.2° 2θ, 13.8±0.2° 2θ, 16.6±0.2° 2θ, 20.8±0.2° 2θ, 26.0±0.2° 2θ, and 26.4±0.2° 2θ as measured using Cu Kα.radiation.

In some embodiments of Compound 1 HCl salt, the Differential Scanning Calorimetry (DSC) thermogram is substantially the same as shown in FIG. 23.

In some embodiments of Compound 1 HCl salt, the Thermogravimetric Thermal Analysis (TGA) thermogram is substantially the same as shown in FIG. 24.

In some embodiments of Compound 1 HCl salt, the crystalline form is an anhydrate.

TABLE 8
XRPD peaks table of Compound 1 HCl salt Type A

| | Diffraction angle | Rel. Int. |
|---|---|---|
| Peak# | ( [symbol] , 2 θ) | (%) |
| 1 | 4.1 | 100.00% |
| 2 | 6.6 | 33.90% |
| 3 | 10.3 | 20.70% |
| 4 | 13.8 | 17.60% |
| 5 | 15.6 | 26.50% |
| 6 | 16.6 | 66.90% |
| 7 | 17.2 | 47.50% |
| 8 | 20.8 | 59.20% |
| 9 | 23.1 | 16.10% |
| 10 | 26.0 | 71.90% |
| 11 | 26.4 | 67.80% |

### Compound 1 Sulfate Salt Type A

Disclosed herein is Compound 1 Sulfate salt Type A. In some embodiments, the crystalline form is Compound 1 Sulfate salt characterized as having at least one of the following properties:
(a) an X-Ray powder diffraction (XRPD) pattern substantially the same as shown in FIG. 25 as measured using Cu Kα.radiation;
(b) an X-Ray powder diffraction (XRPD) pattern with peaks at 4.9±0.2° 2θ, 17.2±0.2° 2θ, and 21.5±0.2° 2θ as measured using Cu Kα.radiation;
(c) a Differential Scanning Calorimetry (DSC) thermogram substantially the same as shown in FIG. 26;
(d) a Thermogravimetric Thermal Analysis (TGA) thermogram substantially the same as shown in FIG. 27; or
(e) combinations thereof.

In some embodiments of Compound 1 Sulfate salt, the crystalline form has an X-ray powder diffraction (XRPD) pattern substantially the same as shown in FIG. 25 as measured using Cu Kα.radiation.

In some embodiments of Compound 1 Sulfate salt, the crystalline form has an X-ray powder diffraction (XRPD) pattern with peaks found in Table 9 as measured using Cu Kα.radiation.

In some embodiments of Compound 1 Sulfate salt, the crystalline form has an X-ray powder diffraction (XRPD) pattern with peaks at 4.9±0.2° 2θ, 17.2±0.2° 2θ, and 21.5±0.2° 2θ as measured using Cu Kα.radiation.

In some embodiments of Compound 1 Sulfate salt, the X-ray powder diffraction (XRPD) pattern further comprises peaks at 14.2±0.2° 2θ, 17.8±0.2° 2θ, and 25.1±0.2° 2θ as measured using Cu Kα.radiation.

In some embodiments of Compound 1 Sulfate salt, the X-ray powder diffraction (XRPD) pattern further comprises peaks at 10.2±0.2° 2θ, 16.6±0.2° 2θ, and 28.1±0.2° 2θ as measured using Cu Kα.radiation.

In some embodiments of Compound 1 Sulfate salt, the crystalline form has an X-ray powder diffraction (XRPD) pattern with peaks at 4.9±0.2° 2θ, 10.2±0.2° 2θ, 14.2±0.2° 2θ, 16.6±0.2° 2θ, 17.2±0.2° 2θ, 17.8±0.2° 2θ, 21.5±0.2° 2θ, 25.1±0.2° 2θ, and 28.1±0.2° 2θ as measured using Cu Kα.radiation.

In some embodiments of Compound 1 Sulfate salt, the crystalline form has an X-ray powder diffraction (XRPD) pattern with at least two peaks selected from 4.9±0.2° 2θ, 10.2±0.2° 2θ, 14.2±0.2° 2θ, 16.6±0.2° 2θ, 17.2±0.2° 2θ, 17.8±0.2° 2θ, 21.5±0.2° 2θ, 25.1±0.2° 2θ, and 28.1±0.2° 2θ as measured using Cu Kα.radiation.

In some embodiments of Compound 1 Sulfate salt, the crystalline form has an X-ray powder diffraction (XRPD) pattern with at least three peaks selected from 4.9±0.2° 2θ, 10.2±0.2° 2θ, 14.2±0.2° 2θ, 16.6±0.2° 2θ, 17.2±0.2° 2θ, 17.8±0.2° 2θ, 21.5±0.2° 2θ, 25.1±0.2° 2θ, and 28.1±0.2° 2θ as measured using Cu Kα.radiation.

In some embodiments of Compound 1 Sulfate salt, the crystalline form has an X-ray powder diffraction (XRPD) pattern with at least four peaks selected from 4.9±0.2° 2θ, 10.2±0.2° 2θ, 14.2±0.2° 2θ, 16.6±0.2° 2θ, 17.2±0.2° 2θ, 17.8±0.2° 2θ, 21.5±0.2° 2θ, 25.1±0.2° 2θ, and 28.1±0.2° 2θ as measured using Cu Kα.radiation.

In some embodiments of Compound 1 Sulfate salt, the crystalline form has an X-ray powder diffraction (XRPD) pattern with at least five peaks selected from 4.9±0.2° 2θ, 10.2±0.2° 2θ, 14.2±0.2° 2θ, 16.6±0.2° 2θ, 17.2±0.2° 2θ, 17.8±0.2° 2θ, 21.5±0.2° 2θ, 25.1±0.2° 2θ, and 28.1±0.2° 2θ as measured using Cu Kα.radiation.

In some embodiments of Compound 1 Sulfate salt, the crystalline form has an X-ray powder diffraction (XRPD) pattern with at least six peaks selected from 4.9±0.2° 2θ, 10.2±0.2° 2θ, 14.2±0.2° 2θ, 16.6±0.2° 2θ, 17.2±0.2° 2θ, 17.8±0.2° 2θ, 21.5±0.2° 2θ, 25.1±0.2° 2θ, and 28.1±0.2° 2θ as measured using Cu Kα.radiation.

In some embodiments of Compound 1 Sulfate salt, the crystalline form has an X-ray powder diffraction (XRPD) pattern with at least seven peaks selected from 4.9±0.2° 2θ, 10.2±0.2° 2θ, 14.2±0.2° 2θ, 16.6±0.2° 2θ, 17.2±0.2° 2θ, 17.8±0.2° 2θ, 21.5±0.2° 2θ, 25.1±0.2° 2θ, and 28.1±0.2° 2θ as measured using Cu Kα.radiation.

In some embodiments of Compound 1 Sulfate salt, the crystalline form has an X-ray powder diffraction (XRPD) pattern with at least eight peaks selected from 4.9±0.2° 2θ, 10.2±0.2° 2θ, 14.2±0.2° 2θ, 16.6±0.2° 2θ, 17.2±0.2° 2θ, 17.8±0.2° 2θ, 21.5±0.2° 2θ, 25.1±0.2° 2θ, and 28.1±0.2° 2θ as measured using Cu Kα.radiation.

In some embodiments of Compound 1 Sulfate salt, the Differential Scanning Calorimetry (DSC) thermogram is substantially the same as shown in FIG. 26.

In some embodiments of Compound 1 Sulfate salt, the Thermogravimetric Thermal Analysis (TGA) thermogram is substantially the same as shown in FIG. 27.

In some embodiments of Compound 1 Sulfate salt, the crystalline form is an anhydrate.

TABLE 9
XRPD peaks table of Compound 1 Sulfate salt Type A

| | Diffraction angle | Rel. Int. |
|---|---|---|
| Peak# | ( [symbol] , 2 θ) | (%) |
| 1 | 4.9 | 100.00% |
| 2 | 6.6 | 13.80% |
| 3 | 9.8 | 7.00% |
| 4 | 10.2 | 18.30% |
| 5 | 12.3 | 5.40% |
| 6 | 13.2 | 5.20% |
| 7 | 14.2 | 69.60% |
| 8 | 14.5 | 38.80% |
| 9 | 16.6 | 27.60% |
| 10 | 17.2 | 59.90% |
| 11 | 17.8 | 87.70% |
| 12 | 18.4 | 10.30% |
| 13 | 20.4 | 15.10% |
| 14 | 21.5 | 44.10% |
| 15 | 22.1 | 15.20% |
| 16 | 23.2 | 15.00% |
| 17 | 24.0 | 6.00% |
| 18 | 24.5 | 13.80% |
| 19 | 25.1 | 39.90% |
| 20 | 25.5 | 35.10% |
| 21 | 26.2 | 13.70% |
| 22 | 26.7 | 9.50% |
| 23 | 27.6 | 25.00% |
| 24 | 28.1 | 46.00% |
| 25 | 29.0 | 16.60% |
| 26 | 31.9 | 3.40% |
| 27 | 33.0 | 10.00% |
| 28 | 37.4 | 4.90% |

### Compound 1 Sulfate Salt Type B

Disclosed herein is Compound 1 Sulfate salt Type B. In some embodiments, the crystalline form is Compound 1 Sulfate salt characterized as having at least one of the following properties:
(a) an X-Ray powder diffraction (XRPD) pattern substantially the same as shown in FIG. 28 as measured using Cu Kα.radiation;
(b) an X-Ray powder diffraction (XRPD) pattern with peaks at 5.5±0.2° 2θ, 17.6±0.2° 2θ, and 20.3±0.2° 2θ as measured using Cu Kα.radiation;
(c) a Differential Scanning Calorimetry (DSC) thermogram substantially the same as shown in FIG. 29;
(d) a Thermogravimetric Thermal Analysis (TGA) thermogram substantially the same as shown in FIG. 30; or
(e) combinations thereof.

In some embodiments of Compound 1 Sulfate salt, the crystalline form has an X-ray powder diffraction (XRPD) pattern substantially the same as shown in FIG. 28 as measured using Cu Kα.radiation.

In some embodiments of Compound 1 Sulfate salt, the crystalline form has an X-ray powder diffraction (XRPD) pattern with peaks found in Table 10 as measured using Cu Kα.radiation.

In some embodiments of Compound 1 Sulfate salt, the crystalline form has an X-ray powder diffraction (XRPD) pattern with peaks at 5.5±0.2° 2θ, 17.6±0.2° 2θ, and 20.3±0.2° 2θ as measured using Cu Kα.radiation.

In some embodiments of Compound 1 Sulfate salt, the X-ray powder diffraction (XRPD) pattern further comprises peaks at 16.5±0.2° 2θ, 18.3±0.2° 2θ, and 24.9±0.2° 2θ as measured using Cu Kα.radiation.

In some embodiments of Compound 1 Sulfate salt, the X-ray powder diffraction (XRPD) pattern further comprises peaks at 19.6±0.2° 2θ, 21.0±0.2° 2θ, and 22.1±0.2° 2θ as measured using Cu Kα.radiation.

In some embodiments of Compound 1 Sulfate salt, the crystalline form has an X-ray powder diffraction (XRPD) pattern with peaks at 5.5±0.2° 2θ, 16.5±0.2° 2θ, 17.6±0.2° 2θ, 18.3±0.2° 2θ, 19.6±0.2° 2θ, 20.3±0.2° 2θ, 21.0±0.2° 2θ, 22.1±0.2° 2θ, and 24.9±0.2° 2θ as measured using Cu Kα.radiation.

In some embodiments of Compound 1 Sulfate salt, the crystalline form has an X-ray powder diffraction (XRPD) pattern with at least two peaks selected from 5.5±0.2° 2θ, 16.5±0.2° 2θ, 17.6±0.2° 2θ, 18.3±0.2° 2θ, 19.6±0.2° 2θ, 20.3±0.2° 2θ, 21.0±0.2° 2θ, 22.1±0.2° 2θ, and 24.9±0.2° 2θ, and 28.1±0.2° 2θ as measured using Cu Kα.radiation.

In some embodiments of Compound 1 Sulfate salt, the crystalline form has an X-ray powder diffraction (XRPD) pattern with at least three peaks selected from 5.5±0.2° 2θ, 16.5±0.2° 2θ, 17.6±0.2° 2θ, 18.3±0.2° 2θ, 19.6±0.2° 2θ, 20.3±0.2° 2θ, 21.0±0.2° 2θ, 22.1±0.2° 2θ, and 24.9±0.2° 2θ, and 28.1±0.2° 2θ as measured using Cu Kα.radiation.

In some embodiments of Compound 1 Sulfate salt, the crystalline form has an X-ray powder diffraction (XRPD) pattern with at least four peaks selected from 5.5±0.2° 2θ, 16.5±0.2° 2θ, 17.6±0.2° 2θ, 18.3±0.2° 2θ, 19.6±0.2° 2θ, 20.3±0.2° 2θ, 21.0±0.2° 2θ, 22.1±0.2° 2θ, and 24.9±0.2° 2θ, and 28.1±0.2° 2θ as measured using Cu Kα.radiation.

In some embodiments of Compound 1 Sulfate salt, the crystalline form has an X-ray powder diffraction (XRPD) pattern with at least five peaks selected from 5.5±0.2° 2θ, 16.5±0.2° 2θ, 17.6±0.2° 2θ, 18.3±0.2° 2θ, 19.6±0.2° 2θ, 20.3±0.2° 2θ, 21.0±0.2° 2θ, 22.1±0.2° 2θ, and 24.9±0.2° 2θ, and 28.1±0.2° 2θ as measured using Cu Kα.radiation.

In some embodiments of Compound 1 Sulfate salt, the crystalline form has an X-ray powder diffraction (XRPD) pattern with at least six peaks selected from 5.5±0.2° 2θ, 16.5±0.2° 2θ, 17.6±0.2° 2θ, 18.3±0.2° 2θ, 19.6±0.2° 2θ, 20.3±0.2° 2θ, 21.0±0.2° 2θ, 22.1±0.2° 2θ, and 24.9±0.2° 2θ as measured using Cu Kα.radiation.

In some embodiments of Compound 1 Sulfate salt, the crystalline form has an X-ray powder diffraction (XRPD) pattern with at least seven peaks selected from 5.5±0.2° 2θ, 16.5±0.2° 2θ, 17.6±0.2° 2θ, 18.3±0.2° 2θ, 19.6±0.2° 2θ, 20.3±0.2° 2θ, 21.0±0.2° 2θ, 22.1±0.2° 2θ, and 24.9±0.2° 2θ as measured using Cu Kα.radiation.

In some embodiments of Compound 1 Sulfate salt, the crystalline form has an X-ray powder diffraction (XRPD) pattern with at least eight peaks selected from 5.5±0.2° 2θ, 16.5±0.2° 2θ, 17.6±0.2° 2θ, 18.3±0.2° 2θ, 19.6±0.2° 2θ, 20.3±0.2° 2θ, 21.0±0.2° 2θ, 22.1±0.2° 2θ, and 24.9±0.2° 2θ as measured using Cu Kα.radiation.

In some embodiments of Compound 1 Sulfate salt, the Differential Scanning Calorimetry (DSC) thermogram is substantially the same as shown in FIG. 29.

In some embodiments of Compound 1 Sulfate salt, the Thermogravimetric Thermal Analysis (TGA) thermogram is substantially the same as shown in FIG. 30.

In some embodiments of Compound 1 Sulfate salt, the crystalline form is an anhydrate.

TABLE 10
XRPD peaks table of Compound 1 sulfate salt Type B

| | Diffraction angle | Rel. Int. |
|---|---|---|
| Peak# | ( [symbol] , 2 θ) | (%) |
| 1 | 5.5 | 100.00% |
| 2 | 11.5 | 1.90% |
| 3 | 14.0 | 2.70% |
| 4 | 14.7 | 2.00% |
| 5 | 15.7 | 5.90% |
| 6 | 16.5 | 58.90% |
| 7 | 17.6 | 17.90% |
| 8 | 18.3 | 20.50% |
| 9 | 18.5 | 9.20% |
| 10 | 19.0 | 5.60% |
| 11 | 19.6 | 8.90% |
| 12 | 20.3 | 14.40% |
| 13 | 20.5 | 6.70% |
| 14 | 21.0 | 8.90% |
| 15 | 21.5 | 2.80% |
| 16 | 22.1 | 10.10% |
| 17 | 22.5 | 7.30% |
| 18 | 23.9 | 5.30% |
| 19 | 24.9 | 17.00% |
| 20 | 27.1 | 3.30% |
| 21 | 28.2 | 3.00% |
| 22 | 28.3 | 8.10% |
| 23 | 30.5 | 3.50% |
| 24 | 31.2 | 3.40% |
| 25 | 31.6 | 1.80% |

### Compound 1 Methanesulfonate Salt Type A

Disclosed herein is Compound 1 Methanesulfonate salt Type A. In some embodiments, the crystalline form is Compound 1 Methanesulfonate salt characterized as having at least one of the following properties:
(a) an X-Ray powder diffraction (XRPD) pattern substantially the same as shown in FIG. 31 as measured using Cu Kα.radiation;
(b) an X-Ray powder diffraction (XRPD) pattern with peaks at 5.2±0.2° 2θ, 17.8±0.2° 2θ, and 20.5±0.2° 2θ as measured using Cu Kα.radiation;
(c) a Differential Scanning Calorimetry (DSC) thermogram substantially the same as shown in FIG. 32;
(d) a Thermogravimetric Thermal Analysis (TGA) thermogram substantially the same as shown in FIG. 33; or
(e) combinations thereof.

In some embodiments of Compound 1 Methanesulfonate salt, the crystalline form has an X-ray powder diffraction (XRPD) pattern substantially the same as shown in FIG. 31 as measured using Cu Kα.radiation.

In some embodiments of Compound 1 Methanesulfonate salt, the crystalline form has an X-ray powder diffraction (XRPD) pattern with peaks found in Table 11 as measured using Cu Kα.radiation.

In some embodiments of Compound 1 Methanesulfonate salt, the crystalline form has an X-ray powder diffraction (XRPD) pattern with peaks at 5.2±0.2° 2θ, 17.8±0.2° 2θ, and 20.5±0.2° 2θ as measured using Cu Kα.radiation.

In some embodiments of Compound 1 Methanesulfonate salt, the X-ray powder diffraction (XRPD) pattern further comprises peaks at 14.1±0.2° 2θ, 18.8±0.2° 2θ, and 26.0±0.2° 2θ as measured using Cu Kα.radiation.

In some embodiments of Compound 1 Methanesulfonate salt, the X-ray powder diffraction (XRPD) pattern further comprises peaks at 15.2±0.2° 2θ and 25.0±0.2° 2θ as measured using Cu Kα.radiation.

In some embodiments of Compound 1 Methanesulfonate salt, the crystalline form has an X-ray powder diffraction (XRPD) pattern with peaks at 5.2±0.2° 2θ, 14.1±0.2° 2θ, 15.2±0.2° 2θ, 17.8±0.2° 2θ, 18.8±0.2° 2θ, 20.5±0.2° 2θ, 25.0±0.2° 2θ, and 26.0±0.2° 2θ as measured using Cu Kα.radiation.

In some embodiments of Compound 1 Methanesulfonate salt, the crystalline form has an X-ray powder diffraction (XRPD) pattern with at least two peaks selected from 5.2±0.2° 2θ, 14.1±0.2° 2θ, 15.2±0.2° 2θ, 17.8±0.2° 2θ, 18.8±0.2° 2θ, 20.5±0.2° 2θ, 25.0±0.2° 2θ, and 26.0±0.2° 2θ as measured using Cu Kα.radiation.

In some embodiments of Compound 1 Methanesulfonate salt, the crystalline form has an X-ray powder diffraction (XRPD) pattern with at least three peaks selected from 5.2±0.2° 2θ, 14.1±0.2° 2θ, 15.2±0.2° 2θ, 17.8±0.2° 2θ, 18.8±0.2° 2θ, 20.5±0.2° 2θ, 25.0±0.2° 2θ, and 26.0±0.2° 2θ as measured using Cu Kα.radiation.

In some embodiments of Compound 1 Methanesulfonate salt, the crystalline form has an X-ray powder diffraction (XRPD) pattern with at least four peaks selected from 5.2±0.2° 2θ, 14.1±0.2° 2θ, 15.2±0.2° 2θ, 17.8±0.2° 2θ, 18.8±0.2° 2θ, 20.5±0.2° 2θ, 25.0±0.2° 2θ, and 26.0±0.2° 2θ as measured using Cu Kα.radiation.

In some embodiments of Compound 1 Methanesulfonate salt, the crystalline form has an X-ray powder diffraction (XRPD) pattern with at least five peaks selected from 5.2±0.2° 2θ, 14.1±0.2° 2θ, 15.2±0.2° 2θ, 17.8±0.2° 2θ, 18.8±0.2° 2θ, 20.5±0.2° 2θ, 25.0±0.2° 2θ, and 26.0±0.2° 2θ as measured using Cu Kα.radiation.

In some embodiments of Compound 1 Methanesulfonate salt, the crystalline form has an X-ray powder diffraction (XRPD) pattern with at least six peaks selected from 5.2±0.2° 2θ, 14.1±0.2° 2θ, 15.2±0.2° 2θ, 17.8±0.2° 2θ, 18.8±0.2° 2θ, 20.5±0.2° 2θ, 25.0±0.2° 2θ, and 26.0±0.2° 2θ as measured using Cu Kα.radiation.

In some embodiments of Compound 1 Methanesulfonate salt, the crystalline form has an X-ray powder diffraction (XRPD) pattern with at least seven peaks selected from 5.2±0.2° 2θ, 14.1±0.2° 2θ, 15.2±0.2° 2θ, 17.8±0.2° 2θ, 18.8±0.2° 2θ, 20.5±0.2° 2θ, 25.0±0.2° 2θ, and 26.0±0.2° 2θ as measured using Cu Kα.radiation.

In some embodiments of Compound 1 Methanesulfonate salt, the crystalline form has an X-ray powder diffraction (XRPD) pattern with at least eight peaks selected from 5.2±0.2° 2θ, 14.1±0.2° 2θ, 15.2±0.2° 2θ, 17.8±0.2° 2θ, 18.8±0.2° 2θ, 20.5±0.2° 2θ, 25.0±0.2° 2θ, and 26.0±0.2° 2θ as measured using Cu Kα.radiation.

In some embodiments of Compound 1 Methanesulfonate salt, the Differential Scanning Calorimetry (DSC) thermogram is substantially the same as shown in FIG. 32.

In some embodiments of Compound 1 Methanesulfonate salt, the Thermogravimetric Thermal Analysis (TGA) thermogram is substantially the same as shown in FIG. 33.

In some embodiments of Compound 1 Methanesulfonate salt, the crystalline form is an anhydrate.

TABLE 11
XRPD peaks table of Compound 1
Methanesulfonate salt Type A

| | Diffraction angle | Rel. Int. |
|---|---|---|
| Peak# | ( [symbol] , 2 θ) | (%) |
| 1 | 5.2 | 100.00% |
| 2 | 10.4 | 2.70% |
| 3 | 11.7 | 2.60% |
| 4 | 13.0 | 3.10% |
| 5 | 14.1 | 29.00% |
| 6 | 14.3 | 17.10% |
| 7 | 15.2 | 8.90% |
| 8 | 16.3 | 4.90% |
| 9 | 17.8 | 26.70% |
| 10 | 18.4 | 6.80% |
| 11 | 18.8 | 16.70% |
| 12 | 19.9 | 15.20% |
| 13 | 20.1 | 22.50% |
| 14 | 20.5 | 37.30% |
| 15 | 21.5 | 5.70% |
| 16 | 22.2 | 7.10% |
| 17 | 23.9 | 8.20% |
| 18 | 25.0 | 13.40% |
| 19 | 26.0 | 24.90% |
| 20 | 28.9 | 9.40% |
| 21 | 30.4 | 3.10% |
| 22 | 32.2 | 4.00% |

### Compound 1 Benzenesulfonate Salt Type A

Disclosed herein is Compound 1 Benzenesulfonate salt Type A. In some embodiments, the crystalline form is Compound 1 Benzenesulfonate salt characterized as having at least one of the following properties:
(a) an X-Ray powder diffraction (XRPD) pattern substantially the same as shown in FIG. 34 as measured using Cu Kα.radiation;
(b) an X-Ray powder diffraction (XRPD) pattern with peaks at 4.4±0.2° 2θ, 17.0±0.2° 2θ, and 20.4±0.2° 2θ as measured using Cu Kα.radiation;
(c) a Differential Scanning Calorimetry (DSC) thermogram substantially the same as shown in FIG. 35;
(d) a Thermogravimetric Thermal Analysis (TGA) thermogram substantially the same as shown in FIG. 36; or
(e) combinations thereof.

In some embodiments of Compound 1 Benzenesulfonate salt, the crystalline form has an X-ray powder diffraction (XRPD) pattern substantially the same as shown in FIG. 34 as measured using Cu Kα.radiation.

In some embodiments of Compound 1 Benzenesulfonate salt, the crystalline form has an X-ray powder diffraction (XRPD) pattern with peaks found in Table 12 as measured using Cu Kα.radiation.

In some embodiments of Compound 1 Benzenesulfonate salt, the crystalline form has an X-ray powder diffraction (XRPD) pattern with peaks at 4.4±0.2° 2θ, 17.0±0.2° 2θ, and 20.4±0.2° 2θ as measured using Cu Kα.radiation.

In some embodiments of Compound 1 Benzenesulfonate salt, the X-ray powder diffraction (XRPD) pattern further comprises peaks at 14.4±0.2° 2θ and 19.3±0.2° 2θ as measured using Cu Kα.radiation.

In some embodiments of Compound 1 Benzenesulfonate salt, the X-ray powder diffraction (XRPD) pattern further comprises peaks at 8.9±0.2° 2θ, 13.2±0.2° 2θ, 15.6±0.2° 2θ, and 23.7±0.2° 2θ as measured using Cu Kα.radiation.

In some embodiments of Compound 1 Benzenesulfonate salt, the crystalline form has an X-ray powder diffraction (XRPD) pattern with peaks at 4.4±0.2° 2θ, 8.9±0.2° 2θ, 13.2±0.2° 2θ, 14.4±0.2° 2θ, 15.6±0.2° 2θ, 17.0±0.2° 2θ, 19.3±0.2° 2θ, 20.4±0.2° 2θ, and 23.7±0.2° 2θ as measured using Cu Kα.radiation.

In some embodiments of Compound 1 Benzenesulfonate salt, the crystalline form has an X-ray powder diffraction (XRPD) pattern with at least two peaks selected from 4.4±0.2° 2θ, 8.9±0.2° 2θ, 13.2±0.2° 2θ, 14.4±0.2° 2θ, 15.6±0.2° 2θ, 17.0±0.2° 2θ, 19.3±0.2° 2θ, 20.4±0.2° 2θ, and 23.7±0.2° 2θ as measured using Cu Kα.radiation.

In some embodiments of Compound 1 Benzenesulfonate salt, the crystalline form has an X-ray powder diffraction (XRPD) pattern with at least three peaks selected from 4.4±0.2° 2θ, 8.9±0.2° 2θ, 13.2±0.2° 2θ, 14.4±0.2° 2θ, 15.6±0.2° 2θ, 17.0±0.2° 2θ, 19.3±0.2° 2θ, 20.4±0.2° 2θ, and 23.7±0.2° 2θ as measured using Cu Kα.radiation.

In some embodiments of Compound 1 Benzenesulfonate salt, the crystalline form has an X-ray powder diffraction (XRPD) pattern with at least four peaks selected from 4.4±0.2° 2θ, 8.9±0.2° 2θ, 13.2±0.2° 2θ, 14.4±0.2° 2θ, 15.6±0.2° 2θ, 17.0±0.2° 2θ, 19.3±0.2° 2θ, 20.4±0.2° 2θ, and 23.7±0.2° 2θ as measured using Cu Kα.radiation.

In some embodiments of Compound 1 Benzenesulfonate salt, the crystalline form has an X-ray powder diffraction (XRPD) pattern with at least five peaks selected from 4.4±0.2° 2θ, 8.9±0.2° 2θ, 132±0.2° 2θ, 14.4±0.2° 2θ, 15.6±0.2° 2θ, 17.0±0.2° 2θ, 19.3±0.2° 2θ, 20.4±0.2° 2θ, and 23.7±0.2° 2θ as measured using Cu Kα.radiation.

In some embodiments of Compound 1 Benzenesulfonate salt, the crystalline form has an X-ray powder diffraction (XRPD) pattern with at least six peaks selected from 4.4±0.2° 2θ, 8.9±0.2° 2θ, 13.2±0.2° 2θ, 14.4±0.2° 2θ, 15.6±0.2° 2θ, 17.0±0.2° 2θ, 19.3±0.2° 2θ, 20.4±0.2° 2θ, and 23.7±0.2° 2θ as measured using Cu Kα.radiation.

In some embodiments of Compound 1 Benzenesulfonate salt, the crystalline form has an X-ray powder diffraction (XRPD) pattern with at least seven peaks selected from 4.4±0.2° 2θ, 8.9±0.2° 2θ, 13.2±0.2° 2θ, 14.4±0.2° 2θ, 15.6±0.2° 2θ, 17.0±0.2° 2θ, 19.3±0.2° 2θ, 20.4±0.2° 2θ, and 23.7±0.2° 2θ as measured using Cu Kα.radiation.

In some embodiments of Compound 1 Benzenesulfonate salt, the crystalline form has an X-ray powder diffraction (XRPD) pattern with at least eight peaks selected from 4.4±0.2° 2θ, 8.9±0.2° 2θ, 13.2±0.2° 2θ, 14.4±0.2° 2θ, 15.6±0.2° 2θ, 17.0±0.2° 2θ, 19.3±0.2° 2θ, 20.4±0.2° 2θ, and 23.7±0.2° 2θ as measured using Cu Kα.radiation.

In some embodiments of Compound 1 Benzenesulfonate salt, the Differential Scanning Calorimetry (DSC) thermogram is substantially the same as shown in FIG. 35.

In some embodiments of Compound 1 Benzenesulfonate salt, the Thermogravimetric Thermal Analysis (TGA) thermogram is substantially the same as shown in FIG. 36.

In some embodiments of Compound 1 Benzenesulfonate salt, the crystalline form is an anhydrate.

TABLE 12
XRPD peaks table of Compound 1
Benzenesulfonate salt Type A

| | Diffraction angle | Rel. Int. |
|---|---|---|
| Peak# | ( [symbol] , 2 θ) | (%) |
| 1 | 4.4 | 100.00% |
| 2 | 8.9 | 16.30% |
| 3 | 11.8 | 6.70% |
| 4 | 13.2 | 18.20% |
| 5 | 13.5 | 3.90% |
| 6 | 14.4 | 22.00% |
| 7 | 15.3 | 11.00% |
| 8 | 15.6 | 13.80% |
| 9 | 16.3 | 6.70% |
| 10 | 17.0 | 29.80% |
| 11 | 19.3 | 24.30% |
| 12 | 20.4 | 38.40% |
| 13 | 21.1 | 1.90% |
| 14 | 21.7 | 5.50% |
| 15 | 22.4 | 4.30% |
| 16 | 23.7 | 27.40% |
| 17 | 24.9 | 8.90% |
| 18 | 25.8 | 9.10% |
| 19 | 26.9 | 7.00% |
| 20 | 27.5 | 12.40% |
| 21 | 31.0 | 5.00% |
| 22 | 31.5 | 2.80% |
| 23 | 32.1 | 7.20% |
| 24 | 32.6 | 4.50% |

### Compound 1 Naphthalene-1,5-Disulfonate Salt Type A

Disclosed herein is Compound 1 Naphthalene-1,5-disulfonate salt Type A. In some embodiments, the crystalline form is Compound 1 Naphthalene-1,5-disulfonate salt characterized as having at least one of the following properties:
(a) an X-Ray powder diffraction (XRPD) pattern substantially the same as shown in FIG. 37 as measured using Cu Kα.radiation;
(b) an X-Ray powder diffraction (XRPD) pattern with peaks at 4.3±0.2° 2θ, 14.8±0.2° 2θ, and 19.2±0.2° 2θ as measured using Cu Kα.radiation;
(c) a Differential Scanning Calorimetry (DSC) thermogram substantially the same as shown in FIG. 38;
(d) a Thermogravimetric Thermal Analysis (TGA) thermogram substantially the same as shown in FIG. 39; or
(e) combinations thereof.

In some embodiments of Compound 1 Naphthalene-1,5-disulfonate salt, the crystalline form has an X-ray powder diffraction (XRPD) pattern substantially the same as shown in FIG. 37 as measured using Cu Kα.radiation.

In some embodiments of Compound 1 Naphthalene-1,5-disulfonate salt, the crystalline form has an X-ray powder diffraction (XRPD) pattern with peaks found in Table 13 as measured using Cu Kα.radiation.

In some embodiments of Compound 1 Naphthalene-1,5-disulfonate salt, the crystalline form has an X-ray powder diffraction (XRPD) pattern with peaks at 4.3±0.2° 2θ, 14.8±0.2° 2θ, and 19.2±0.2° 2θ as measured using Cu Kα.radiation.

In some embodiments of Compound 1 Naphthalene-1,5-disulfonate salt, the X-ray powder diffraction (XRPD) pattern further comprises peaks at 12.0±0.2° 2θ, 17.2±0.2° 2θ, and 25.6±0.2° 2θ as measured using Cu Kα.radiation.

In some embodiments of Compound 1 Naphthalene-1,5-disulfonate salt, the X-ray powder diffraction (XRPD) pattern further comprises peaks at 13.9±0.2° 2θ, 20.4±0.2° 2θ, and 25.8±0.2° 2θ as measured using Cu Kα.radiation.

In some embodiments of Compound 1 Naphthalene-1,5-disulfonate salt, the crystalline form has an X-ray powder diffraction (XRPD) pattern with peaks at 4.3±0.2° 2θ, 12.0±0.2° 2θ, 13.9±0.2° 2θ, 14.8±0.2° 2θ, 17.2±0.2° 2θ, 19.2±0.2° 2θ, 20.4±0.2° 2θ, 25.6±0.2° 2θ, and 25.8±0.2° 2θ as measured using Cu Kα.radiation.

In some embodiments of Compound 1 Naphthalene-1,5-disulfonate salt, the crystalline form has an X-ray powder diffraction (XRPD) pattern with at least two peaks selected from 4.3±0.2° 2θ, 12.0±0.2° 2θ, 13.9±0.2° 2θ, 14.8±0.2° 2θ, 17.2±0.2° 2θ, 19.2±0.2° 2θ, 20.4±0.2° 2θ, 25.6±0.2° 2θ, and 25.8±0.2° 2θ as measured using Cu Kα.radiation.

In some embodiments of Compound 1 Naphthalene-1,5-disulfonate salt, the crystalline form has an X-ray powder diffraction (XRPD) pattern with at least three peaks selected from 4.3±0.2° 2θ, 12.0±0.2° 2θ, 13.9±0.2° 2θ, 14.8±0.2° 2θ, 17.2±0.2° 2θ, 19.2±0.2° 2θ, 20.4±0.2° 2θ, 25.6±0.2° 2θ, and 25.8±0.2° 2θ as measured using Cu Kα.radiation.

In some embodiments of Compound 1 Naphthalene-1,5-disulfonate salt, the crystalline form has an X-ray powder diffraction (XRPD) pattern with at least four peaks selected from 4.3±0.2° 2θ, 12.0±0.2° 2θ, 13.9±0.2° 2θ, 14.8±0.2° 2θ, 17.2±0.2° 2θ, 19.2±0.2° 2θ, 20.4±0.2° 2θ, 25.6±0.2° 2θ, and 25.8±0.2° 2θ as measured using Cu Kα.radiation.

In some embodiments of Compound 1 Naphthalene-1,5-disulfonate salt, the crystalline form has an X-ray powder diffraction (XRPD) pattern with at least five peaks selected from 4.3±0.2° 2θ, 12.0±0.2° 2θ, 13.9±0.2° 2θ, 14.8±0.2° 2θ, 17.2±0.2° 2θ, 19.2±0.2° 2θ, 20.4±0.2° 2θ, 25.6±0.2° 2θ, and 25.8±0.2° 2θ as measured using Cu Kα.radiation.

In some embodiments of Compound 1 Naphthalene-1,5-disulfonate salt, the crystalline form has an X-ray powder diffraction (XRPD) pattern with at least six peaks selected from 4.3±0.2° 2θ, 12.0±0.2° 2θ, 13.9±0.2° 2θ, 14.8±0.2° 2θ, 17.2±0.2° 2θ, 19.2±0.2° 2θ, 20.4±0.2° 2θ, 25.6±0.2° 2θ, and 25.8±0.2° 2θ as measured using Cu Kα.radiation.

In some embodiments of Compound 1 Naphthalene-1,5-disulfonate salt, the crystalline form has an X-ray powder diffraction (XRPD) pattern with at least seven peaks selected from 4.3±0.2° 2θ, 12.0±0.2° 2θ, 13.9±0.2° 2θ, 14.8±0.2° 2θ, 17.2±0.2° 2θ, 19.2±0.2° 2θ, 20.4±0.2° 2θ, 25.6±0.2° 2θ, and 25.8±0.2° 2θ as measured using Cu Kα.radiation.

In some embodiments of Compound 1 Naphthalene-1,5-disulfonate salt, the crystalline form has an X-ray powder diffraction (XRPD) pattern with at least eight peaks selected from 4.3±0.2° 2θ, 12.0±0.2° 2θ, 13.9±0.2° 2θ, 14.8±0.2° 2θ, 17.2±0.2° 2θ, 19.2±0.2° 2θ, 20.4±0.2° 2θ, 25.6±0.2° 2θ, and 25.8±0.2° 2θ as measured using Cu Kα.radiation.

In some embodiments of Compound 1 Naphthalene-1,5-disulfonate salt, the Differential Scanning Calorimetry (DSC) thermogram is substantially the same as shown in FIG. 38.

In some embodiments of Compound 1 Naphthalene-1,5-disulfonate salt, the Thermogravimetric Thermal Analysis (TGA) thermogram is substantially the same as shown in FIG. 39.

In some embodiments of Compound 1 Naphthalene-1,5-disulfonate salt, the crystalline form is a hydrate.

TABLE 13
XRPD peaks table of Compound 1 Naphthalene-
1,5-disulfonate salt Type A

| | Diffraction angle | Rel. Int. |
|---|---|---|
| Peak# | ( [symbol] , 2 θ) | (%) |
| 1 | 4.3 | 100.00% |
| 2 | 7.0 | 3.70% |
| 3 | 8.0 | 3.10% |
| 4 | 8.4 | 3.40% |
| 5 | 9.3 | 7.20% |
| 6 | 10.1 | 7.30% |
| 7 | 11.1 | 4.60% |
| 8 | 12.0 | 15.40% |
| 9 | 12.8 | 10.30% |
| 10 | 13.9 | 13.20% |
| 11 | 14.8 | 21.50% |
| 12 | 15.6 | 7.40% |
| 13 | 16.5 | 5.00% |
| 14 | 17.0 | 8.30% |
| 15 | 17.2 | 15.40% |
| 16 | 17.7 | 7.30% |
| 17 | 18.7 | 7.90% |
| 18 | 19.2 | 30.70% |
| 19 | 19.5 | 8.30% |
| 20 | 20.2 | 7.20% |
| 21 | 20.4 | 8.90% |
| 22 | 21.3 | 10.20% |
| 23 | 21.8 | 3.80% |
| 24 | 22.4 | 4.60% |
| 25 | 23.6 | 4.70% |
| 26 | 24.5 | 14.20% |
| 27 | 25.6 | 23.00% |
| 28 | 25.8 | 44.30% |
| 29 | 26.0 | 18.30% |
| 30 | 27.7 | 12.10% |
| 31 | 29.7 | 4.50% |
| 32 | 30.2 | 4.90% |
| 33 | 31.5 | 5.90% |

### Compound 1 Naphthalene-1,5-Disulfonate Salt Type B

Disclosed herein is Compound 1 Naphthalene-1,5-disulfonate salt Type B. In some embodiments, the crystalline form is Compound 1 Naphthalene-1,5-disulfonate salt characterized as having at least one of the following properties:
(a) an X-Ray powder diffraction (XRPD) pattern substantially the same as shown in FIG. 40 as measured using Cu Kα.radiation;
(b) an X-Ray powder diffraction (XRPD) pattern with peaks at 5.2±0.2° 2θ, 16.1±0.2° 2θ, and 19.3±0.2° 2θ as measured using Cu Kα.radiation;
(c) a Differential Scanning Calorimetry (DSC) thermogram substantially the same as shown in FIG. 41;
(d) a Thermogravimetric Thermal Analysis (TGA) thermogram substantially the same as shown in FIG. 42; or
(e) combinations thereof.

In some embodiments of Compound 1 Naphthalene-1,5-disulfonate salt, the crystalline form has an X-ray powder diffraction (XRPD) pattern substantially the same as shown in FIG. 40 as measured using Cu Kα.radiation.

In some embodiments of Compound 1 Naphthalene-1,5-disulfonate salt, the crystalline form has an X-ray powder diffraction (XRPD) pattern with peaks found in Table 14 as measured using Cu Kα.radiation.

In some embodiments of Compound 1 Naphthalene-1,5-disulfonate salt, the crystalline form has an X-ray powder diffraction (XRPD) pattern with peaks at 5.2±0.2° 2θ, 16.1±0.2° 2θ, and 19.3±0.2° 2θ as measured using Cu Kα.radiation.

In some embodiments of Compound 1 Naphthalene-1,5-disulfonate salt, the X-ray powder diffraction (XRPD) pattern further comprises peaks at 12.7±0.2° 2θ, 18.7±0.2° 2θ, and 19.9±0.2° 2θ as measured using Cu Kα.radiation.

In some embodiments of Compound 1 Naphthalene-1,5-disulfonate salt, the X-ray powder diffraction (XRPD) pattern further comprises peaks at 22.7±0.2° 2θ, 24.6±0.2° 2θ, and 25.2±0.2° 2θ as measured using Cu Kα.radiation.

In some embodiments of Compound 1 Naphthalene-1,5-disulfonate salt, the crystalline form has an X-ray powder diffraction (XRPD) pattern with peaks at 5.2±0.2° 2θ, 12.7±0.2° 2θ, 16.1±0.2° 2θ, 18.7±0.2° 2θ, 19.3±0.2° 2θ, 19.9±0.2° 2θ, 22.7±0.2° 2θ, 24.6±0.2° 2θ, and 25.2±0.2° 2θ as measured using Cu Kα.radiation.

In some embodiments of Compound 1 Naphthalene-1,5-disulfonate salt, the crystalline form has an X-ray powder diffraction (XRPD) pattern with at least two peaks selected from 5.2±0.2° 2θ, 12.7±0.2° 2θ, 16.1±0.2° 2θ, 18.7±0.2° 2θ, 19.3±0.2° 2θ, 19.9±0.2° 2θ, 22.7±0.2° 2θ, 24.6±0.2° 2θ, and 25.2±0.2° 2θ as measured using Cu Kα.radiation.

In some embodiments of Compound 1 Naphthalene-1,5-disulfonate salt, the crystalline form has an X-ray powder diffraction (XRPD) pattern with at least three peaks selected from 5.2±0.2° 2θ, 12.7±0.2° 2θ, 16.1±0.2° 2θ, 18.7±0.2° 2θ, 19.3±0.2° 2θ, 19.9±0.2° 2θ, 22.7±0.2° 2θ, 24.6±0.2° 2θ, and 25.2±0.2° 2θ as measured using Cu Kα.radiation.

In some embodiments of Compound 1 Naphthalene-1,5-disulfonate salt, the crystalline form has an X-ray powder diffraction (XRPD) pattern with at least four peaks selected from 5.2±0.2° 2θ, 12.7±0.2° 2θ, 16.1±0.2° 2θ, 18.7±0.2° 2θ, 19.3±0.2° 2θ, 19.9±0.2° 2θ, 22.7±0.2° 2θ, 24.6±0.2° 2θ, and 25.2±0.2° 2θ as measured using Cu Kα.radiation.

In some embodiments of Compound 1 Naphthalene-1,5-disulfonate salt, the crystalline form has an X-ray powder diffraction (XRPD) pattern with at least five peaks selected from 5.2±0.2° 2θ, 12.7±0.2° 2θ, 16.1±0.2° 2θ, 18.7±0.2° 2θ, 19.3±0.2° 2θ, 19.9±0.2° 2θ, 22.7±0.2° 2θ, 24.6±0.2° 2θ, and 25.2±0.2° 2θ as measured using Cu Kα.radiation.

In some embodiments of Compound 1 Naphthalene-1,5-disulfonate salt, the crystalline form has an X-ray powder diffraction (XRPD) pattern with at least six peaks selected from 5.2±0.2° 2θ, 12.7±0.2° 2θ, 16.1±0.2° 2θ, 18.7±0.2° 2θ, 19.3±0.2° 2θ, 19.9±0.2° 2θ, 22.7±0.2° 2θ, 24.6±0.2° 2θ, and 25.2±0.2° 2θ as measured using Cu Kα.radiation.

In some embodiments of Compound 1 Naphthalene-1,5-disulfonate salt, the crystalline form has an X-ray powder diffraction (XRPD) pattern with at least seven peaks selected from 5.2±0.2° 2θ, 12.7±0.2° 2θ, 16.1±0.2° 2θ, 18.7±0.2° 2θ, 19.3±0.2° 2θ, 19.9±0.2° 2θ, 22.7±0.2° 2θ, 24.6±0.2° 2θ, and 25.2±0.2° 2θ as measured using Cu Kα.radiation.

In some embodiments of Compound 1 Naphthalene-1,5-disulfonate salt, the crystalline form has an X-ray powder diffraction (XRPD) pattern with at least eight peaks selected from 5.2±0.2° 2θ, 12.7±0.2° 2θ, 16.1±0.2° 2θ, 18.7±0.2° 2θ, 19.3±0.2° 2θ, 19.9±0.2° 2θ, 22.7±0.2° 2θ, 24.6±0.2° 2θ, and 25.2±0.2° 2θ as measured using Cu Kα.radiation.

In some embodiments of Compound 1 Naphthalene-1,5-disulfonate salt, the Differential Scanning Calorimetry (DSC) thermogram is substantially the same as shown in FIG. 41.

In some embodiments of Compound 1 Naphthalene-1,5-disulfonate salt, the Thermogravimetric Thermal Analysis (TGA) thermogram is substantially the same as shown in FIG. 42.

In some embodiments of Compound 1 Naphthalene-1,5-disulfonate salt, the crystalline form is a hydrate.

TABLE 14
XRPD peaks table of Compound 1 Naphthalene-
1,5-disulfonate salt Type B

| | Diffraction angle | Rel. Int. |
|---|---|---|
| Peak# | ( [symbol] , 2 θ) | (%) |
| 1 | 5.2 | 100.00% |
| 2 | 9.4 | 2.00% |
| 3 | 12.7 | 23.60% |
| 4 | 15.0 | 14.60% |
| 5 | 15.2 | 10.30% |
| 6 | 16.1 | 32.70% |
| 7 | 18.7 | 26.00% |
| 8 | 19.3 | 19.50% |
| 9 | 19.9 | 37.40% |
| 10 | 20.3 | 22.60% |
| 11 | 21.8 | 5.20% |
| 12 | 22.4 | 12.40% |
| 13 | 22.7 | 26.90% |
| 14 | 23.5 | 6.20% |
| 15 | 24.6 | 26.70% |
| 16 | 25.2 | 22.30% |
| 17 | 25.9 | 13.90% |
| 18 | 26.5 | 7.40% |
| 19 | 27.0 | 8.60% |
| 20 | 29.2 | 7.00% |
| 21 | 33.2 | 7.80% |
| 22 | 34.8 | 6.50% |
| 23 | 37.2 | 3.00% |
| 24 | 38.8 | 3.40% |

### Method of Treatment

Disclosed herein is a method of treating a disease or disorder in a subject, the method comprising administering to the subject a crystalline form disclosed herein, wherein the disease or disorder is inflammatory bowel disease (IBD). In some embodiments, the disease or disorder is ulcerative colitis (“UC”) or Crohn's disease (“CD”). In some embodiments, the disease or disorder is ulcerative colitis (“UC”). In some embodiments, the disease or disorder is Crohn's disease (“CD”).

### Inflammatory Bowel Disease (IED)

IBD is an umbrella term used to describe disorders that involve chronic inflammation of the digestive tract. Types of IBD include ulcerative colitis (“UC”) and Crohn's disease (“CD”). IBD symptoms vary and depend on the severity of inflammation and the location it occurs. According to GlobalData, in 2019, there were 1.7 million diagnosed UC patients in 8 major markets (US, 5EU, Japan and Canada) and the market sales reached $6.8 billion in that year. [In addition, there were 1.3 million UC diagnosed prevalent population in 8 major markets (US, 5EU, Japan and Canada) and the market sales reach $7.4 billion.]

Inflammatory bowel diseases are characterized by repeated inflammation and wounding of the mucosa and loss of the intestinal epithelial barrier function, which lead to the passage of bacteria or bacterial products from the gut lumen to the serosa and into the blood, resulting in systemic bacteremia and endotoxemia. PHD inhibition has been shown to reduce disease severity in murine models of colitis on several levels of clinical scoring. The proposed mechanism for the therapeutic activity of PHD inhibitors is through HIF-la stabilization, which drives epithelial barrier augmentation and healing.

Despite the efficacy of current treatment with anti-inflammation agents or immune-suppressive agents, a large fraction of IBD patients do not respond adequately to currently available therapies and do not achieve long-term remission. Inhibitors of PHDs may provide a new therapeutic option for IBD and may be combined with available anti-inflammatory drugs to achieve an enhanced efficacy.

### Dosing

In certain embodiments, the compositions containing the crystalline form described herein are administered for prophylactic and/or therapeutic treatments. In certain therapeutic applications, the compositions are administered to a patient already suffering from a disease or condition, in an amount sufficient to cure or at least partially arrest at least one of the symptoms of the disease or condition. Amounts effective for this use depend on the severity and course of the disease or condition, previous therapy, the patient's health status, weight, and response to the drugs, and the judgment of the treating physician. Therapeutically effective amounts are optionally determined by methods including, but not limited to, a dose escalation and/or dose ranging clinical trial.

In prophylactic applications, compositions containing the crystalline form described herein are administered to a patient susceptible to or otherwise at risk of a particular disease, disorder, or condition. Such an amount is defined to be a “prophylactically effective amount or dose.” In this use, the precise amounts also depend on the patient's state of health, weight, and the like. When used in patients, effective amounts for this use will depend on the severity and course of the disease, disorder or condition, previous therapy, the patient's health status and response to the drugs, and the judgment of the treating physician. In one aspect, prophylactic treatments include administering to a mammal, who previously experienced at least one symptom of or risk factor for the disease being treated and is currently in remission, a pharmaceutical composition comprising a compound described herein, or a pharmaceutically acceptable salt thereof, in order to prevent a return of the symptoms of the disease or condition.

In any of the aforementioned aspects are further embodiments in which the effective amount of the crystalline form described herein, or a pharmaceutically acceptable salt thereof, is: (a) systemically administered to the mammal; and/or (b) administered orally to the mammal; and/or (c) intravenously administered to the mammal; and/or (d) administered by injection to the mammal; and/or (e) administered topically to the mammal; and/or (f) administered non-systemically or locally to the mammal.

### Definitions

Unless otherwise stated, the following terms used in this application have the definitions given below. The use of the term “including” as well as other forms, such as “include,” “includes,” and “included,” is not limiting. The section headings used herein are for organizational purposes only and are not to be construed as limiting the subject matter described.

The term “acceptable” with respect to a formulation, composition, or ingredient, as used herein, means having no persistent detrimental effect on the general health of the subject being treated.

The terms “administer,” “administering,” “administration,” and the like, as used herein, refer to the methods that may be used to enable delivery of compounds or compositions to the desired site of biological action. These methods include, but are not limited to oral routes, intraduodenal routes, parenteral injection (including intravenous, subcutaneous, intraperitoneal, intramuscular, intravascular or infusion), topical and rectal administration. Those of skill in the art are familiar with administration techniques that can be employed with the compounds and methods described herein. In some embodiments, the compounds and compositions described herein are administered orally.

The terms “effective amount” or “therapeutically effective amount,” as used herein, refer to a sufficient amount of an agent or a compound being administered, which will relieve to some extent one or more of the symptoms of the disease or condition being treated. The result includes reduction and/or alleviation of the signs, symptoms, or causes of a disease, or any other desired alteration of a biological system. For example, an “effective amount” for therapeutic uses is the amount of the composition comprising a compound as disclosed herein required to provide a clinically significant decrease in disease symptoms. An appropriate “effective” amount in any individual case is optionally determined using techniques, such as a dose escalation study.

The terms “enhance” or “enhancing,” as used herein, means to increase, or prolong either in potency or duration a desired effect. Thus, in regard to enhancing the effect of therapeutic agents, the term “enhancing” refers to the ability to increase or prolong, either in potency or duration, the effect of other therapeutic agents on a system. An “enhancing-effective amount,” as used herein, refers to an amount adequate to enhance the effect of another therapeutic agent in a desired system.

The term “subject” or “patient” encompasses mammals. Examples of mammals include, but are not limited to, any member of the Mammalian class: humans, non-human primates such as chimpanzees, and other apes and monkey species; farm animals such as cattle, horses, sheep, goats, swine; domestic animals such as rabbits, dogs, and cats; laboratory animals including rodents, such as rats, mice and guinea pigs, and the like. In one aspect, the mammal is a human.

The terms “treat,” “treating” or “treatment,” as used herein, include alleviating, abating or ameliorating at least one symptom of a disease or condition, preventing additional symptoms, inhibiting the disease or condition, e.g., arresting the development of the disease or condition, relieving the disease or condition, causing regression of the disease or condition, relieving a condition caused by the disease or condition, or stopping the symptoms of the disease or condition either prophylactically and/or therapeutically.

The term “about” means within a statistically meaningful range of a value, such as a stated concentration range, time frame, molecular weight, particle size, temperature, or pH. Such a range can be within an order of magnitude, typically within 10%, more typically within 5%, and even more typically within 3% of the indicated value or range. Sometimes, such a range can be within the experimental error typical of standard methods used for the measurement and/or determination of a given value or range. The allowable variation encompassed by the term “about” will depend upon the particular system under study, and can be readily appreciated by one of ordinary skill in the art. Whenever a range is recited within this application, every whole number integer within the range is also contemplated as an embodiment of the disclosure. In the context of the disclosure, when used or whether or not used the word, such as “about,” it means that within a given value or range of 10%, appropriately within 5%, especially within 1%.

If multiple diffraction patterns are available, then assessments of particle statistics (PS) and/or preferred orientation (PO) are possible. Consistency of relative intensity among XRPD patterns from multiple diffractometers indicates good orientation statistics. Alternatively, the observed XRPD pattern may be compared with a calculated XRPD pattern based upon a single crystal structure, if available. Two-dimensional scattering patterns using area detectors can also be used to evaluate PS/PO. If the effects of both PS and PO are determined to be negligible, then the XRPD pattern is representative of the powder average intensity for the sample and prominent peaks may be identified as “Representative Peaks.” In general, the more data collected to determine Representative Peaks, the more confident one can be of the classification of those peaks.

“Characteristic peaks,” to the extent they exist, are a subset of representative peaks and are used to differentiate one crystalline polymorph from another crystalline polymorph (polymorphs being crystalline forms having the same chemical composition). Characteristic peaks are determined by evaluating which representative peaks, if any, are present in one crystalline polymorph of a compound against all other known crystalline polymorphs of that compound to within ±0.2° 2Θ. Not all crystalline polymorphs of a compound necessarily have at least one characteristic peak.

The term “preferred orientation” as used herein refers to an extreme case of non-random distribution of the crystallites of a solid state form. In XRPD, the ideal sample is homogenous and the crystallites are randomly distributed in the bulk solid. In a truly random sample, each possible reflection from a given set of planes will have and equal number of crystallites contributing to it. However, when the solid state form is in a preferred orientation this is not the case. Accordingly, comparing the intensity between a randomly oriented diffraction pattern and a preferred oriented diffraction pattern can look entirely different. Quantitative analysis depending on intensity ratios are greatly distorted by preferred orientation. Careful sample preparation is important for decreasing the incidence of a preferred orientation.

The term “substantially the same,” as used herein to reference a figure is intended to mean that the figure is considered representative of the type and kind of characteristic data that is obtained by a skilled artisan in view of deviations acceptable in the art. Such deviations may be caused by factors related to sample size, sample preparation, particular instrument used, operation conditions, and other experimental condition variations known in the art. For example, one skilled in the art can appreciate that the endotherm onset and peak temperatures as measured by differential scanning calorimetry (DSC) may vary significantly from experiment to experiment. For example, one skilled in the art can readily identify whether two X-ray diffraction patterns or two DSC thermograms are substantially the same. In some embodiments, when characteristic peaks of two X-ray diffraction patterns do not vary more than ±0.2° 2θ, it is deemed that the X-ray diffraction patterns are substantially the same.

As used herein, salts of Compound 1 (e.g., HCl salt, sulfate salt, methanesulfonate salt, benzenesulfonate salt, and naphthalene-1,5-disulfonate salt) include compounds where the corresponding acid is in an ionized, non-ionized, associated, or unassociated form. In some embodiments, the corresponding acid is in an ionized and/or associated forms. In some embodiments, the corresponding acid is in a nonionized and/or unassociated forms. Salts of Compound 1 also include mono-acid, di-acid, etc. forms of the salts.

### EXAMPLES

The following examples are provided for illustrative purposes only and not to limit the scope of the claims provided herein.

### Example 1: PHD2 Enzymatic Assay Procedure

Compound DMSO stock preparation: Compound 1 was reconstituted into 20 mM stock by DMSO.

Compound storage: Compound 1 in DMSO was stored at RT in a desiccator for short-term storage (up to 3 months).

### Working Stock Preparation:

Reference Roxadustat (FG-4592) was 3-fold serial diluted from 400 μM for 10 doses in DMSO.
The compounds were 3-fold serial diluted from 400 μM for 10 doses in DMSO.
Prepared 200× positive control (400 μM, FG-4592) and 200× vehicle control (100% DMSO).
Centrifuged compound plates at 1000 rpm for 1 min.

### Compound Screening:

a) Transferred 40 nl compound dilutions into each well of assay plates using Echo 655;
b) Sealed the assay plate and centrifuge compound plates at 1000 rpm for 1 min.
c) Prepared and add 4 μL of the 2×PHD2 enzyme working solution to individual well of the assay plate.
d) Sealed the assay plate and centrifuge compound plates at 1000 rpm for 1 min. Incubate plate at RT for 30 min.
e) Prepared and add 4 μl 2×PHD2 substrate working solution to each well of the assay plate.
f) Prepared and added 4 μL 4× stop solution to the each well of the assay plate.
g) Prepared 4× detection solution with AlphaScreen Streptavidin Donor beads, AlphaScreen Protein A Acceptor beads and Hydroxy-HIF-la (Pro564) (D43B5) XP® Rabbit mAb.
h) Added 4 μL 4× detection solution to the each well of the assay plate, repeat at step d.
i) Read Alphascreen signal on Envision HTS plate reader.

### Data Analysis

ALPHASCREEN signal (ALPcmpd) is calculated for each well
% Inhibition is calculated as follow:

$$\% Inhibition = [ 1 - \frac{{\overline{ALP}}_{compound} - {\overline{ALP}}_{positive}}{{\overline{ALP}}_{vehicle} - {\overline{ALP}}_{positive}} ] \times 100$$

A̅L̅P̅ₚₒₛᵢₜᵢᵥₑ: The average ALP for the positive controls across the plate.
A̅L̅P̅_{vehicle}: The average ALP for the negative controls across the plate.
Calculate IC₅₀ and Plot effect-dose curve of compounds:
Calculated IC₅₀ by fitting % inhibition values and log of compound concentrations to nonlinear regression (dose response−variable slope) with Graphpad 8.0.

$$Y = Bottom + ( Top - Bottom ) / ( 1 + 10 \hat{} ( ( Log IC 50 - X ) * HillSlope ) )$$

X: log of Inhibitor concentration; Y: % Inhibition.

### Example 2: EPO Elisa Assay

The compounds powder were dissolved in 100% DMSO. The compounds stock solution were kept in nitrogen cabinet.

### Experimental Methods

Cell seeding: Added 100 μl cell suspension contain 20 k Hep3B cell per well. Preparation of compound concentration gradient: Compound 1 at top dose of 100 μM, 3-fold dilution, 8 doses, singlet or duplicate. Prepare a solution of 200× the final concentration in a 96-well plate, dilute the compound by 200/3× with cell culture medium, and then pipette 50 μL to wells. Add 50 μL of culture medium containing DMSO to the minimum control well to make the final concentration contain 5%. DMSO, and add 50 μL of the highest concentration of reference compound to the maximum control well, and incubate at 37 [symbol] to 24 h.
Washed the reaction plate twice with 400 μL of 1× Wash Buffer per well.
Added 100 μL of the diluted standard (including standard blank control) to the appropriate wells.
Added 50 μL of sample and 50 μL of Sample Diluent to the sample well.
Added 50 μL 1× Biotin Conjugated Antibody to all wells and incubate for 1 hour at room temperature.
Washed the reaction plate 6 times with 400 μL 1× Wash Buffer per well.
Added 100 μL 1× Streptavidin-HRP to each well. Incubate at room temperature for 15 minutes.
Washed the reaction plate 6 times with 400 μL 1× Wash Buffer per well.
Added 100 μL TMB Substrate Solution to each well. Incubate at room temperature for 10 minutes.
Added 100 μL Stop Solution to each well.
Read OD450 with EnSight.

### Data Analysis

Using GraphPad Prism 5.

$$\% Act . = ( Compound signal - Min signal ) / ( Max signal - Min signal ) * 100.$$

Max signal was obtained from the maximum control wells.

Min signal was obtained from the minimum control wells.

Take the log value of the concentration as the X-axis, and the percentage inhibition rate on the Y-axis. Use the analysis software GraphPad Prism 5 log(inhibitor) vs. response −Variable slope to fit the dose-response curve to obtain the EC₅₀ value of each compound.

The data from examples 1 and 2 are shown below.

| Ex. | PHD2 | Caco2-HIF1α-HiBit assay-EC₅₀ |
|---|---|---|
| Compound 1 | A | A |

PHD2 (nM): 0<A≤5; 5<B≤20; 20<C≤100; 100<D≤1,000; 1,000<E<100,000
Caco2-HIF1α-HiBit assay (EC₅₀, nM): 0<A≤2,500; 2,500<B≤5,000; 5,000<C≤7500; 7,500<D≤10,000; 10,000<E≤100,000

### Example 3: Preparation of Compound 1 Freebase Type A

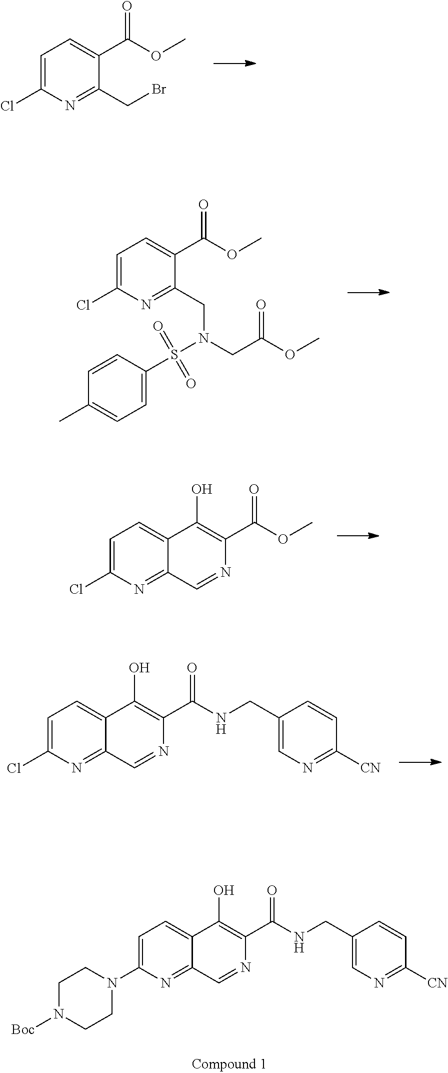

To a solution of methyl 2-(bromomethyl)-6-chloronicotinate (5 g, 18.9 mmol) and methyl 2-(p-tolylsulfonylamino) acetate (4.6 g, 18.9 mmol) in DMF (50 mL) was added K₂CO₃ (5.02 g, 47.4 mmol) and NaI (0.28 g, 1.86 mmol). The mixture was stirred at 50 [symbol] for 12 h under N₂ atmosphere. The reaction mixture was diluted with ethyl acetate (200 mL) and washed with H₂O (80 mL×3). The organic layer was washed with brine (80 mL×3), dried over MgSO₄, filtered, and concentrated under reduced pressure to give a residue. The residue was purified by silica gel chromatograph to afford methyl 6-chloro-2-(((N-(2-methoxy-2-oxoethyl)4-methylphenyl)sulfonamido)methyl)nicotinate (6 g, crude) as a yellow solid.

To a solution of methyl 6-chloro-2-(((N-(2-methoxy-2-oxoethyl)-4-methylphenyl)sulfonamido)methyl)nicotinate (6 g, 14 mmol) in DMSO (60 mL) was added K₂CO₃ (11.6 g, 84.3 mmol). The mixture was stirred at 50 [symbol] for 4 h under N₂ atmosphere. The mixture was diluted with H₂O (60 mL) and the aqueous was adjusted pH to 6 with 1 M HCl. The precipitated solid was filtered and dried to afford methyl 2-chloro-5-hydroxy-1,7-naphthyridine-6-carboxylate (1.5 g, 45% yield) as an off-white solid. ¹H NMR (400 MHz, DMSO-d₆) δ 8.86 (s, 1H), 8.72 (d, J=0.9 Hz, 1H), 7.91 (d, J=8.8 Hz, 1H), 3.95 (s, 3H).

To a solution of methyl 2-chloro-5-hydroxy-1,7-naphthyridine-6-carboxylate (1 g, 4.19 mmol) in MeOH (30 mL) were added 5-(aminomethyl)pyridine-2-carbonitrile (0.84 g, 6.29 mmol), TEA (2.91 mL, 20.95 mmol), and the reaction was stirred at 75 [symbol] overnight. The reaction mixture was filtered through normal funnel and the filter cake was washed with 10 mL MeOH, dried in vacuum to afford 2-chloro-N-((6-cyanopyridin-3-yl)methyl)-5-hydroxy-1,7-naphthyridine-6-carboxamide (1.1 g, 3.24 mmol, 77% yield) as a yellow solid.

To a solution of 2-chloro-N-((6-cyanopyridin-3-yl)methyl)-5-hydroxy-1,7-naphthyridine-6-carboxamide (1.1 g, 3.24 mmol) in DMSO (15 mL) were added TEA (1.35 mL, 9.71 mmol), tert-butyl piperazine-1-carboxylate (904 mg, 4.86 mmol), and the reaction was stirred at 100 [symbol] for 2 h under N₂. The reaction was cooled and poured into water H₂O (200 mL). The mixture was extracted with EtOAc (50 mL×3). The combined organic layer was washed with saturated NaCl solution (30 mL×3), and concentrated in vacuo. The residue was triturated with CH₃CN (20 mL) and CH₂Cl₂ (5 mL) and filtered to afford Compound 1 (500 mg, 1.02 mmol, 32% yield) as a white solid. LCMS: RT=1.838 min; MS m/z (ESI)[M+H]⁺=490.1. 1H NMR (400 MHz, DMSO-d₆) δ 13.36 (s, 1H), 9.77 (t, J=6.3 Hz, 1H), 8.76 (s, 1H), 8.44 (s, 1H), 8.29 (d, J=9.4 Hz, 1H), 8.00 (s, 2H), 7.48 (d, J=9.5 Hz, 1H), 4.63 (d, J=6.3 Hz, 2H), 3.82-3.79 (m, 4H), 3.50-3.48 (m, 4H), 1.46 (s, 9H).

Approximate 50 mg of the obtained solid (Compound 1) was equilibrated in 0.4-0.5 mL of methanol/ethanol/isopropanol/ethyl acetate/the solvent mixture of dimethyl sulfoxide and water (1v:1v) at 25 [symbol] for 2 weeks, or in the solvent mixture of dimethyl sulfoxide and water (1v:1v) at 50 [symbol] for 1 week. The solid precipitate was collected for XRPD, DSC, TGA and ¹H-NMR.

The XRPD pattern of Compound 1 freebase Type A is shown in FIG. 1. Major peaks and their related intensities in the XRPD pattern are shown in Table 1. Type A is a mixture of Compound 1 freebase Type B and Type D.

### Characterization Method:

DSC analysis: TA Discovery 2500, 30-250 T or before decomposition, heating rate 10 [symbol] /min.

TGA analysis: Discovery 5500, 300 [symbol] or abort next segment if weight <80% (w/w), heating rate 10 [symbol] /min.

XRPD: Bruker D8 Advance X-ray powder diffractometer with Cu/K-Alpha1 radiation. The tube voltage was 40 kV. The tube current was 40 mA. Scan range was from 3 to 40 degree 2-theta. The step size was 0.02° at a scanning speed of 0.12 second/step.

DSC and TGA results shown in FIG. 2 and FIG. 3 indicates Compound 1 freebase Type A has an onset of endothermal event at around 223.5 [symbol] .

### Example 4: Alternative Preparation of Compound 1 Freebase Type A

Approximate 50 mg of the obtained solid of Compound 1 was equilibrated in 0.4-0.5 mL of ethyl acetate/2-methyl tetrahydrofuran/ethanol/isopropanol/the solvent mixture of dimethyl sulfoxide and water (1v:1v) under a temperature cycle between 50 [symbol] and 5 [symbol] at a cooling/heating rate of 0.1 [symbol] /min for 10 cycles. The temperature cycle started from 50 [symbol] . The solid precipitate was collected for XRPD, DSC, and TGA. The XRPD pattern of solid was same as that in Table 1 and confirmed to be Compound 1 freebase Type A.

### Example 5: Preparation of Compound 1 Freebase Type 1

Approximate 50 mg of Compound 1 freebase Type A was equilibrated in 0.4-0.5 mL of acetone/methylethylketone/tetrahydrofuran/acetonitrile/the solvent mixture of dimethylformamide and isopropyl acetate (1v:1v)/the solvent mixture of dimethylacetamide and water (95v:5v) at 25 [symbol] for 2 weeks, or in methylethylketone/tetrahydrofuran/acetonitrile/the solvent mixture of dimethylacetamide and water (95v:5v) at 50 [symbol] for 1 week. The solid precipitate was collected for XRPD, DSC, and TGA.

The XRPD pattern of Compound 1 freebase Type 1 is shown in FIG. 4. Major peaks and their related intensities in the XRPD pattern are shown in Table 2. Compound 1 freebase Type 1 prepared in this method is an acetone solvate, methylethylketone solvate, tetrahydrofuran solvate, acetonitrile solvate, dimethylformamide solvate, or a dimethylacetamide solvate of Compound 1.

### Characterization Methods:

XRPD: Bruker D8 Advance X-ray powder diffractometer with Cu/K-Alpha1 radiation. The tube voltage was 40 kV. The tube current was 40 mA. Scan range was from 3 to 40 degree 2-theta. The step size was 0.02° at a scanning speed of 0.12 second/step.

DSC analysis: TA Discovery 2500, 30-250 [symbol] or before decomposition, heating rate 10 [symbol] /min. TGA analysis: Discovery 5500, 300 [symbol] or abort next segment if weight <80% (w/w), heating rate 10 [symbol] /min.

DSC and TGA results shown in FIG. 5 and FIG. 6 indicate Compound 1 freebase Type 1 has an onset of endothermal event at around 124.8 [symbol] and onset melting temperature at around 231.7 [symbol] .

### Residual Solvent Information of Type 1

| | Type 1 | | | | | | | |
|---|---|---|---|---|---|---|---|---|
| | | | Acetone | | | | Ethyl | 2- |
| Sample | MEK | THF | solvate + | ACN | DMAc | DMF | Acetate | MeTHF |
| ID | solvate | solvate | Type C | solvate | solvate | solvate | solvate | solvate |
| Residual | 3.4% | 8.8% | 3.4% | 3.3% | 11.2% | 6.6% | // | 7.0% |
| solvent | (0.24 | (0.66 | (0.30 | (0.41 | (0.71 | (0.47 | | (0.43 |
| (by ¹H- | equiv.) | equiv.) | equiv.) | equiv.) | equiv.) | equiv.) | | equiv.) |
| NMR) | MEK | THF | acetone | ACN | DMAc | DMF | | 2-MeTHF |
| | residue | residue | residue | residue | residue | residue | | residue |

### Example 6: Alternative Preparation of Compound 1 Freebase Type 1

Approximate 50 mg of Compound 1 freebase Type A was equilibrated in 0.4-0.5 mL of acetone/methylethylketone/tetrahydrofuran/acetonitrile/the solvent mixture of dimethylformamide and isopropyl acetate (1v:1v)/the solvent mixture of dimethylacetamide and water (95v:5v) under a temperature cycle between 50 T and 5 [symbol] at a cooling/heating rate of 0.1 [symbol] /min for 10 cycles. The temperature cycle started from 50 [symbol] . The solid precipitate was collected for XRPD test. The XRPD pattern of solid was same as that in Table 2 and confirmed to be Compound 1 freebase Type 1. Compound 1 freebase Type 1 prepared in this method is an acetone solvate, methylethylketone solvate, tetrahydrofuran solvate, acetonitrile solvate, dimethylformamide solvate, or a dimethylacetamide solvate of Compound 1.

### Example 7: Alternative Preparation of Compound 1 Freebase Type 1

Approximate 20-30 mg of Compound 1 freebase Type A was dissolved in 1.5-6 mL acetone/methylethylketone/tetrahydrofuran/2-methyltetrahydrofuran and filtered. The filtrates were slowly evaporated in ambient condition (about 20-25 [symbol] , 20-30% RH). After precipitation happened, the solid was collected by filtration for XRPD test. The XRPD pattern of solid was same as that in Table 2 and confirmed to be Compound 1 freebase Type 1. Compound 1 freebase Type 1 prepared in this method is an acetone solvate, methylethylketone solvate, tetrahydrofuran solvate, or a 2-methyltetrahydrofuran solvate of Compound 1.

### Example 8: Alternative Preparation of Compound 1 Freebase Type 1

Approximate 30 mg of Compound 1 freebase Type A was dissolved in the minimal volume of acetone/methylethylketone/tetrahydrofuran/2-methyltetrahydrofuran at 50 [symbol] and filtered. The filtrates were cooled to 5 [symbol] at 0.1 [symbol] /min and stirred until enough precipitates were obtained. Samples without precipitates at 5 [symbol] were further cooled to −20 [symbol] for precipitation. The solids were collected for XRPD test. The XRPD pattern of the solid was same as that in Table 2 and confirmed to be Compound 1 freebase Type 1. Compound 1 freebase Type 1 prepared in this method is an acetone solvate, methylethylketone solvate, tetrahydrofuran solvate, or a 2-methyltetrahydrofuran solvate of Compound 1.

### Example 9: Alternative Preparation of Compound 1 Freebase Type 1

Approximate 30 mg of Compound 1 freebase Type A was dissolved in the minimal volume of acetone/methylethylketone/ethylacetate/tetrahydrofuran/2-methyltetrahydrofuran at 50 [symbol] and filtered. The filtrates were cooled and stirred in an ice bath at 0 [symbol] . The precipitates were collected for XRPD test. The XRPD pattern of the solid was same as that in Table 2 and confirmed to be Compound 1 freebase Type 1. Compound 1 freebase Type 1 prepared in this method is an acetone solvate, methylethylketone solvate, ethyl acetate solvate, tetrahydrofuran solvate, or a 2-methyltetrahydrofiran solvate of Compound 1.

### Example 10: Alternative Preparation of Compound 1 Freebase Type 1

Approximate 25-40 mg of Compound 1 freebase Type A was dissolved in the minimal volume of 2-methyltetrahydrofuran at ambient temperature (20-25 [symbol] ) and filtered, followed by slow charging of 1-4 folds of acetonitrile into the filtrate. The precipitates were collected for XRPD analysis. The XRPD pattern of the solid was same as that in Table 2 and confirmed to be Compound 1 freebase Type 1.

### Example 11: Alternative Preparation of Compound 1 Freebase Type 1

Approximate 25-40 mg Compound 1 freebase Type A was dissolved in the minimal volume of tetrahydrofuran at ambient temperature (20-25 [symbol] ) and filtered. Approximate 1-4 folds of isopropyl acetate was slowly charged into the filtrate and cooled to 5 [symbol] . After equilibration for 3 days at 5 [symbol] C, the precipitates were collected for XRPD analysis. The XRPD patter of the solid was same as that in Table 2 and confirmed to be Compound 1 freebase Type 1.

### Example 12: Preparation of Compound 1 Freebase Type B

Compound 1 freebase Type B was obtained by heating methylethylketone solvate, tetrahydrofuran solvate, acetone solvate, or acetonitrile solvate of Compound 1 freebase Type 1 at 150 [symbol] . The XRPD pattern of Compound 1 freebase Type B is shown in FIG. 7. Major peaks and their related intensities in the XRPD pattern are shown in Table 3. DSC and TGA results shown in FIG. 8 and FIG. 9 indicates Compound 1 freebase Type B has an onset of endothermal event at around 232.9 [symbol] . No solvent residue was found in Compound 1 freebase Type B. Thus, Compound 1 freebase Type B is a polymorph of Compound 1.

### Characterization Method:

XRPD: Bruker D8 Advance X-ray powder diffractometer with Cu/K-Alpha1 radiation. The tube voltage was 40 kV. The tube current was 40 mA. Scan range was from 3 to 40 degree 2-theta. The step size was 0.02 [symbol] at a scanning speed of 0.12 second/step.

DSC analysis: TA Discovery 2500, 30-250 [symbol] or before decomposition, heating rate 10 [symbol] /min. TGA analysis: Discovery 5500, 300 [symbol] or abort next segment if weight <80% (w/w), heating rate 10 [symbol] /min.

### Example 13: Preparation of Compound 1 Freebase Type C

Approximate 25-40 mg of Compound 1 freebase Type A was dissolved in the minimal volume of acetone (0.5 mL) at ambient temperature (20-25 [symbol] ) and filtered, followed by slow charging of 1-4 folds of water into the filtrate. The precipitates were collected for XRPD DSC, TGA, ¹HNMR, and DVS tests.

The XRPD pattern of Compound 1 freebase Type C is shown in FIG. 10. Major peaks and their related intensities in the XRPD pattern are shown in Table 4.

### Characterization Method:

DSC analysis: TA Discovery 2500, 30-250 [symbol] or before decomposition, heating rate 10 [symbol] /min.

TGA analysis: Discovery 5500, 300 [symbol] or abort next segment if weight <80% (w/w), heating rate 10 [symbol] /min.

DVS analysis: ProUmind High-throughput DVS. The relative humidity cycle was 40-95-0-95-40% with the stage step of 10% and equilibrium time of 240 min. The total gas flow was 4000 sccm. Water worked as the solvent. The oven temperature was 25 [symbol] .

XRPD: Bruker D8 Advance X-ray powder diffractometer with Cu/K-Alpha1 radiation. The tube voltage was 40 kV. The tube current was 40 mA. Scan range was from 3 to 40 degree 2-theta. The step size was 0.02° at a scanning speed of 0.12 second/step.

DSC result shown in FIG. 11 indicates Compound 1 freebase Type C has an onset of endothermal event at around 231.2 [symbol] . FIG. 12 shows a water pick-up about 0.2% at 80% relative humidity at 25 [symbol] . No solvent residue was found in Compound 1 freebase Type C. Compound 1 freebase Type C is an anhydrate polymorph of Compound 1.

### Example 14: Alternative Preparation of Compound 1 Freebase Type C

Approximate 50 mg of Compound 1 freebase Type A was equilibrated in 0.4-0.5 mL of methanol under a temperature cycle between 50 [symbol] and 5 [symbol] at a cooling/heating rate of 0.1 [symbol] /min for 10 cycles. The temperature cycle started from 50 [symbol] . The solid precipitate was collected for XRPD test. The XRPD pattern of the solid was same as that in Table 4 and confirmed to be Compound 1 freebase Type C.

### Example 15: Alternative Preparation of Compound 1 Freebase Type C

Approximate 50 mg of Compound 1 freebase Type A was equilibrated in 0.4-0.5 mL of methanol/the solvent mixture of dimethylformamide and isopropyl acetate (1v:1v) at 50 [symbol] for 1 week. The solid precipitate was collected for XRPD test. The XRPD pattern of the solid was same as that in Table 4 and confirmed to be Compound 1 freebase Type C.

### Example 16: Alternative Preparation of Compound 1 Freebase Type C

Approximate 1 g of mixture of Compound 1 freebase Type B, freebase Type D, and freebase Type 1 was triturated in 30 mL ethanol at 50 [symbol] for 7 days. The solid was isolated and vacuum dried at 50 [symbol] for 4 hours. The XRPD test result of the solid was same as that in Table 4 and confirmed to be Compound 1 freebase Type C.

### Example 17: Preparation of Compound 1 Freebase Type D

Approximate 50 mg of Compound 1 freebase Type A was equilibrated in 0.4-0.5 mL of ethanol/isopropanol/ethyl acetate/2-methyltetrahydrofuran at 50 [symbol] for 1 week. The solid precipitate was collected for XRPD, DSC, and TGA.

The XRPD pattern of Compound 1 freebase Type D is shown in FIG. 13. Major peaks and their related intensities in the XRPD pattern are shown in Table 5. Compound 1 freebase Type D is an anhydrate of Compound 1.

### Characterization Method:

XRPD: Bruker D8 Advance X-ray powder diffractometer with Cu/K-Alpha1 radiation. The tube voltage was 40 kV. The tube current was 40 mA. Scan range was from 3 to 40 degree 2-theta. The step size was 0.02° at a scanning speed of 0.12 second/step.

DSC analysis: TA Discovery 2500, 30-250 T or before decomposition, heating rate 10 [symbol] /min.

TGA analysis: Discovery 5500, 300 TC or abort next segment if weight <80% (w/w), heating rate 10 [symbol] /min.

DSC and TGA results shown in FIG. 14 and FIG. 15 indicate Compound 1 freebase Type D has an onset of endothermal event at around 194.3 [symbol] .

### Example 18: Alternative Preparation of Compound 1 Freebase Type D

Approximate 20-30 mg of Compound 1 freebase Type A was dissolved in 1.5-6 mL ethanol and filtered, followed by the slow evaporation of the filtrate at ambient condition (about 20-25 [symbol] C, 20-30% RH). After precipitation happened, the solid was collected for XRPD test. The XRPD pattern of solid was same as that in Table 5 and confirmed to be Compound 1 freebase Type D.

### Example 19: Alternative Preparation of Compound 1 Freebase Type D

Approximate 25-40 mg of Compound 1 freebase Type A was dissolved in the minimal volume of tetrahydrofuran at ambient temperature (20-25 [symbol] ) and filtered, followed by slow charging of 1-4 folds of methyl tertiary-butyl ether into the filtrate. The precipitates were collected for XRPD test. The XRPD pattern of the solid was same as that in Table 5 and confirmed to be Compound 1 freebase Type D.

### Example 20: Alternative Preparation of Compound 1 Freebase Type D

Approximate 25-40 mg of Compound 1 freebase Type A was dissolved in the minimal volume of 2-methyltetrahydrofuran at ambient temperature (20-25 T) and filtered, followed by slow charging of 1-5 folds of heptane into the filtrate. The precipitates were collected for XRPD test. The XRPD pattern of the solid was same as that in Table 5 and confirmed to be Compound 1 freebase Type D.

### Example 21: Preparation of Compound 1 Freebase Type E

Approximate 20-30 mg of Compound 1 freebase Type A was dissolved in dichloromethane and filtered, followed by the slow evaporation of the filtrate at ambient condition (about 20-25 T, 20-30% RH). After precipitation happened, the solid was collected for XRPD, DSC, and TGA test. The XRPD pattern of Compound 1 freebase Type E is shown in FIG. 16. Major peaks and their related intensities in the XRPD pattern are shown in Table 6. Compound 1 freebase Type E is an anhydrate of Compound 1.

### Characterization Method:

XRPD: Bruker D8 Advance X-ray powder diffractometer with Cu/K-Alpha1 radiation. The tube voltage was 40 kV. The tube current was 40 mA. Scan range was from 3 to 40 degree 2-theta. The step size was 0.0 [symbol] a scan nig speed of 0.12 second/step.

DSC analysis: TA Discovery 2500, 30-250 [symbol] or before decomposition, heating rate 10 [symbol] /min.

TGA analysis: Discovery 5500, 300 [symbol] or abort next segment if weight <80% (w/w), heating rate 10 [symbol] /min.

DSC and TGA results shown in FIG. 17 and FIG. 18 indicate Compound 1 freebase Type E has an onset of endothermal event at around 224.2 [symbol] .

### Example 22: Preparation of Compound 1 Freebase Type F

Approximate 50 mg of the mixture of Compound 1 freebase Type B, Type D, and Type 1 was charged into 4 mL dichloromethane at 25 [symbol] and stirred, followed by filtration. The filtrate was set on slow evaporation at room temperature for 2-7 days. The precipitated solid was collected for XRPD, DSC, and TGA test.

The XRPD pattern of Compound 1 freebase Type F is shown in FIG. 19. Major peaks and their related intensities in the XRPD pattern are shown in Table 7. Compound 1 freebase Type F is an anhydrate of Compound 1.

### Characterization Method:

XRPD: Bruker D8 Advance X-ray powder diffractometer with Cu/K-Alpha1 radiation. The tube voltage was 30 kV. The tube current was 10 mA. Scan range was from 3 to 40 degree 2-theta. The step size was 0.02° at a scanning speed of 0.2 second/step.

DSC analysis: Discovery DSC 250, 25-250 [symbol] or before decomposition, heating rate 10 [symbol] /min.

TGA analysis: Discovery 550, 300 T or abort next segment if weight <80/a (w/w), heating rate 10 [symbol] /min.

DSC and TGA results shown in FIG. 20 and FIG. 21 indicate Compound 1 freebase Type F has an onset of exothermal event at 106.4 [symbol] and an onset of endothermal event at around 228.4 [symbol] .

### Example 23: Preparation of Compound 1 Compound 1HCl Salt Type A

Approximate 50 mg of Compound 1 freebase Type A and one or two equivalent of HCl were added in 1 mL methylethylketone or tetrahydrofuran. Obtained mixtures were stirred at 50 T for 2 hours and then at 25 T for 48 hours. Obtained suspension were filtered through a 0.45 in nylon membrane filter by centrifugation at 14,000 rpm. After being dried at 50 T under vacuum for about 2 h, the solids were characterized by XRPD, DSC, TGA, ¹H-NMR, IC, and KF. Compound 1 HCl salt Type A is an anhydrate. Major peaks and their related intensities in the XRPD pattern are shown in Table 8.

The XRPD pattern of Compound 1 HCl salt Type A is shown in FIG. 22. DSC and TGA results are shown in FIG. 23 and FIG. 24.

### Example 24: Alternative Preparation of Compound 1 HCl Salt Type A

About 400 mg of Compound 1 freebase Type A, was weighed into a 20 mL glass vial, 3.3 mL of THF was added into the vial under stirring at 50 T for about 2 min.

1.0 equiv. of 1.2N HCl aqueous solution (0.73 mL, 0.1 mL of 12N HCl was diluted with 0.9 mL of THF) was added into the suspension slowly.

5-10 mg of HCl salt Type A seeds were added into the suspension. 2 mL of THF was added into the suspension to improve the mobility.

After kept stirring at 50 [symbol] for about 2 hours, the suspension was cooled to 25 [symbol] by natural cooling and kept stirring at 25 [symbol] .

Solids were collected by centrifuge filtration and then dried at 50 [symbol] under vacuum for about 2 hours.

About 375 mg HCl salt Type A was obtained as an off-white solid in a yield of 77.4%.

The solids were characterized by XRPD, DSC, TGA, HPLC, ¹H-NMR, and IC.

DSC shows a melting peak at Tₒₙₛₑₜ of 182.5 [symbol] . Decomposition occurs upon melting. TGA shows about 1.7% weight loss at about 160 [symbol] about 0.5% weight loss from about 160 [symbol] to 206 [symbol] . HPLC shows 97.5% chemical purity. IC shows the ratio of compound (I):HCl is 1:1.06. ¹H-NMR shows 1.3% THF residue by weight.

### Example 25: Preparation of Compound 1 Sulfate Salt Type A

Approximate 50 mg of Compound 1 freebase Type A and 1 equivalent of H₂SO₄ were added in 1 mL methylethylketone or tetrahydrofuran. Obtained mixtures were stirred at 50 [symbol] for 2 hours and then at 25 [symbol] for 48 hours. Obtained suspension were filtered through a 0.45 μm nylon membrane filter by centrifugation at 14,000 rpm. After being dried at 50 [symbol] under vacuum for about 2 h, the solids were characterized by XRPD, DSC, TGA, ¹H-NMR, IC, and KF. Compound 1 Sulfate salt Type A is an anhydrate.

Major peaks and their related intensities in the XRPD pattern are shown in Table 9.

The XRPD pattern of Compound 1 Sulfate salt Type A is shown in FIG. 25. DSC and TGA results are shown in FIG. 26 and FIG. 27.

### Example 26: Preparation of Compound 1 Sulfate Salt Type B

Approximate 50 mg of Compound 1 freebase Type A and 1 equivalent of H₂SO₄ were added in 1 mL methylethylketone. Obtained mixtures were stirred at 50 [symbol] for 2 hours and then at 25 [symbol] for 48 hours. Obtained suspension were filtered through a 0.45 μm nylon membrane filter by centrifugation at 14,000 rpm. After being dried at 50 [symbol] under vacuum for about 2 h, the solids were triturated in methylethylketone for 5 days. The solids were collected for XRPD, DSC, TGA, ¹H-NMR, IC, and KF test. The sulfate Type B is an anhydrate.

Major peaks and their related intensities in the XRPD pattern are shown in Table 10.

The XRPD pattern of Compound 1 Sulfate salt Type B is shown in FIG. 28. DSC and TGA results are shown in FIG. 29 and FIG. 30.

### Example 27: Alternative Preparation of Compound 1 Sulfate Salt Type B

About 400 mg of Compound 1 freebase Type A, was weighed into a 20 mL glass vial. 1 mL of MEK was added into the vial under stirring at 50 [symbol] for about 2 min.

1.0 equiv. of sulfuric acid (0.47 mL, 0.1 mL of concentrated sulfuric acid was diluted with 0.9 mL of MEK) was added into the suspension slowly.

5-10 mg of sulfate Type B seeds were added into the suspension. 3 mL of MEK was added into the suspension to improve the mobility.

After kept stirring at 50 [symbol] for about 2 hours, the suspension was cooled to 25 [symbol] by natural cooling and kept stirring at 25 [symbol] .

Solids were collected by centrifuge filtration and then dried at 50 [symbol] under vacuum for about 2 hours.

The solids were characterized by XRPD, DSC, TGA, HPLC, ¹H-NMR, and IC.

DSC shows a melting peak at Tₒₙₛₑₜ of 163.3 [symbol] . Decomposition occurs upon melting. TGA shows about 1.2% weight loss at about 150 [symbol] . HPLC shows 87.9% chemical purity. IC shows the ratio of compound (I):sulfuric acid is 1:1.23. ¹H-NMR shows 0.9% MEK residue by weight.

### Example 28: Preparation of Compound 1 methanesulfonate Type A

Approximate 50 mg of Compound 1 freebase Type A and 1 equivalent of methanesulfonic acid were added in 1 mL methylethylketone or tetrahydrofuran. Obtained mixtures were stirred at 50 [symbol] for 2 hours and then at 25 [symbol] for 48 hours. Obtained suspension were filtered through a 0.45 μm nylon membrane filter by centrifugation at 14,000 rpm. After being dried at 50 [symbol] under vacuum for about 2 h, the solids were characterized by XRPD, DSC, TGA, ¹H-NMR, IC, and KF. Major peaks and their related intensities in the XRPD pattern are shown in Table 11. Methanesulfonate Type A is an anhydrate.

The XRPD pattern of methanesulfonate Type A is shown in FIG. 31. DSC and TGA results are shown in FIG. 32 and FIG. 33.

### Example 29: Alternative Preparation of Methanesulfonate Type A

About 400 mg of Compound 1 freebase Type A was weighed into a 20 mL glass vial. 3 mL of THF was added into the vial under stirring at 50 [symbol] for about 2 min.

1.0 equiv. of methanesulfonic acid (0.56 mL, 0.1 mL of methanesulfonic acid was diluted with 0.9 mL of THF) was added into the suspension slowly.

5-10 mg of MSA salt Type A seeds were added into the suspension. Then 3 mL of THF was added into the suspension to improve the mobility.

After kept stirring at 50 [symbol] for about 2 hours, the suspension was cooled to 25 [symbol] by natural cooling and kept stirring at 25 [symbol] .

Solids were collected by centrifuge filtration and then dried at 50 [symbol] under vacuum for about 2 hours.

About 461 mg MSA salt Type A was obtained as a white solid in a yield of 95.4%.

The solids were characterized by XRPD, DSC, TGA, HPLC, and ¹H-NMR. DSC shows a melting peak at Tonset of 154.2 [symbol] . Decomposition occurs upon melting. TGA shows about 1.7% weight loss at about 140 [symbol] and about 16.7% weight loss from about 140 [symbol] to 184 [symbol] . HPLC shows 97.1% chemical purity. ¹H-NMR shows the ratio of compound (I):methanesulfonic acid is 1:1.04 and 2.0% THF residue by weight.

### Example 30: Preparation of Compound 1 benzenesulfonate Salt Type A

Approximate 50 mg of Compound 1 freebase Type A and 1 equivalent of benzenesulfonic acid were added in 1 mL methylethylketone or tetrahydrofuran. Obtained mixtures were stirred at 50 [symbol] for 2 hours and then at 25 [symbol] for 48 ours. Obtained suspension were filtered through a 0.45 μm nylon membrane filter by centrifugation at 14,000 rpm. After being dried at 50 [symbol] under vacuum for about 2 h, the solids were characterized by XRPD, DSC, TGA, ¹H-NMR, IC, and KF. Major peaks and their related intensities in the XRPD pattern are shown in Table 12. Benzenesulfonate Type A is an anhydrate.

The XRPD pattern of Compound 1 benzenesulfonate salt Type A is shown in FIG. 34. DSC and TGA results are shown in FIG. 35 and FIG. 36.

### Example 31: Preparation of Compound 1 naphthalene-1,5-disulfonate Salt Type A

Approximate 50 mg of Compound 1 freebase Type A and 1 equivalent of naphthalene-1,5-disulfonic acid were added in 1 mL methylethylketone. Obtained mixtures were stirred at 50 [symbol] for 2 hours and then at 25 [symbol] for 48 hours. Obtained suspension were filtered through a 0.45 μm nylon membrane filter by centrifugation at 14,000 rpm. After being dried at 50 [symbol] under vacuum for about 2 h, the solids were characterized by XRPD, DSC, TGA, ¹H-NMR, IC, and KF. Major peaks and their related intensities in the XRPD pattern are shown in Table 13. Naphthalene-1,5-disulfomate Type A is a hydrate.

The XRPD pattern of Compound 1 naphthalene-1,5-disulfonate salt Type A is shown in FIG. 37. DSC and TGA results are shown in FIG. 38 and FIG. 39.

### Example 32: Preparation of Compound 1 naphthalene-1,5-disulfonate Salt Type B

Approximate 50 mg of Compound 1 freebase Type A and 1 equivalent of naphthalene-1,5-disulfonic acid were added in 1 mL tetrahydrofuran. Obtained mixtures were stirred at 50 [symbol] for 2 hours and then at 25 [symbol] for 48 hours. Obtained suspension were filtered through a 0.45 μm nylon membrane filter by centrifugation at 14,000 rpm. After being dried at 50 [symbol] under vacuum for about 2 h, the solids were characterized by XRPD, DSC, TGA, ¹H-NMR, IC, and KF. Major XRPD peaks and their related intensities in the XRPD pattern are shown in Table 14. Naphthalene-1,5-disulfonate Type B is a hydrate.

The XRPD pattern of Compound 1 naphthalene-1,5-disulfonate salt Type B is shown in FIG. 40. DSC and TGA results are shown in FIG. 41 and FIG. 42.

### Example 33: Characterization of Salt Forms

| | HCI salt Type A, | Sulfate Type A, | Sulfate Type B, |
|---|---|---|---|
| | anhydrate | anhydrate | anhydrate |
| Preparation solvent | THF | THF | MEK |
| Melting onset (by | 180.1 | 140.5 | 164.3 |
| DSC, [symbol] ) | | | |
| Enthalpy (by DSC, | Decomposition occurred | Decomposition occurred | Decomposition occurred |
| J/g) | upon melting | upon melting | upon melting |
| Weight loss (by | 1.4% @160 [symbol] , 10.6% | 1.3% @ 135 [symbol] , 13.4% | 1.1% @ 150 [symbol] |
| TGA) | from 160 [symbol] to 206 [symbol] | from 135 [symbol] to 160 [symbol] | |
| Stoichiometric ratio | 1:1.16 | 1:1.25 | 1:1.52 |
| (by IC) | | | |
| Residual solvent | No residual solvent | No residual solvent | 0.7% MEK by weight |
| (by ¹H-NMR) | | | (0.05 equiv.) |

| | | | disulfonate Type | Naphthalene-1,5- |
|---|---|---|---|---|
| | Methanesulfonate | Benzenesulfonate | Naphthalene-1,5- | disulfonate Type |
| | Type A, anhydrate | Type A, anhydrate | A, hydrate | B, hydrate |
| Preparation | THF | MEK | MEK | THF |
| solvent | | | | |
| Melting onset | 155.0 | 156.9 | Dehydration onset: | Dehydration onset: |
| (by DSC, [symbol] ) | (Decomposition | (Decomposition | 19.6, melting onset: | 13.4, melting onset: |
| | occurred upon | occurred upon | 174.8 | 191.5 |
| | melting) | melting) | (Decomposition | (Decomposition |
| | | | occurred upon | occurred upon |
| | | | melting) | melting) |
| Enthalpy (by | 121, 12 | 124 | 61, 59, 3 | 13, 67 |
| DSC, J/g) | | | | |
| Weight loss (by | 0.8% @140 [symbol] , | 1.0% @ 145 [symbol] , | 3.0% @ 110 [symbol] , | 1.1% @ 100 [symbol] , |
| TGA) | 16.5% from 140 [symbol] | 14.3% from 145 [symbol] | 0.9% from 110 [symbol] | 1.3% from 100 [symbol] |
| | to 184 [symbol] | to 180 [symbol] | to 170 [symbol] , 10.3% | to 188 [symbol] , 7.7% |
| | | | from 170 ° C. to | from 188 [symbol] to |
| | | | 210 [symbol] | 220 [symbol] |
| Stoichiometric | 1:1.0 | 1:1.0 | 1:0.99 | 1:1.8 |
| ratio (by ¹H- | | | | |
| NMR) | | | | |
| Residual solvent | No residual solvent | 0.4% MEK by | 0.3% MEK by | No residual solvent |
| (by ¹H-NMR) | | weight (0.04 equiv.) | weight (0.03 equiv.) | |
| KF | N/A | N/A | 3.2% water by | 1.2% water by |
| | | | weight (1.42 equiv.) | weight (0.68 equiv. |

### Example 34: Competitive Equilibration Experiments

To determine the relative stability of anhydrates, competitive equilibration experiments were conducted in different solvent systems.

Type C and Type F were firstly used for competitive slurry experiments to determine the relative thermodynamic stability. About 5 mg of Type F and 5 mg of Type C, were added to 0.5 mL saturated solutions of Type C in selected solvents (the saturated solutions were prepared by Type C and the corresponding solvent). Obtained suspensions were stirred at 5 [symbol] , 25 [symbol] and 50 [symbol] for 1 week, respectively. Solid parts (wet cakes) were isolated by filtration and investigated by XRPD.

The pure Type D cannot be obtained and only a physical mixture of Type A and Type D could be prepared. Based on the previous study, Type C was more stable than Type F. So the mixture of Type A and Type D was used for competitive slurry experiments with Type C. About 5 mg of Type D+A, and 5 mg of Type C were added into 0.5 mL saturated solutions of Type C in selected solvents. Obtained suspensions were stirred at 25 [symbol] and 50 [symbol] for 3 days, respectively. Solid parts (wet cakes) were isolated by filtration and investigated by XRPD.

The results showed Type C is the most thermodynamically stable form of the freebase.

TABLE 17
Competitive equilibration experiments
Initial Type: Type C + Type F

| | XRPD | | |
|---|---|---|---|
| Solvents | 5 [symbol] | 25 [symbol] | 50 [symbol] |
| Ethanol | Type C | Type C | Type C |
| DCM | Type F + E | Type F | // |
| MTBE | Type C | Type C | Type C |
| MeOH | Type C | Type C | Type C |
| IPA | Type C | Type C | Type C |
| Ethyl acetate | Type C | Type C | Type C |

TABLE 18
Competitive equilibration experiments
Initial Type: Type C + Type D + A

| | XRPD | | |
|---|---|---|---|
| Solvents | 5 [symbol] | 25 [symbol] | 50 [symbol] |
| MTBE | // | Type C | Type C |
| IPAc | // | Type C | Type C |
| MeOH | // | Type C | Type C |

Dynamic Vapor Sorption (DVS)

| | |
|---|---|
| Instrument | ProUmind High-throughput DVS |
| Total gas flow | 4000 sccm |
| Oven temperature | 25 [symbol] |
| Solvent | Water |
| Method | Cycle: |
| | 40-95-0-95-40% RH |
| | Stage Step: 10% |
| | Equilibrium: 240 min (time) |

Karl Fischer

| | |
|---|---|
| Instrument | Mettler Toledo Coulometric KF Titrator C30 |
| Method | Coulometric |

Nuclear Magnetic Resonance (NMR)

| | |
|---|---|
| Instrument | Bruker Avance-AV 400M |
| Frequency | 400 MHZ |
| Probe | 5 mm PABBO BB-1H/D |
| Number of scan | 8 |
| Temperature | 297.6K |
| Relaxation delay | 1 second |

High Performance Liquid Chromatograph (HPLC)

| Instrument | Thermo scientific Vanquish | | |
|---|---|---|---|
| HPLC method | Wave length: 290 nm | | |
| | Column: Waters XSelect CHS C18 (3.5 μm, 4.6*150 mm, PDS-HPLC- | | |
| | 442) | | |
| | Detector: DAD | | |
| | Column temperature: 25 [symbol] | | |
| | Flow rate: 1 mL/min | | |
| | Mobile phase A: 0.05% TFA in water (v/v) | | |
| | Mobile phase B: 0.05% TFA in ACN (v/v) | | |
| | Diluent: THF/water (1/1, v/v) | | |
| | Injection volume: 5 μL (0.3 mg/mL) | | |
| | Gradient: | | |
| | Time (min) | Mobile Phase A (%) | Mobile Phase B (%) |
| | 0.00 | 95 | 5 |
| | 10.00 | 70 | 30 |
| | 20.00 | 5 | 95 |
| | 22.00 | 5 | 95 |
| | 22.10 | 95 | 5 |
| | 30.00 | 95 | 5 |

X-ray Powder Diffractometer (XRPD)

| | |
|---|---|
| Instrument | Bruker D8 Advance |

Method 1 (Figure 5/example 2, Figure 17/example 5, Figure 37/example 10)

| | |
|---|---|
| X-ray geometry | Reflection |
| Detector | LYNXEYE_XE_T(1D mode) |
| Open angle | Max |
| Radiation | Cu/K-Alphal (λ =1.5406Å) |
| X-ray generator power | 40 kV, 40 mA |
| Primary beam path slits | Twin_Primary motorized slit 10.0 mm by sample length; |
| | SollerMount axial soller 2.5 [symbol] |
| Secondary beam path slits | Detector OpticsMount soller slit 2.5 [symbol] , Twin_Secondary motorized |
| | slit 5.2 mm |
| Scan mode | Continuous scan |
| Scan type | Locked coupled |
| Step size | 0.02 [symbol] |
| Time per step | 0.3 second per step |
| Scan range | 2 [symbol] to 40 [symbol] |
| Sample rotation speed | 15 rpm |
| Sample holder | Monocrystalline silicon, flat surface |

Method 2 (used in the rest XRPD figures and examples)

| | |
|---|---|
| X-ray geometry | Reflection |
| Detector | LYNXEYE_XE_T(1D mode) |
| Open angle | Max |
| Radiation | Cu/K-Alpha1 (λ = 1.5406Å) |
| X-ray generator power | 40 kV, 40 mA |
| Primary beam path slits | Twin_Primary motorized slit 10.0 mm by sample length; |
| | SollerMount axial soller 2.5 [symbol] |
| Secondary beam path slits | Detector OpticsMount soller slit 2.5 [symbol] , Twin_Secondary motorized |
| | slit 5.2 mm |
| Scan mode | Continuous scan |
| Scan type | Locked coupled |
| Step size | 0.02 [symbol] |
| Time per step | 0.06 second per step |
| Scan range | 3 [symbol] to 40 [symbol] |
| Sample rotation speed | 15 rpm |
| Sample holder | Monocrystalline silicon, flat surface |

Differential Scanning Calorimetric (DSC)

| | |
|---|---|
| Instrument | TA Discovery 2500 |
| Sample pan | Tzero pan and Tzero hermetic lid with a pin hole of 0.7 mm in |
| | diameter |
| Temperature range | 30 to 250 [symbol] or before decomposition |
| Heating rate | 10 [symbol] /min |
| Nitrogen flow | 50 mL/min |
| Sample mass | About 1-2 mg |

Thermal Gravimetric Analysis (TGA)

| | |
|---|---|
| Instrument | Discovery Q5000 |
| Sample pan | Aluminum, open |
| Start temperature | Ambient condition (below 35 [symbol] ) |
| Final temperature | 300 [symbol] or abort next segment if weight < 80% (w/w) |
| | (The weight loss of the compound is no more than 20% (w/w)) |
| Heating rate | 10 [symbol] /min |
| Nitrogen flow | Balance 10 mL/min; sample chamber 25 mL/min |
| Sample mass | About 2-10 mg |

Karl Fischer (KF)

| | |
|---|---|
| Instrument | Mettler Toledo Coulometric KF Titrator C30 |
| Method | Coulometric |
| Sample mass | About 10-20 mg |

Polarized Light Microscope (PLM)

| | |
|---|---|
| Instrument | Leica |
| Method | Crossed polarizer, silicone oil added |

Nuclear Magnetic Resonance (NMR)

| | |
|---|---|
| Instrument | Bruker Avance-AV 400 M (for ¹H-NMR) |
| Frequency | 400 MHZ |
| Probe | 5 mm PABBO BB/19F-1H/D Z-GRD Z108618/0406 (for ¹H-NMR) |
| Number of scan | 8 |
| Temperature | 297.6 K |
| Relaxation delay | 1 second |

Ion Chromatography (IC)

| | |
|---|---|
| Instrument | Metrohm 940 professional IC |
| Sample center | 889 IC |
| Detector | Conductivity detector |
| Eluent (anion) | 3.2 mmol/L Na₂CO₃ + 1.0 mmol/L NaHCO₃ |
| Eluent (cation) | 2.5 mmol/L MSA |
| Suppressor solutions | 0.5% H₂SO₄ |
| Column: | Anion A SUPP 5-150 or Cation Column C4-150 |
| Column temperature: | 30 [symbol] |
| Flow rate: | 0.7 mL/min (anion) or 0.9 mL/min (cation) |
| Diluent: | Acetone |
| Injection volume: | 20 μL |

High Performance Liquid Chromatograph (HPLC)

| Instrument | Agilent 1260 infinityII Binary Pump | | |
|---|---|---|---|
| HPLC method | Wave length: 290 nm | | |
| | Column: Waters XSelect CHS C18, 2.5 μm, 4.6*150 mm, | | |
| | Detector: DAD | | |
| | Column temperature: 25 [symbol] | | |
| | Flow rate: 1 mL/min | | |
| | Mobile phase A: 0.05% TFA in water | | |
| | Mobile phase B: 0.05% TFA in ACN | | |
| | Diluent: THF/water (1:1, v/v) or acetone | | |
| | Injection volume: 5 μL | | |
| | Conc.: 0.3 mg/mL | | |
| | | Gradient: | |
| | Time (min) | Mobile Phase A (%) | Mobile Phase B (%) |
| | 0.00 | 95 | 5 |
| | 10.00 | 70 | 30 |
| | 20.00 | 5 | 95 |
| | 22.00 | 5 | 95 |
| | 22.10 | 95 | 5 |
| | 30.00 | 95 | 5 |

What is claimed is:

1. A solid state form of tert-butyl 4-(6-(((6-cyanopyridin-3-yl)methyl)carbamoyl)-5-hydroxy-1,7-naphthyridin-2-yl)piperazine-1-carboxylate: 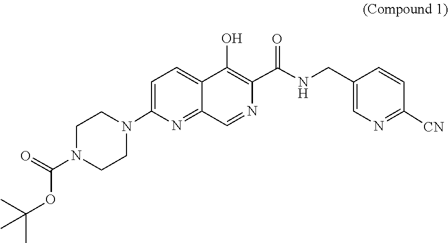 [SMILES: CC(C)(C)OC(=O)N1CCN(c2ccc3c(O)c(C(=O)NCc4ccc(C#N)nc4)ncc3n2)CC1]
or a pharmaceutically acceptable salt thereof.

2. The solid state form of claim 1, wherein the solid state form is a crystalline form.

3. The solid state form of claim 1 or 2, wherein the solid state form is crystalline Compound 1 as a freebase.

4. The solid state form of claim 1 or 2, wherein the solid state form is crystalline Compound 1 freebase Type C.

5. The solid state form of claim 1 or 2, wherein the solid state form is crystalline Compound 1 freebase Type A, crystalline Compound 1 freebase Type B, crystalline Compound 1 freebase Type C, crystalline Compound 1 freebase Type D, crystalline Compound 1 freebase Type E, crystalline Compound 1 freebase Type F, or crystalline Compound 1 freebase Type 1.

6. The crystalline form of claim 2, wherein the crystalline form is Compound 1 freebase characterized as having at least one of the following properties:
(a) an X-Ray powder diffraction (XRPD) pattern substantially the same as shown in FIG. 10 as measured using Cu Kα.radiation;
(b) an X-Ray powder diffraction (XRPD) pattern with peaks at 5.2±0.2° 2θ, 6.2±0.2° 2θ, and 14.7±0.2° 2θ as measured using Cu Kα.radiation;
(c) a Differential Scanning Calorimetry (DSC) thermogram substantially the same as shown in FIG. 11;
(d) a Differential Scanning Calorimetry (DSC) thermogram with an endothermic peak having a peak temperature at about 231.2 [symbol] ;
(e) a Thermogravimetric Thermal Analysis (TGA) thermogram substantially the same as shown in FIG. 12; or
(f) combinations thereof.

7. The crystalline form of claim 6, wherein the crystalline form has an X-ray powder diffraction (XRPD) pattern with peaks found in Table 4 as measured using Cu Kα.radiation.

8. The crystalline form of claim 6 or 7, wherein the crystalline form has an X-ray powder diffraction (XRPD) pattern with peaks at 5.2±0.2° 2θ, 6.2±0.2° 2θ, and 14.7±0.2° 2θ as measured using Cu Kα.radiation.

9. The crystalline form of any one of claims 6-8, wherein the X-ray powder diffraction (XRPD) pattern further comprises peaks at 13.7±0.2° 2θ, 17.9±0.2° 2θ, and 24.0±0.2° 2θ as measured using Cu Kα.radiation.

10. The crystalline form of any one of claims 6-9, wherein the X-ray powder diffraction (XRPD) pattern further comprises peaks at 11.5±0.2° 2θ and 21.2±0.2° 2θ as measured using Cu Kα.radiation.

11. The crystalline form of any one of claims 6-10, wherein the X-ray powder diffraction (XRPD) pattern further comprises peaks at 8.6±0.2° 2θ, 24.7±0.2° 2θ, and 28.3±0.2° 2θ as measured using Cu Kα.radiation.

12. The crystalline form of any one of claims 6-8, wherein the crystalline form has an X-ray powder diffraction (XRPD) pattern with peaks at 5.2±0.2° 2θ, 6.2±0.2° 2θ, 8.6±0.2° 2θ, 11.5±0.2° 2θ, 13.7±0.2° 2θ, 14.7±0.2° 2θ, 17.9±0.2° 2θ, 21.2±0.2° 2θ, 24.0±0.2° 2θ, 24.7±0.2° 2θ, and 28.3±0.2° 2θ as measured using Cu Kα.radiation.

13. The crystalline form of any one of claims 6-12, wherein the crystalline form is an anhydrate.

14. A pharmaceutical composition comprising a therapeutically effective amount of a crystalline form of any one of claims 2-13 and a pharmaceutically acceptable excipient.

15. A method of treating a disease or disorder in a subject, the method comprising administering to the subject a crystalline form of any one of claims 2-13 or a pharmaceutical composition of claim 14, wherein the disease or disorder is inflammatory bowel disease (IBD).

16. The method of claim 15, wherein the disease or disorder is ulcerative colitis (“UC”) or Crohn's disease (“CD”).
